(19) United States
(12) Patent Application Publication
(10) Pub. No.: US 2005/0112705 A1
(43) Pub. Date: May 26, 2005

(54) Variants of human kallikrein-2 and kallikrein-3 and uses thereof

(76) Inventors: Laurent Bracco, Paris (FR); Brigitta Brinkman, Clamart (FR); Fanny Coignard, Paris (FR)

Correspondence Address: NIXON & VANDERHYE, PC, 1100 N GLEBE ROAD, 8TH FLOOR, ARLINGTON, VA, 22201-4714, US

(21) Appl. No.: 10/503,990

(22) PCT Filed: Mar. 14, 2003
(86) PCT No.: PCT/FR03/00833

(30) Foreign Application Priority Data
Mar. 14, 2002 (FR) 02/03186
Sep. 5, 2002 (FR) 02/10975

(51) Int. Cl. G01N 33/574; C07H 21/04; C07K 14/575

(52) U.S. Cl. 435/7.23; 435/69.1; 435/320.1; 435/325; 530/350; 536/23.5

(57) ABSTRACT

The present invention pertains to the field of biology, genetics and medicine. It particularly pertains to new methods for detecting, characterising and/or treating cancers, particularly prostate cancer. The invention also pertains to methods for identifying or screening for compounds that exhibit activity in these diseases. The invention also relates to the compounds, genes, cells, plasmids or compositions that can be used to carry out the methods herein above. The invention particularly describes the role in these diseases of variants of human kallikrein 2 and human kallikrein 3, also known by the name PSA, and their use as therapeutic, diagnostic or experimental targets.

The present invention pertains to the field of biology, genetics and medicine. It particularly relates to new nucleotide sequences associated with alternative splicing events of genes corresponding to the PSA antigen (prostate specific antigen or KLK3) and to kallikrein-2 (KLK2). The invention also relates to methods for detecting the presence or for determining the level of expression of these nucleic acids or the corresponding proteins in biological samples, as well as to methods for selecting molecules capable of modulating their activity or their expression.

The invention is particularly adapted to the screening, prognosis, classification, or monitoring of cancers, in particular of prostate cancer, and in particular to differentiating between prostate cancer and benign hyperplasia (BPH), as well as to the development of new therapeutic approaches to these diseases.

Kallikreins correspond to a protein group the activity of which allows the post-translational modification of viral precursor proteins into biologically active forms. Certain members of this family, i.e. kallikrein 3, also known by the name PSA (“prostate-specific antigen”), and more recently kallikrein 2 are considered as the best markers available for detecting, diagnosing and monitoring prostate cancer. The use of tests measuring the PSA quantity in blood provides the possibility of making a diagnostic of a growing number of patients with prostate cancer (Pca). However, because PSA is also produced by non-cancerous, prostatic epithelial cells, it is often difficult to distinguish patients with prostate cancer from those with symptoms of benign prostatic hyperplasia (BPH). In the serum, PSA exists in a free, uncomplexed form, and in a complexed form, notably with alpha-antichymotrypsin. The measurement of these different forms and the ratio between them helps in the differential diagnosis of PCa and BPH.

Alternative splicing is a mechanism for regulating the expression of genes, which enables functional diversity to be generated from limited genetic information. This highly regulated mechanism can be subject to alterations during the development of human diseases. Thus, deregulation of the splicing machinery in cancer can lead to the expression of isoforms or variants that are specifically expressed in certain human tumours. These isoforms can have a decisive functional role in the development or maintenance of the disease's state. The specific expression of such isoforms constitutes a choice event for a rational and targeted approach to the development of medicinal products and/or diagnostic methods. A technology for profiling gene expression (DATAS) has recently been developed for identifying, in a systematic fashion, the genes and the domains within these genes that are susceptible to alteration by alternative splicing (WO99/46403).

The present invention now describes new genetic events associated with alternative splicing of PSA and KLK2 genes in prostatic tissues. The present invention is notably based on the construction of a repertoire of the splicing alterations associated with neoplastic prostate tissue, and the identification of structural alterations in the PSA and KLK2 genes, or in the corresponding mRNA. The present invention thus provides new therapeutic and diagnostic approaches of cancers, in particular of prostate cancer.

More particularly, a qualitative differential analysis was performed using RNA extracted from samples of prostatic tissues from tumour or non-tumoral areas of patients with carcinomas of the prostate. This analysis was performed using qualitative differential screening thanks to the implementation of the DATAS technique (described in the application no WO99/46403) which presents unequalled advantages. The application of DATAS technology to RNA molecules from neoplastic and non-neoplastic prostatic tissue has led to the isolation of various fragments of cDNA derived from the mRNA of human kallikrein 2 and kallikrein 3 (PSA). These results have then provided the possibility of identifying a certain number of cDNAs revealing events associated with alternative splicing.

The present invention therefore describes some original molecular events that can bring about the specific expression of isoforms or variants of KLK3 (PSA) and KLK2 in prostatic tissue and, more specifically, in cancerous tissue or tissue associated with benign prostatic hyperplasia (BPH). The present invention provides molecular data that justify the use of one or several of these variants as novel therapeutic and diagnostic targets, and which may be used to advantage in the diagnosis and treatment of cancers, and particularly prostate cancer.

A first aspect of the present invention relates to variants of human PSA and KLK2, in particular splicing variants. The invention relates to nucleic acids corresponding to these variants or to specific alterations that they present, as well as to the encoded proteins (or polypeptides or protein domains).

Another aspect of the present application relates to methods or tools for detecting the presence in biological samples (blood, plasma, urine, serum, saliva, biopsies or cell cultures, etc.) of these variants or alterations or for determining their respective quantity (quantities) or proportion(s). Such tools particularly comprise nucleic acid probes or primers, antibodies or other specific ligands, kits, devices, chips, etc. The detection methods can include hybridisation, PCR, chromatographic and immunological methods, etc. These methods are particularly adapted to detecting, characterising and monitoring disease progression or the efficacy of a treatment for cancers, in particular for prostate cancer or for determining predisposition to such a disease.

Another aspect of the present application relates to tools and methods for producing compounds active on the described variants, i.e. capable of modulating their expression or activity. These tools and methods particularly include nucleic acids, vectors, recombinant cells (or preparations derived from such cells), binding assays, etc. The invention is also intended to include compounds that are thus identified or produced, pharmaceutical compositions containing them, and their therapeutic uses.

The present invention is thus applicable to the diagnosis and to the development of therapeutic strategies of cancers, in particular of prostate cancer.

KLK2 and KLK3 Variants

A first aspect of the present application thus concerns KLK-2 and KLK-3 (PSA) variants or particular genetic alterations affecting these genes (or corresponding RNA or proteins). A more particular object of the invention relates to nucleic acids corresponding to these PSA and KLK2 variants or to specific alterations that they present, as well as to encoded proteins (or polypeptides or protein domains).

A certain number of isoforms of the KLK2 and KLK3 genes has been described in the prior art.

K-LM corresponds to the complete retention of intron 1 of KLK2 (Genbank accession number: AF336106) (David et al. (2002)). David et al. point out that the expression of K-LM messenger RNA is limited to prostatic epithelium and that the K-LM protein can be detected by immunohistochemistry in secretory epithelial cells (despite no data indicating the specificity of the antibody used). There are no data to indicate whether K-LM is present in human serum. K-LM seems to be detected in two samples of seminal fluid and tissue samples corresponding to benign prostatic hyperplasia. The endogenous form of K-LM could not be detected in prostate cell lines (with or without androgen stimulation). No results are shown on preferential or differential expression of K-LM in tissue or serum from patients with prostate cancer.

A KLK2 variant has been described that uses an alternative site between exon 4 and exon 5, corresponding to an open reading frame of 669 base pairs instead of 783 (Genbank accession number: S39329) (Riegman et al. (1991)).

Three variants with longer 3′UTR regions have been described (Liu et al. (1999)) (Genbank accession number: AF188745-7). One of these variants would have an open reading frame equivalent to wild-type KLK2; a second variant would have an open reading frame corresponding to that of the variant previously described (Riegman et al. (1991)). One of these variants has a 13-nucleotide deletion between exon 3 and exon 4, thus encoding a protein truncated by 97 amino acids in its carboxy-terminal part. The authors present some expression data using RT-PCR, but show no results on the corresponding protein or proteins.

PSA-LM corresponds to the complete retention of PSA intron 1 (David et al. (2002)) (Genbank accession number: AF335477, AF335478, AJ459784). David et al. point out that the expression of PSA-LM messenger RNA is limited to prostatic epithelium and that the PSA-LM protein can be detected by immunohistochemistry in secretory epithelial cells. There is no data to indicate the presence of PSA-LM in human serum, seminal fluid or tissues corresponding to benign prostatic hyperplasia. The endogenous form of PSA-LM could not be detected in prostate cell lines (with or without androgen stimulation). There are no results concerning the preferential or differential expression of PSA-LM in tissue or serum from patients with prostate cancer.

A PSA variant with a 129-nucleotide deletion in exon 3 has been described (Tanaka et al. (2000)). It is also known as PSA-RP3 (Heuzé-Vourc'h et al. (2003)). Tanaka et al. have shown qualitative expression data for this variant using RT-PCR in malignant and benign prostatic tissue. The expression of the corresponding protein has not been characterised.

Two PSA variants corresponding to complete retention of intron 3 (PA 424) and to partial retention of the last 442 nucleotides of intron 4 (PA 525) have been described (Genbank accession number: M21896, M21897) (Riegman et al. (1988)). PA 424 can give rise to a mature protein of 156 amino acids in length. The last 16 amino acids would be different from wild-type PSA. PA 525 would result in a mature protein of 214 amino acids. The last 28 amino acids would be different from wild-type PSA. Riegman et al. presented no additional data on the differential expression of messenger RNA or protein.

PA 424 and PA 525 described below are very similar to PSA-RP1 and PSA-RP2, which were isolated subsequently (Genbank accession numbers: AJ310937, AJ310938) (Heuzé et al. (1999); Heuzé-Vourc'h et al. (2001)). Although COS cell lines transfected with PSA-RP1 and PSA-RP2 cDNAs can express and secrete the corresponding proteins, Heuzé et al. showed no results demonstrating the expression of endogenous PSA-RP1 and PSA-RP2 proteins in prostate tissues.

Another group (Meng et al. (2002)) has characterised PSA-RP1 messenger RNA expression using Northern blots and in situ hybridisation. No difference in the expression could be observed between healthy and neoplastic microdissected tissue. It was possible to detect expression of the PSA-RP1 protein in the cytoplasm of epithelial cells by immunohistochemistry, using a specific PSA-RP1 antibody on sections of healthy and neoplastic prostate tissue.

A PSA variant corresponding to retention of the 5′ part of intron 4, PSA-RP5, has been submitted to Genbank (accession number: AJ512346)

A PSA variant with a deletion in exon 3, PSA-RP4, has been submitted to Genbank (accession number: AJ459782).

The present application now describes the existence of different forms of the PSA and KLK2 genes and their correlation with pathological situations. These isoforms have been identified from tumour samples. The description of cDNA and proteins/polypeptides encoded by these cDNA is indicated below. The full sequences are provided in the List of Sequences appended hereto. The main characteristics of the specific variants of the invention are described in the examples.

A first object of the invention relates to nucleic acids comprising the sequence of the PSA and KLK2 variants described in this application or a specific part thereof.

Another object of the invention relates to nucleic acids specific of the genetic alterations on the PSA and KLK2 variants described in this application. Such nucleic acids can particularly be complementary to mutated regions, retained intron domains or to junctions that have been newly created by deletions.

Another object of the invention relates to a nucleic acid comprising all or part of the sequence derived from messenger RNAs (or cDNAs) from KLK2-EHT002 to KLK2-EHT011 and from PSA-EHT001 to PSA-EHT027 or any combination of these variants as well as their uses to implement a method for diagnosing, detecting or monitoring cancers, in particular prostate cancer, and more particularly a benign form of the latter, BHP.

Another object of the invention lies in any nucleic acid wherein the nucleic acid comprises a sequence chosen among:
a) sequences SEQ ID NO: 1 to 49;
b) a variant of sequences SEQ ID NO: 1 to 49 resulting from the degeneracy of the genetic code;
c) the complementary strand of sequences SEQ ID NO: 1 to 49; and
d) a specific fragment of sequences a) to c).

The term “specific” fragment or part denotes a characteristic fragment of the concerned variants, typically a fragment containing at least a genetic alteration characteristic of the concerned variants. Such specific fragments differ therefore from the wild-type sequence by the presence of a particular structural feature (e.g. a mutation, a new junction, retention of an intron, deletion of a sequence, a stop codon, a new sequence resulting from a reading frame shift, etc.) resulting from an alteration event in patients demonstrated by the applicants. This particular structural feature is also denoted by the expression “target sequence”. Specific fragments according to the invention comprise at least a target sequence as defined above. Preferred fragments comprise at least 5 consecutive nucleotides of the concerned sequence, preferably at least 8, more preferably at least 12. The fragments may comprise up to 50, 75 or 100 nucleotides or more.

As used in the invention, nucleic acids can be DNA, preferably selected among cDNA and gDNA, or RNA. They can be synthetic or semi-synthetic nucleic acids, PCR fragments, oligonucleotides, double- or single-stranded regions, etc. The nucleic acids can be produced by synthesis, a recombinant pathway, cloning, gene assembly (or assemblies), mutagenesis, etc., or by using a combination of these techniques.

The nucleic acids can be used to produce a variant of PSA or KLK2 of the invention, either in vitro, ex vivo, in vivo, or in a cell-free transcription system. They can also be used in the production of antisense or interfering (RNAi) molecules capable of reducing the expression or translation of the corresponding mRNA in a cell. They can also be used to produce probes, particularly labelled probes, allowing through hybridisation reactions, the identification, in a specific manner, of the presence in a sample of a mutated form of PSA or KLK2 described in the invention. Furthermore, they can be used to produce nucleic acid primers that are useful for amplifying a variant of PSA or KLK2 (or a target sequence of such a variant) in a sample, particularly with the aim of screening for or diagnosing a disease.

In this regard, another object of the invention relates to a nucleic acid probe wherein the nucleic acid probe allows the detection of a nucleic acid as defined above, typically through selective hybridisation from a test nucleic acid population. In general, the probe comprises the sequence of a nucleic acid as defined above, or a (specific) part of the sequence of such a nucleic acid. The specific part is preferably characteristic of a variant as described herein above, and is particularly a part that contains an alteration associated with prostate cancer. It typically comprises from 10 to 1,000 nucleotides, preferably from 50 to 800, and is usually single-stranded. A particular example of a probe is represented by an oligonucleotide that is specific for and complementary to at least one region of a nucleic acid as defined herein above. The oligonucleotide is typically single-stranded and generally comprises from 10 to 100 bases. Specific examples of oligonucleotides covered by the invention are provided in Table 1. The oligonucleotides and/or nucleic acid probes of the invention may be labelled, for example by means of radioactive, enzymatic, fluorescent or luminescent markers, etc.

Another object of the invention relates to a nucleic acid probe allowing the (selective) amplification of a nucleic acid as defined herein above or of a (specific) part of such a nucleic acid. The amplified part preferably contains an alteration that is characteristic of any one of the variants described herein above, particularly an alteration associated with prostate cancer. A primer according to the invention is typically single-stranded, and is advantageously composed of 3 to 50 bases, preferably 3 to 40 and even more preferably 3 to 35 bases. A particular primer is complementary to at least one region of the PSA or KLK-2 gene or its corresponding RNA.

A preferred embodiment lies in a primer composed of a single-stranded nucleic acid comprising from 3 to 50 nucleotides complementary to at least a part of one of the sequences SEQ ID NO: 1 to 49 or their complementary strand. Examples of such nucleic acid primers can be found in the experimental section.

The invention also relates to a primer pair comprising a sense sequence and a reverse sequence, wherein the primers of said pair hybridise to a region of a nucleic acid as defined above and enable amplification of at least a portion thereof.

Particular primer pairs according to the invention are provided in Table 2.

Another object of the present application relates to any vector comprising a nucleic acid as defined above. It can be a plasmid, cosmid, episome, artificial chromosome, virus, phage, etc. Various commercially available plasmids can be mentioned, such as pUC, pcDNA, pBR, etc. Among the viral vectors, retroviruses, adenovirus, AAV, herpes virus, etc. can also be mentioned.

It is another object of the invention to provide recombinant cells containing a nucleic acid or a vector as defined herein above. The cells can be prokaryotic or eukaryotic. Among the prokaryotic cells, bacteria such as E. coli can be particularly mentioned. Among the eukaryotic cells, yeast cells or mammalian, insect or plant cells can be mentioned. They can be primary cultures or cell lines. COS, CHO, 3T3, HeLa, etc. cells can be mentioned.

Another object of the invention relates to a composition comprising a nucleic acid, as defined above, immobilised on a matrix (support). The invention particularly relates to compositions comprising a plurality of mixed nucleic acids in a soluble form or immobilised on a matrix, the composition comprising at least one nucleic acid as defined herein above.

Another object of the invention relates to a (product comprising a) matrix on which one or several nucleic acids as defined herein above are immobilised. The matrix can be solid, flat or otherwise, uniform or otherwise, such as for example nylon, glass, plastic, metal, fibre, a ceramic material, silica, a polymer, etc., or any other compatible material. The nucleic acids are preferably immobilised by one end, under conditions that render the molecule accessible for a hybridisation reaction. The nucleic acids can be arranged in a precise manner on the matrix, and deposited several times over.

In a particular variant, one or several specific oligonucleotide(s) is/are used to characterise each alternative splicing event (see FIG. 9). Notably, one could use an oligonucleotide specific for an eliminated exon, enabling quantification of the long form; and/or an oligonucleotide specific for one of the flanking exons that is not involved in splicing could be used to quantify long and short forms of the RNA; and/or one or several (e.g. three) oligonucleotides specific for the junctions could be used, one of which being specific for the new sequence generated after splicing, enabling quantification of the spliced form. Of course, other combinations of oligonucleotides can be envisaged, in particular the use of one or two oligonucleotides only. As far as junction oligonucleotides are more specifically concerned, they should ideally be centred on the junctions, although oligonucleotides that are shifted with respect to the junction can also be used. Advantageously, one would use oligonucleotides that have no secondary structure, which could interfere with their ability to hybridise. Generally, it is preferable for the chip if all the oligos generated have a uniform thermodynamic profile, namely in terms of Tm (65° C.) and length (24- or 25-mers). Furthermore, during their synthesis, the oligonucleotides can be modified by addition of a NH₂—C6 group to the 5′ end, promoting flexibility and enabling them to form a covalent bond with the polymer used to coat the matrix.

Another object of the invention relates to a (product comprising a) matrix on which one or several recombinant cells as defined herein above are immobilised or cultured. The matrix can be solid, flat or otherwise, uniform or otherwise, such as for example nylon, glass, plastic, metal, fibre, a ceramic material, silica, a polymer, etc., or any other compatible material. The cells are, for example, dispensed into the wells of a microtitre plate, or immobilised in a gel or on a suitable matrix.

The invention also pertains to the peptides and protein sequences encoded by all or part of the isoforms KLK2-EHT002 to KLK2-EHT011, and PSA-EHT001 to PSA-EHT027 or KLK2-EHTb to KLK2-EHTl or PSA-EHTa to PSA-EHTu particularly those described in sequences SEQ ID NO: 50 to 167 as well as their uses to implement a method for diagnosing, detecting or monitoring cancers, in particular prostate cancer, and more particularly a benign form of the latter, BHP.

A particular object of the present application relates to a polypeptide comprising all or a specific part of a sequence selected among SEQ ID NOs: 50 to 167. Particular polypeptides are composed of or comprise a sequence or part of a sequence created by the alteration of the gene or of the corresponding messenger. As used in the invention, the term “part” preferably denotes at least 5 contiguous residues, preferably at least 8, more preferably at least 10, still more preferably at least 15. As explained herein above, splicing alterations of the PSA or KLK2 gene lead to the production of mutated proteins that contain newly created sequences (target sequences). They can be new sequences (e.g. frame-shifted translation, insertions) or new junctions, etc. Particular peptides of the invention correspond to or include all or a specific part of sequences SEQ ID Nos: 53, 56, 59, 62, 65, 67 (residues 146-150), 70, 71, 73, 76, 79, 81, 93, 95, 98, 106, 108, 110, 112, 117, 119 (residues 66-70 or 74-79), 121 (residues 117-121), 123 (residues 25-29, 51-55 or 105-111), 126, 131, 133, 134, 135 (residues 64-68) and 155.

It is another object of the invention to provide a (product comprising a) matrix on which are immobilised one or several polypeptides as defined herein above. The matrix can be solid, flat or otherwise, uniform or otherwise, such as for example nylon, glass, plastic, metal, fibre, a ceramic material, silica, a polymer, etc., or any other compatible material. The polypeptides are preferably immobilised by one end, under conditions that leave the molecule accessible for a reaction involving interaction with a specific ligand, such as an antibody. The polypeptides can be arranged in a precise manner on the matrix, and deposited several times over.

Techniques for immobilising substances (such as nucleic acids, polypeptides, antibodies, etc.) on matrices have been described in the literature, and particularly in applications or patents nos. EP619 321, WO91/08307, U.S. Pat. No. 4,925,785 and GB2,197,720.

Specific Ligands

The invention also relates to specific ligands, preferably peptide ligands, particularly antibodies (polyclonal or monoclonal) and their fragments, which are specific for peptide regions characteristic of the proteins encoded by KLK2-EHT011 and PSA-EHT001-027 or by KLK2-EHTb to KLK2-EHTl and PSA-EHTa to PSA-EHTu (encoded by retained intron domains or specifically created junctions) and their uses for the detection, diagnosis or monitoring of cancers, in particular prostate cancer. In particular, it is suited to diagnosing the BPH form, and differentiating it from prostate cancer.

In this respect, another object of the invention relates to any antibody capable of binding, preferably in a selective manner, to a polypeptide as defined herein above. The antibody can be polyclonal or monoclonal. It can also be in the form of antibody fragments and derivatives with substantially the same antigenic specificity, in particular antibody fragments (e.g. Fab, F(ab′)2, CDRs), humanised, multifunctional, single chain (ScFv), etc. antibodies. The antibodies can be produced using conventional methods, comprising immunising an animal and collecting its serum (polyclonal) or spleen cells (in order to produce hybridomas by fusion with appropriate cell lines).

Methods for the production of polyclonal antibodies using various species have already been set out. Typically, the antigen is combined with an adjuvant (e.g. Freund's adjuvant) and administered to an animal, typically by subcutaneous injection. Repeated injections can be performed. Blood samples are collected and the immunoglobulin or serum is separated. Conventional methods for producing monoclonal antibodies comprise immunising of an animal with an antigen, followed by recovery of spleen cells, which are then fused with immortalised cells, such as myeloma cells. The resulting hybridomas produce monoclonal antibodies and can be selected by limiting dilution in order to isolate individual clones. Fab or F(ab′)2 fragments can be produced by digestion using a protease, according to conventional techniques.

The invention also relates to a method of producing antibodies, comprising injecting a polypeptide as defined herein above or an immunogenic fragment thereof into a non-human animal and recovering the antibodies or antibody-producing cells. The preferred antibodies are antibodies specific for the PSA and KLK2 isoforms described in the present application, and essentially non-specific for the wild-type forms.

The invention relates to hybridomas producing monoclonal antibodies described above and their use in producing said antibodies.

The antibodies can be coupled to heterologous fragments such as toxins, labels, medicinal products or any other therapeutic agent, in a covalent or non-covalent fashion, either directly, or through coupling agents. The labels can be chosen from among radio labels, enzymes, fluorescent agents, magnetic particles, etc.

The antibodies of the invention can be used as screening agents for detecting or quantifying the presence or quantity of PSA or KLK2 isoforms in samples taken from a subject, typically, a biological fluid taken from a mammal, for example a human.

It is another object of the invention to provide a (product comprising a) matrix on which are immobilised one or several antibodies (or fragments or derivatives) as defined herein above. The matrix can be solid, flat or otherwise, uniform or otherwise, such as for example nylon, glass, plastic, metal, fibre, a ceramic material, silica, a polymer, etc., or any other compatible material. The antibodies are preferably immobilised by one end, under conditions that leave the molecule accessible for a reaction involving interaction with a specific antigen. The antibodies can be arranged in a precise manner on the matrix, and deposited several times over.

Methods of Detection/Diagnosis

The present application describes new procedures for detecting in a subject a disease or predisposition to a disease, comprising determining the presence in a sample from said subject, of a nucleic acid, genetic alteration or a protein or a polypeptide as defined herein above.

The determination can be performed using different techniques, such as sequencing, selective hybridisation and/or amplification. Methods that can be used to determine the presence of proteins are based for example on immuno-enzymatic reactions, such as ELISA, RIA, EIA, etc. Techniques that can be used to determine the presence of altered genes or RNA are for example PCR, RT-PCR, the ligase chain reaction (LCR), the PCE technique or TMA (“Transcriptional Mediated Amplification”), gel migration, electrophoresis, particularly DGGE (“denaturing gel gradient electrophoresis”), etc.

In the case where an amplification step is performed, it is preferably achieved using a primer or a primer pair as defined herein above.

A particular object of the invention pertains to the use of nucleic acids that are complementary to and specific for fragments of the KLK2-EHT002-011 and PSA-EHT001-027 or KLK2-EHTb to KLK2-EHTl and PSA-EHTa to PSA-EHTu genes or messengers (e.g. retained intron domains, specifically created junctions, particular mutations, etc.) for detecting cancers, particularly prostate cancer, and more particularly its benign form, BPH. Cancer detection could in particular be achieved using DNA chips or by performing PCR on biological fluids such as blood (notably serum or purified circulating epithelial cells), urine or seminal fluid, etc.

The invention also resides in the development and use of immunological tests containing one or several antibodies as described herein above or fragments thereof. These assays can be used to detect and/or measure a variant individually, using a specific antibody, or several variants in parallel using suitable specific antibodies, or one or several ratios between the isoforms as described herein above or between said isoforms and other described forms of kallikrein 2 and PSA.

A particular method comprises contacting a sample taken from a subject with a nucleic acid probe as defined herein above, and demonstrating hybridisation.

Another particular method comprises contacting a sample taken from a subject with a primer or a primer pair as defined herein above, and demonstrating an amplification product.

Another particular method comprises contacting a sample taken from a subject with an antibody as defined herein above, and demonstrating an antigen-antibody complex.

Typically, several tests can be performed in parallel, using several samples and/or using several probes, primers and/or antibodies. Thus, in a particular embodiment, the procedure of the invention comprises determining the presence of several variants or genetic alterations in parallel, as described herein above, in a sample taken from a patient. The procedures of the invention can be carried out using a variety of biological samples, particularly biological fluids (e.g. blood, plasma, urine, serum, saliva, etc.), tissue biopsies or cell cultures, for example and, more generally, using any sample likely to contain nucleic acids or proteins (or polypeptides). The biological sample may be previously treated, in order to facilitate the procedure or to render the polypeptides or nucleic acids it contains more accessible. The sample can also be purified, centrifuged, fixed, etc., or possibly frozen or stored before use.

In a particular embodiment, the invention relates to a method for detecting the presence of an altered form of KLK2 or KLK3 in a subject, comprising contacting a sample from said subject, in vitro or ex vivo, with a probe, a primer or a specific ligand as defined herein above and determining respectively the formation of a hybrid, an amplification product or a complex, said formation being indicative of the presence of an altered form.

It is another object of the invention to provide a kit that can be used to carry out a method as defined herein above, comprising:
i) a pair of primers or a probe or an antibody as defined herein above, and
ii) the reagents required for an amplification or a hybridisation or an immunological reaction.

The invention also lies in the development of a method that allows to detect and/or measure the specific partners of one or several of these variants, by adding one or several of these variants or their fragments to biological fluid to be tested, such as blood (particularly serum), urine or seminal fluid.

Screening of Active Compounds

The specific variants of KLK2 and KLK3 of the invention were identified and isolated from diseased subjects and therefore represent particularly interesting therapeutic targets for treating cancers and particularly prostate cancer.

In this respect, it is a particular object of the invention to provide a method for selecting, identifying, characterising, optimising or producing active compounds, comprising a step determining the capacity of a test compound to modulate the expression or the activity of a polypeptide as defined herein above.

The compounds are more particularly selected on the basis of their capacity to modulate the synthesis of a polypeptide as defined herein above (i.e. particularly the production or maturation of the corresponding RNA molecules, or their translation) or the activity of such a polypeptide (i.e. particularly their maturation or transport, or their interaction with intra- or extracellular targets).

In a particular variant, the method comprises contacting a test compound in vitro or ex vivo with a polypeptide, as defined herein above, or a nucleic acid encoding such a polypeptide (e.g. a gene, cDNA, RNA), and selecting compounds that bind to said polypeptide or nucleic acid. Binding to the polypeptide, gene or corresponding RNA can be measured by various techniques, such as displacement of a labelled ligand, gel migration, electrophoresis, etc. It can be carried out in vitro, for example using the polypeptide or the nucleic acid immobilised on a matrix.

In another particular variant, the method comprises contacting in vitro or ex vivo a test compound with a cell expressing a polypeptide, as defined herein above, and selecting or identifying compounds that modulate the expression or the activity of said polypeptide. Modulation of the expression can be determined by assaying the RNA or proteins, or by means of an indicator system.

The cells used can be any compatible cell, particularly eukaryotic or prokaryotic cells as defined herein above. Typically, a cell is used that has been modified to express said molecule, particularly recombinant cells. Such recombinant cells can be prepared by the introduction of a recombinant nucleic acid that expresses the polypeptide, or a vector containing it. Such recombinant cells constitute particular objects of the invention.

The method can be carried out in order to select or identify activators or inhibitors of the expression or activity of the specific antigen of PSA or KLK2. The selection methods can be performed using various formats, such as, for example multi-well plates, in which multiple candidate compounds can be tested in parallel.

In a particular embodiment, the compound is an antisense nucleic acid capable of inhibiting the expression of the described variants. The antisense nucleic acid can comprise all or part of specific sequences of the described variants. The antisense sequence can notably comprise a region that is complementary to the identified splice form (e.g. a target sequence), and inhibit (or reduce) its translation into protein.

According to another embodiment, the compound is a chemical compound, of natural or synthetic origin, particularly an organic or inorganic molecule, of plant, bacterial, viral, animal, eukaryotic, synthetic or semi-synthetic origin, that is capable of modulating the expression or activity of one or several of the variants described herein above.

Specific compounds are preferred, i.e. those capable of modulating the expression or activity of the variants, without significantly affecting the expression or activity of wild-type forms.

The compound identified in this way can be used for preparing a composition for treating prostate cancer.

Another object of the invention resides in the use of a compound capable of modulating, i.e. stimulating, inhibiting or reducing the expression of one or several variants as described herein above, for preparing a composition intended for the treatment of cancer and particularly prostate cancer.

In the context of the invention, the term “treatment” denotes preventive, curative or palliative treatment, as well as patient management (reducing suffering, improving life expectancy, slowing the disease progression), etc. The treatment can moreover be carried out in combination with other active agents.

Another object of the invention relates to methods for selecting, identifying, or characterising active compounds that can be used for preparing compositions for treating cancerous conditions, comprising contacting one or several test compounds with cell extracts expressing the proteins described in the present invention, or with said proteins in a purified form.

The invention also relates to a method for producing a medicament for treating cancer, particularly prostate cancer, comprising (i) selecting active compounds according to the methods herein above and (ii) conditioning said compound or a functional analogue thereof in the presence of a pharmaceutically acceptable carrier. The functional analogue is typically a compound derived from the identified active compound, by chemical modification, particularly with the aim of improving its activity or pharmacokinetics, or with the aim of reducing its toxicity. The functional analogue can be a “prodrug” of the identified compound. Techniques for preparing functional analogues are well known to the skilled artisan, for example molecular modelling, coupling of NO groups, etc. The method can in this respect comprise an intermediate step of synthesising the selected compound or the functional analogue thereof.

The pharmaceutically acceptable carrier or excipient can be chosen from among buffer solutions, solvents, binders, stabilisers, emulsifiers, etc. Buffering solutions or diluents are particularly phosphate dicalcium, calcium sulphate, lactose, cellulose, kaolin, mannitol, sodium chloride, starch, powdered sugar and hydroxy propyl methyl cellulose (HPMC) (for slow release). Binders are for example starch, gelatine and filling solutions such as sucrose, glucose, dextrose, lactose, etc. Natural or synthetic gums can also be used, particularly alginate, carboxymethylcellulose, methylcellulose, polyvinyl pyrrolidone, etc. Other excipients are, for example, cellulose and magnesium stearate. Stabilising agents can be incorporated into the formulations, such as, for example polysaccharides (acacia, agar, alginic acid, guar gum and tragacanth, chitin or its derivatives and cellulose ethers). Solvents or solutions are for example Ringer's solution, water, distilled water, phosphate buffers, phosphate saline solutions, and other conventional fluids.

Another object of the invention pertains to the use of cytotoxic ligands specific for one or several variants as described herein above, which are localised on the surface of cancerous cells and, in particular, prostate cancerous cells.

Other aspects and advantages of the present invention will be apparent on reading the following examples, which should be considered as illustrative and non-limiting. These examples clearly indicate that the identified isoforms can be expressed in biological systems both at the RNA and protein level in tissues and serum.

### LEGENDS TO THE FIGURES AND TABLES

Table 1: Sequence of the specific oligonucleotides (SEQ ID NOs: 168-220). Column 1: Name of the oligonucleotide. Column 2: Oligonucleotide sequence. Column 3: SEQ ID NO of the claimed nucleotides.

Table 2: Primer pairs used for amplifying the PSA and KLK2 isoforms.

Table 3: Values of the fluorescence signals obtained by hybridisation of human tissues (Clontech) to an oligonucleotide microarray including oligonucleotide SEQ ID NOs: 168-220. Column 1: Name of the oligonucleotide. Column 2: SEQ ID NO. Column 3-4: Values corresponding to prostate/heart. Column 5-6: Values corresponding to prostate/kidney. Column 7-8: Values corresponding to prostate/prostate. Column 9-10: Values corresponding to prostate/small intestine. The sign #N/A indicates that the value was lower than twice the background noise.

Table 4: Values of the fluorescence signals obtained by hybridisation of cell lines to an oligonucleotide microarray including oligonucleotide SEQ ID NOs: 168-220. Column 1: Name of the oligonucleotide. Column 2: SEQ ID NO. Column 3-4: Values corresponding to Mda2b/BT549. Column 5-6: Values corresponding to Mda2b/MCF7. Column 7-8: Values corresponding to Mda2b/Mda231. Column 9-10: Values corresponding to Mda2b/T47D. The sign #N/A indicates that the value was lower than twice the background noise.

Table 5: Values of the fluorescence signals obtained by hybridisation of benign and neoplastic tissues from patients with prostate cancer to an oligonucleotide microarray including oligonucleotide SEQ ID NOs: 168-220. Column 1: Name of the oligonucleotide. Column 2: SEQ ID NO. Column 3-4: Values corresponding to neoplastic tissue/benign tissue from patient 15068. Column 5-6: Values corresponding to neoplastic tissue/benign tissue from patient 9648. Column 7-8: Values corresponding to neoplastic tissue/benign tissue from patient 8827. Column 9-10: Values corresponding to neoplastic tissue/benign tissue from patient 10063. The sign #N/A indicates that the value was lower than twice the background noise.

FIG. 1. Position of the specific oligonucleotides. The oligonucleotides marked by rectangles were designed to hybridise specifically to the splicing events: retention of an intron, deletion of an exon, use of 3′ and 5′ cryptic sites.

FIG. 2. Position of the specific oligonucleotides. Five oligonucleotides (marked by a line) can de designed to analyse the expression of a long form containing 3 exons and a short form containing 2 exons.

FIG. 3. Labelling of long and short synthetic forms. Synthetic RNAs are produced using linearised plasmids expressing the corresponding cDNAs. The RNAs from the long form are labelled with cyanine 3 and the RNAs from the short form are labelled with cyanine 5.

FIG. 4. Demonstration of the specificity of hybridisation of the oligonucleotides. Five oligonucleotides were used to distinguish long forms from short ones, mixed in equal quantities. Two examples are shown: gene A and gene B.

FIG. 5. Quantitative measurement of the ratio of long forms to short forms. The percentage of long forms (wt) was set at: 0, 20, 40, 60, 80 and 100 % (3 examples are shown, gene A, B and C).

FIG. 6. Specificity of the PSA and KLK2 oligonucleotide microarray. PSA-specific oligonucleotides are revealed by PSA isoforms labelled with cyanine 3. KLK2-specific oligonucleotides are shown by KLK2 isoforms labelled with cyanine 5.

FIG. 7. Diagram of linear RNA amplification.

FIG. 8. Example of hybridisation of the PSA/KLK2 slide using probes from neoplastic and healthy tissues from a single patient.

FIG. 9. Measuring the differential expression of certain isoforms of PSA and KLK2 by analysing neoplastic tissue and healthy tissues from the same patient with the corresponding discriminating oligonucleotides. Column 1: nature of the isoform, column 2: corresponding discriminating oligonucleotide, columns 3 to 6: log2 (ratio neoplastic expression/normal expression).

FIG. 10. Measuring the differential expression of certain isoforms of PSA and KLK2 by analysing prostate cancer cell lines (Mda-2b and LNCap) and a breast cancer cell line (T47D) with the corresponding discriminating oligonucleotides. Column 1: nature of the isoform, column 2: corresponding discriminating oligonucleotide, column 3, 4: log2 (ratio of prostate cell line expression/breast cell line expression). Isoforms that were relatively overexpressed in prostate cell lines are shown in orange. Isoforms that were relatively overexpressed in the breast cell line are shown in blue.

FIG. 11. Measuring the differential expression of certain isoforms of PSA and de KLK2 by analysing different human tissues with the corresponding discriminating oligonucleotides. Column 1: nature of the isoform, column 2: corresponding discriminating oligonucleotide, columns 3, 4, 5 and 6: log2 (ratio of prostate tissue expression/expression in heart, kidney, small intestine and prostate tissues, respectively). Isoforms that were relatively overexpressed in prostate tissue are shown in orange. The isoforms that were relatively overexpressed in other tissues are shown in blue.

FIG. 12. Graph showing the fluorescence signals obtained for certain isoforms with normal tissues.

FIG. 13. PCR amplification using specific oligonucleotide primers for three PSA isoforms. A) PSA-EHT003 B) PSA-EHT023 and C) PSA-EHT012

FIG. 14. Annotation of three polyclonal antibodies produced. This figure shows information on antibodies SE3962, SE3963 and SE4101, the chosen epitopes, the peptides synthesised, KLH conjugation and the isoforms likely to be recognised by these antibodies.

FIG. 15. Titres of the three antibodies using ELISA.

Determination of the titres of SE3962 in A), SE3963 in B) and SE4101 in C).

PPI: preimmune sera

PP: sera from the first harvest

GP: sera from the second harvest

FIG. 16. Results of western blots using the antibody EHT-SE3962 and sera containing a low concentration of total PSA in A), a moderate concentration of total PSA in B), a high concentration of total PSA in C). Two bands corresponding to the expected molecular weights of KLK2-EHT004 and KLK2-EHT006 are observed. The specificity of this antibody is demonstrated by the fact that the signals are displaced by increasing concentrations of the specific synthetic epitope (from 1 to 50 μg) but this is not observed with a high dose (250 μg) of non-specific peptide.

FIG. 17. Results of western blots using antibody EHT-SE3963 on prostate tissue. A band corresponding to the expected molecular weight for PSA-EHT021 is observed. Two other bands of greater molecular weight are also revealed.

### EXAMPLES

### A—Isolation of PSA and KLK2 Variants

Qualitative differential analysis was performed using polyadenylated (poly A+) RNA extracted from neoplastic and normal prostate samples. Poly A+ RNA is prepared using techniques known to those skilled in the art. In particular, it can involve treatment with chaotropic agents such as guanidinium thiocyanate followed by extraction of the total RNA by means of solvents (phenol or chloroform, for example). Such methods are well known to those skilled in the art (see Maniatis et al., Chomczynsli et al., Anal. Biochem. 162 (1987) 156), and can be carried out easily using commercially available kits. Poly A+ RNA is prepared from this total RNA according to conventional methods known to those skilled in the art and available in commercial kit form.

This poly A+ RNA is used as a template for reverse transcription reactions using reverse transcriptase. Advantageously, the reverse transcriptases used should have no RNase H activity. Longer strands of complementary DNA are obtained with these than with conventional reverse transcriptases. Such reverse transcriptase preparations with no RNase H activity are commercially available.

In accordance with the DATAS technique, hybridisations are performed for each time point of the kinetics between mRNA (C) and cDNA (T), as are reciprocal hybridisations between mRNA (T) and cDNA (C).

These mRNA/cDNA heteroduplexes are then purified according to DATAS technique protocols.

The RNA sequences that are not paired with complementary DNA are freed from these heteroduplexes by the action of RNase H, as this enzyme degrades unpaired RNA sequences. These unpaired sequences represent the qualitative differences that exist between RNA molecules that are otherwise homologous. These qualitative differences can be localised anywhere in the sequence of the RNA molecules, either 5′, 3′ or in the sequence and particularly in the coding sequence. Depending on their localisation, these sequences can not only be modifications due to splicing, but also the consequence of translocations or deletions.

The RNA sequences that represent qualitative differences are then cloned according to techniques known to those skilled in the art and particularly those described in the DATAS technique patent.

These sequences are grouped into cDNA libraries that constitute the qualitative differential libraries. One of these libraries contains the exons and introns specific to the healthy situation; the other libraries contain the splicing events that are characteristic of the pathological conditions.

The fragments derived from the human KLK2 and KLK3 genes come from these libraries.

Four neoplastic samples were mixed to form a tumour “pool”. This RNA pool was treated with DNase using a “DNA free” kit from the company Ambion (cat. no 1906).

This RNA molecule is then reverse transcribed using the reverse transcriptase supplied with the “High capacity cDNA Archive” kit, from the company Applied Biosystems (cat. no 4322171).

The cDNA thereby produced is used as a template for PCR reactions, in order to amplify specifically different regions of the messenger RNA molecules derived from human kallikrein 2 and kallikrein 3 according to the following protocol:

| | |
|---|---|
| Invitrogen 10× buffer: | 2 μl |
| DNTPs 2 mM: | 2 μL |
| MgCl2 50 mM: | 0.6 μL |
| Upstream primer 10 μM: | 0.4 μL |
| Downstream primer 10 μM: | 0.4 μL |
| Taq polymerase: | 0.2 μL |
| H2O: | 13.4 μL |
| cDNA | 1 μL |
| Final volume: | 19 μL |

Using the following PCR conditions:

| | | |
|---|---|---|
| 94° C. | 3 | min |
| 94° C. | 30 | sec ) |
| 55° C. | 1 | min ) 35 cycles |
| 72° C. | 3 | min ) |
| 72° C. | 6 | min |

The oligonucleotides used as PRC primers are the following:

For KLK2:

| | | |
|---|---|---|
| 163 | KLK2-1-S | GGTTCTCTCCATCGCCTTG |
| 164 | KLK2-1-AS | CTCCTTTAGTCTGAAGCCTCACC |
| 165 | KLK2-2-S | TGTATTTCACCACGACTATATCTCCC |
| 166 | KLK2-2-AS | GCTCTAGCACACATGTCATTGGA |
| 167 | KLK2-3-S | CAGTCATGGATGGGCACACT |
| 168 | KLK2-3-AS | CTCAGACCCAGGCATCTGG |
| 169 | KLK2-4-S | GCCAGATGGTGTAGCTGGG |
| 170 | KLK2-4-AS | CATGATGTGATACCTTGAAGCACC |
| 171 | KLK2-5-S | CCCTATCCAATTCTTTTGGGT |
| 172 | KLK2-5-AS | GCTTTGATGCTTCAGAAGGC |
| 173 | KLK2-6-S | CCTGCCAAGATCACAGATGTTG |
| 174 | KLK2-6-AS | TGGTTAGCTTTCAGATTGCAGC |
| 212 | KLK2-7-S | tgggaagaagaacaacgagca |
| 213 | KLK2-7-AS | tttagggaatcagagaactggcc |
| 214 | KLK2-8-S | agctcaatgtgtgtgcatgtgag |
| 215 | KLK2-8-AS | aaaggatgcgggaagtcaga |
| 216 | KLK2-9-S | cagcataattcacccattc |
| 217 | KLK2-9-AS | tctacctgttcactgctgcttcc |
| 218 | KLK2-10-S | ggagtgacgatgaggatgacc |
| 219 | KLK2-10-AS | gtcagttcagtgatcagaatgac |
| 220 | KLK2-11-AS | gctacagctgaaaccagcc |
| 221 | KLK2-12-S | ccactacagagccctcactcca |
| 222 | KLK2-13-AS | aatgcttctcacactcccagc |
| 249 | klk2 start | CCTGTGTCAGCATGTGGGACC |
| 250 | klk2 stop | TGGGACAGGGGCACTCAGGG |
| 251 | klk2e spe | cctgggggtatagttgccactat |

For PSA:

| | | |
|---|---|---|
| 175 | PSA-1-S | CGTGACGTGGATTGGTGAGA |
| 176 | PSA-1-AS | GCTGGCCTTAGAGGTTATCCTG |
| 177 | PSA-2-S | GGCCTGAACTGTGTCTTCCC |
| 178 | PSA-2-AS | GTGAACTTGCGCACACACG |
| 179 | PSA-3-S | TGGCAGGTGCTTGTGGC |
| 180 | PSA-3-AS | CTCCTCCCTCAGACCCAGG |
| 181 | PSA-4-S | GTCCAGCCCACAACAGTG |
| 182 | PSA-4-AS | CCTTGAAGCACACCATTACAGAC |
| 183 | PSA-5-S | CCTAAATCCATCTCCTATCCGAGTC |
| 184 | PSA-5-AS | CAGGATGAAACAGGCTGTGC |
| 185 | PSA-6-S | TGCTGTGAAGGTCATGGACC |
| 186 | PSA-6-AS | GACGCCTTGTTGGCTTCTAGAC |
| 200 | PSA-7-S | tcccagagaccttgatgctt |
| 201 | PSA-7-AS | gtttgcaggttggtggctg |
| 202 | PSA-8-S | gtcccggttgtcttcctcac |
| 203 | PSA-8-AS | gacccatttgttgtctcaggc |
| 204 | PSA-9-S | ctgaacacacgcacgggat |
| 205 | PSA-9-AS | ccaaagcccttccttttctca |
| 206 | PSA-10-S | ttggaaacccacgccaaa |
| 207 | PSA-10-AS | cctcagagtggctcagctgtag |
| 208 | PSA-11-S | tgactccctcaaggcaataggtta |
| 209 | PSA-11-AS | tgtttgctcactcccaccttct |
| 210 | PSA-12-S | tgctggacagaagcaggaca |
| 211 | PSA-12-AS | atcatcactccctccacatcc |
| 247 | PSA start | GGAGAGCTGTGTCACCATGTGG |
| 248 | PSA stop | ATAGGGGTGCTCAGGGGTTGG |

The amplified products are then cloned in the “Topo” system, from the company Invitrogen (cat. no K4600) in accordance with the protocol supplied. The ligation products are transformed into the “Top 10” competent cells. The colonies are identified on agar/LB medium, supplemented with ampicillin.

The cDNA molecules present in these colonies are amplified individually by PCR amplification, using primers Sp6 and T7, according to the following protocol:

| | |
|---|---|
| Primer T7 10 μM: | 2 μL |
| Primer Sp6 10 μM | 2 μL |
| MgCl2 50 mM: | 1.2 μL |
| DNTPs 2 mM: | 4 μL |
| 10× buffer | 4 μL |
| Taq polymerase: | 0.2 μL |
| H2O: | 25.6 μL |
| Colony: | 1 μL |
| final volume | 40 μL |

using the following PCR conditions:

| | | |
|---|---|---|
| 94° C. | 5 | min |
| 94° C. | 30 | sec |
| 55° C. | 30 | sec 30 cycles |
| 72° C. | 1 | mn |
| 72° C. | 5 | min |

The amplification products are then purified with P100 for sequencing, using the “Big Dye Terminator” kit from the company Applied Biosystems, according to the protocol provided by this supplier. The sequence reactions are analysed using a sequencer 3100 from Applied Biosystems. The table 2 shows the various cDNAs, as well as the oligonucleotide primer pairs used to obtain and amplify them in a sample.

### B—Identification and Description of the Variants

KLK2 Variants

The numbering of the nucleotides refers to GenBank accession number M18157, unless otherwise stated. The reference protein is the KLK2 equipped with its signal peptide.

Sequences KLK2-EHT002 to KLK2-EHT011 (SEQ ID NOs: 1 to 7) correspond to sequences with an open reading frame and an initiation and stop codon for translation.

Sequences KLK2-EHTb to KLK2-EHTl (SEQ ID NO: 8 to 15) correspond to expressed “EST” sequences, which can have one, two or three reading frame(s) with or without an initiation or stop codon for translation.

KLK2-EHT102 (SEQ ID NO: 1):

This isoform exhibits i) partial retention of a 5′ part of intron 2 (nt 1935-2020) and ii) use of two cryptic splice sites in the 3′ part of exon 3 (nt 3728) and the 5′ part of exon 4 (nt 3937). These two events correspond to consensus splice sites. The KLK2-EHT002 isoform has a stop codon after exon 2 and thus encodes a protein that is truncated after residue no. 69 (KLK2-EHT002prota/SEQ ID NO: 50). 54 amino acids can be cleaved to form sequence KLK2-EHT002protb/SEQ ID NO: 51. It can be seen that the nucleotides corresponding to Genbank (M18157) positions 1821 and 3581 in SEQ ID NO: 1 are C and A, whereas the Genbank reference sequence indicates T and G respectively at these positions. These differences can be explained by the existence of a polymorphism at these positions or by errors in the referenced sequence, although polymerase-induced mutations cannot be excluded. Neither change affects the sequence of the translated protein.

KLK2-EHT003 (SEQ ID NO: 2):

This isoform exhibits i) complete deletion of exon 2 and ii) retention of a 5′ part of intron 4 (nt 4061-4097). Both events correspond to consensus splice sites. The KLK2-EHT003 isoform codes for a protein with 34 additional amino acids beyond threonine residue number 15 (KLK2-EHT003prota/SEQ ID NO: 52). These 34 amino acids can be cleaved to form sequence KLK2-EHT003protb/SEQ ID NO: 53. It can be seen that the nucleotides corresponding to Genbank (M18157) positions 3774 and 5486 in SEQ ID NO: 2 are C and T, whereas the Genbank reference sequence indicates T and G respectively at these positions. These differences can be explained by the existence of a polymorphism at these positions or by errors in the referenced sequence, although polymerase-induced mutations cannot be excluded. Neither change affects the sequence of the translated protein.

KLK2-EHT004 (SEQ ID NO: 3):

This isoform has complete deletion of exon 3. The KLK2-EHT004 isoform encodes a protein with 70 additional amino acids beyond threonine residue number 15 (KLK2-EHT004prota/SEQ ID NO: 54). These 70 amino acids can be cleaved to form sequence KLK2-EHT003protb/SEQ ID NO: 55. The last 16 amino acids are new and could contain one or more of the specific epitopes of this isoform, KLK2-EHT004protc/SEQ ID NO: 56. It can be seen that the nucleotide corresponding to Genbank (M18157) position 4097 in SEQ ID NO: 3 is an A, whereas the Genbank reference sequence indicates a G at this position. This difference can be explained by the existence of a polymorphism at this position or by errors in the referenced sequence, although polymerase-induced mutations cannot be excluded. This change does not affect the sequence of the translated protein.

KLK2-EHT006 (SEQ ID NO: 4):

This isoform uses two cryptic splice sites in the 3′ part of exon 3 (nt 3728) and the 5′ part of exon 4 (nt 3937). This event corresponds to consensus splice sites. The KLK2-EHT006 isoform encodes a protein of 149 amino acids in length (KLK2-EHT006prota/SEQ ID NO: 57).134 amino acids can be cleaved to form the sequence KLK2-EHT002protb/SEQ ID NO: 58. The 16 last amino acids are new and could contain one or more of the specific epitopes of this isoform, KLK2-EHT004protc/SEQ ID NO: 59. It can be seen that the nucleotide corresponding to Genbank (M18157) position 3689 in SEQ ID NO: 4 is a T, whereas the Genbank reference sequence indicates a C at this position. This difference can be explained by the existence of a polymorphism at this position or by errors in the referenced sequence, although polymerase-induced mutations cannot be excluded. This change does not affect the sequence of the translated protein.

KLK2-EHT007 (SEQ ID NO: 5):

KLK2-EHT007 exhibits retention of the 5′ part of intron 4. The KLK2-EHT007 isoform encodes a protein of 224 amino acids in length (KLK2-EHT007prota/SEQ ID NO: 60). 209 amino acids can be cleaved to form the sequence KLK2-EHT007protb/SEQ ID NO: 61. The 14 last amino acids are new and can present one or more specific epitopes of this isoform, KLK2-EHT004protc/SEQ ID NO: 62.

KLK2-EHT009 (SEQ ID NO: 6):

KLK2-EHT009 exhibits i) deletion of a sequence in exon 3 (nt 3671-3793) and ii) the use of a cryptic splice site in the 5′ part of exon 4 (nt 3937) (a consensus splice site). The KLK2-EHT009 isoform encodes a protein of 123 amino acids (KLK2-EHT009prota/SEQ ID NO: 63). 108 amino acids can be cleaved to form the sequence KLK2-EHT009protb/SEQ ID NO: 64. The 5 last amino acids are new and may form part of one or more of the specific epitopes of this isoform, KLK2-EHT004protc/SEQ ID NO: 65.

KLK2-EHT01 1 (SEQ ID NO: 7):

This isoform uses a cryptic splice site in the 5′ part of exon 4 (nt 4041). This event corresponds to consensus splice sites. The KLK2-EHT011 isoform encodes a protein of 165 amino acids (KLK2-EHT011prota/SEQ ID NO: 66). 150 amino acids can be cleaved to form the sequence KLK2-EHT011protb/SEQ ID NO: 67. At the final amino acid position, a phenylalanine residue has been replaced by a tryptophan residue and may form part of a specific epitope of this isoform.

KLK2-EHTb (SEQ ID NO: 8):

This isoform exhibits retention of a 5′ part of intron 1, followed by a deletion between positions 701 and 1058, inclusive. The KLK2-EHTb isoform encodes a protein with 104 additional amino acids beyond threonine residue number 15 (KLK2-EHTb1, SEQ ID NO: 68). These 104 amino acids can be cleaved to form sequence KLK2-EHTb2, SEQ ID NO: 69. The last 59 amino acids (KLK2-EHTb3, SEQ ID NO: 70) represent a new sequence compared to an isoform already described, K-LM (David et al. (2002)). It can be seen that the nucleotides at positions 97, 214 and 249 of SEQ ID NO: 8 are G, C and T, whereas the Genbank reference sequence indicates C, T and C respectively. These differences can be explained by the existence of a polymorphism at these positions or by errors in the referenced sequence, although polymerase-induced mutations cannot be excluded. Mutations 97 and 214 do not affect the sequence of the translated protein. Mutation 249 converts a serine residue into a phenylalanine residue. It can also be seen that nucleotides 1192-1199, GAAGAACA in the Genbank reference are replaced by nucleotides 303-306, AAAC in SEQ ID NO: 8. The last fifteen amino acids of KLK2-EHTb1 thus replace an open sequence comprising the 17 amino acids that constitute KLK2-EHTb4, SEQ ID NO: 71.

KLK2-EHTc (SEQ ID NO: 9):

This isoform uses a cryptic site in intron 1 at position 1157. The KLK2-EHTc isoform encodes a protein with 6 additional amino acids beyond threonine residue number 15 (KLK2-EHTc1, SEQ ID NO: 72). These 6 amino acids can be cleaved to form sequence KLK2-EHTc2, SEQ ID NO: 73. It can be seen that nucleotides 1192-1199, GAAGAACA in the Genbank reference sequence are replaced by nucleotides 71-74, AAAC in SEQ ID NO: 9. This change occurs after a stop codon.

KLK2-EHTd (SEQ ID NO: 10):

This isoform exhibits retention of a 5′ part of intron 1, followed by a deletion between positions 657 and 1209, inclusive. The KLK2-EHTd isoform encodes a protein including at least 41 additional amino acids (KLK2-EHTd1, SEQ ID NO: 74). These 41 amino acids can be cleaved to form the sequence KLK2-EHTd2, SEQ ID NO: 75. The last 11 additional amino acids (KLK2-EHTd3, SEQ ID NO: 76) represent a new sequence with respect to an isoform that has already been described, K-LM (David et al. (2002)). The sequence predicted by continued translation of intron 1 produces a protein of 83 amino acids after cleavage: KLK2-EHTd4, SEQ ID NO: 77.

KLK2-EHTe (SEQ ID NO: 11):

KLK2-EHTe exhibits an unknown sequence of 140 nucleotides, comprising exon 2 truncated at its 3′ end and exon 3. The KLK2-EHTe isoform encodes a protein with 19 additional amino acids beyond the glycine residue that occupies position number 52 (KLK2-EHTe1, SEQ ID NO: 78). These 19 amino acids represent the sequence KLK2-EHTe2, SEQ ID NO: 79.

KLK2-EHTf (SEQ ID NO: 12):

This isoform uses two cryptic splice sites, the first in the 3′ part of exon 2 (position 1876) and the second in exon 4 (position 3349). The KLK2-EHTf isoform encodes a protein with 57 additional amino acids between the histidine residue at position 49 and asparagine at position 70 (KLK2-EHTf1, SEQ ID NO: 80). These 57 amino acids represent the sequence KLK2-EHTf2, SEQ ID NO: 81. It can be seen that the nucleotide at position 269 of SEQ ID NO: 12 is a C, whereas the Genbank reference sequence indicates a T at this position. This difference can be explained by the existence of a polymorphism at this position or by errors in the referenced sequence, although a polymerase-induced mutation cannot be excluded. Mutation 269 converts a phenylalanine residue into a leucine residue.

KLK2-EHTj (SEQ ID NO: 13):

This isoform has a deletion in intron 2, between positions 2473 and 3001. KLK2-EHTj encodes a protein with one of the two reading frames corresponding to KLK2-EHTj1 (SEQ ID NO: 82), or KLK2-EHTj2 (SEQ ID NO: 83).

KLK2-EHTk (SEQ ID NO: 14):

This isoform uses two cryptic splice sites, the first in intron 4 at position 5049 and the second in exon 5 at position 5469. KLK2-EHTk encodes a protein with one of the two reading frames corresponding to KLK2-EHTk1 (SEQ ID NO: 84), or KLK2-EHTk2 (SEQ ID NO: 85).

KLK2-EHTl (SEQ ID NO: 15):

This isoform uses a cryptic site in intron 2, which occupies position 2991. KLK2-EHTk encodes a protein with one of the two reading frames that corresponds to KLK2-EHTl1 (SEQ ID NO: 86) or KLK2-EHTl2 (SEQ ID NO: 88).

PSA (or KLK3) Variants

The numbering of the nucleotides refers to GenBank accession number M27274, unless otherwise stated. The reference protein is the PSA equipped with its signal peptide.

Sequences PSA-EHT001 to PSA-EHT027 (SEQ ID NOs: 16 to 34) correspond to sequences with an open reading frame and an initiation and stop codon for translation.

Sequences PSA-EHTa to PSA-EHTu (SEQ ID NOs: 35 to 49) correspond to expressed “EST” sequences, which may have one, two or three reading frames, with or without an initiation or stop codon for translation.

PSA-EHT001 (SEQ ID NO: 16

This isoform exhibits retention of a deleted fragment of intron 1 (nt 721-811, then 971-1272). The PSA-EHT001 isoform encodes a protein of 51 amino acids (PSA-EHT001prota/SEQ ID NO: 89). 36 amino acids can be cleaved to form the sequence PSA-EHT001protb/SEQ ID NO: 90. It can be seen that the nucleotide corresponding to Genbank (M27274) position 738 in SEQ ID NO: 16 is a G whereas the Genbank reference sequence indicates T. This difference can be explained by the existence of a polymorphism at this position or by errors in the referenced sequence, although polymerase-induced mutations cannot be excluded. This change replaces a tryptophan residue with a glycine residue.

PSA-EHT003 (SEQ ID NO: 17):

This isoform exhibits retention of a deleted fragment of intron 1 (nt 721-874, then 920-1272). The PSA-EHT003 isoform encodes a protein of 89 amino acids (PSA-EHT003prota/ SEQ ID NO: 91). 74 amino acids can be cleaved to form the sequence PSA-EHT003protb/SEQ ID NO: 92. The 20 last acids (PSA-EHT003protc/SEQ ID NO: 93) represent new information compared to an isoform already described that has complete retention of intron 1.

PSA-EHT004 (SEQ ID NO: 18):

This isoform uses a 3′ cryptic splice site in intron 1 at position 1142 (consensus site). The PSA-EHT004 isoform encodes a protein of 47 amino acids (PSA-EHT004prota/SEQ ID NO: 94). 32 amino acids can be cleaved to form the sequence PSA-EHT004protb/SEQ ID NO: 95.

PSA-EHT005 (SEQ ID NO: 19):

This isoform exhibits retention of a deleted fragment in intron 1 (nt 721-792, then 1149-1272). The PSA-EHT005 isoform encodes a protein of 68 amino acids (PSA-EHT005prota/SEQ ID NO: 96). 53 amino acids can be cleaved to form the sequence PSA-EHT005protb/SEQ ID NO: 97. The last 28 acids (PSA-EHT005protc/SEQ ID NO: 98) represent new information compared to an isoform already described that has complete retention of intron 1.

PSA-EHT007 (SEQ ID NO: 20):

This isoform uses a 5′ cryptic splice site located in exon 1 at position 693 and a 3′ cryptic site located in intron 1 at position 1149. This PSA-EHT007 isoform encodes a protein of 23 amino acids (PSA-EHT007prota/SEQ ID NO: 99).

PSA-EHT008 (SEQ ID NO: 21):

This isoform uses a 3′ cryptic splice site in intron 1 at position 1202 (consensus site). This PSA-EHT008 isoform encodes a protein of 27 amino acids (PSA-EHT008prota/SEQ ID NO: 100). 12 amino acids can be cleaved to form the sequence PSA-EHT008protb/SEQ ID NO: 101. It can be seen that the nucleotide corresponding to Genbank (M27274) position 679 in SEQ ID NO: 21 is T, whereas the Genbank reference sequence indicates a G. This difference can be explained by the existence of a polymorphism at this position or by errors in the referenced sequence, although polymerase-induced mutations cannot be excluded. This change replaces a tryptophan residue with a leucine residue.

PSA-EHT009 (SEQ ID NO: 22):

This isoform exhibits retention of a deleted fragment of intron 2 (nt 2119-2447, then 2988-3226). This PSA-EHT009 isoform encodes a protein of 69 amino acids (PSA-EHT009prota/SEQ ID NO: 102). 54 amino acids can be cleaved to form the sequence PSA-EHT009protb/SEQ ID NO: 103. It can be seen that the nucleotide corresponding to Genbank (M27274) position 1966 in SEQ ID NO: 22 is A, whereas the Genbank reference sequence indicates a G. This difference can be explained by the existence of a polymorphism at this position or by errors in the referenced sequence, although polymerase-induced mutations cannot be excluded. This change does not affect the sequence of the protein. Other point mutations are identified after the stop codon.

PSA-EHT012 (SEQ ID NO: 23):

This isoform uses a 3′ cryptic splice site in intron 2 at position 2426 (consensus site). This PSA-EHT012 isoform encodes a protein of 83 amino acids (PSA-EHT012prota/SEQ ID NO: 104). 68 amino acids can be cleaved to form the sequence PSA-EHT004protb/SEQ ID NO: 105. The 14 last amino acids (PSA-EHT012protc/SEQ ID NO: 106) represent new information compared to wild-type PSA and are thus likely to include one or more of the specific epitopes of this isoform. These differences can be explained by the existence of a polymorphism at these positions or by errors in the referenced sequence, although polymerase-induced mutations cannot be excluded. Neither change affects the sequence of the translated protein

PSA-EHT013 (SEQ ID NO: 24):

This isoform uses a 3′ cryptic splice site in intron 1 at position 1945 (consensus site). This PSA-EHT013 isoform encodes a protein of 75 amino acids (PSA-EHT013prota/SEQ ID NO: 107). 60 amino acids can be cleaved to form the sequence PSA-EHT013protb/SEQ ID NO: 108. These 60 amino acids represent new information compared to wild-type PSA and are thus likely to include one or more of the specific epitopes of this isoform.

PSA-EHT015 (SEQ ID NO: 25):

This isoform uses a 5′ cryptic splice site located in exon 1 at position 703 and a 3′ cryptic site located in exon 2 at position 2030. The PSA-EHT015 isoform encodes a protein of 41 amino acids (PSA-EHT015prota/SEQ ID NO: 109). The 30 last amino acids (PSA-EHT015protb/SEQ ID NO: 110) represent new information compared to wild-type PSA and are thus likely to include one or more of the specific epitopes of this isoform. It can be seen that the nucleotide corresponding to Genbank (M27274) position 2094 in SEQ ID NO: 25 is C, whereas the Genbank reference sequence indicates T. This difference can be explained by the existence of a polymorphism at this position or by errors in the referenced sequence, although polymerase-induced mutations cannot be excluded. This change replaces a serine residue by a proline residue.

PSA-EHT016 (SEQ ID NO: 26):

This isoform uses a 3′ cryptic splice site in exon 2 at position 2053 (consensus site). This PSA-EHT016 isoform encodes a protein of 39 amino acids (PSA-EHT016prota/SEQ ID NO: 111). 24 amino acids can be cleaved to form the sequence PSA-EHT016protb/SEQ ID NO: 112. These 24 amino acids represent new information compared to wild-type PSA and are thus likely to include one or more of the specific epitopes of this isoform.

PSA-EHT018 (SEQ ID NO: 27):

This isoform exhibits retention of a deleted fragment of intron 2 (nt 2119-2588, then 3114-3226). This PSA-EHT018 isoform encodes a protein of 69 amino acids (PSA-EHT018prota/SEQ ID NO: 113). 54 amino acids can be cleaved to form the sequence PSA-EHT018protb/SEQ ID NO: 114. It can be seen that the nucleotide corresponding to Genbank (M27274) position 2545 in SEQ ID NO: 27 is a T, whereas the Genbank reference sequence indicates A. This difference can be explained by the existence of a polymorphism at this position or by errors in the referenced sequence, although polymerase-induced mutations cannot be excluded. This change does not affect the sequence of the protein.

PSA-EHT019 (SEQ ID NO: 28):

This isoform has deletion of a fragment located in exon 3 (nucleotide 3828-3933). This PSA-EHT019 isoform encodes a protein of 100 amino acids (PSA-EHT019prota/SEQ ID NO: 115). 85 amino acids can be cleaved to form the sequence PSA-EHT019protb/SEQ ID NO: 116. The 6 last amino acids (PSA-EHT019protc/SEQ ID NO: 117) represent new information compared to wild-type PSA and are thus likely to include one or more of the specific epitopes of this isoform. It can be seen that the nucleotides corresponding to Genbank (M27274) positions 3786 and 3943 in SEQ ID NO: 28 are T and A, whereas the Genbank reference sequence indicates C and C respectively. These differences can be explained by the existence of polymorphisms at these positions or by errors in the referenced sequence, although polymerase-induced mutations cannot be excluded. The first change does not affect the sequence of the protein. The second replaces a serine residue with an arginine residue.

PSA-EHT021 (SEQ ID NO: 29):

This isoform uses a 3′ cryptic splice site located in exon 3 at position 3885 (consensus site) and also has a deletion in the 3′ part of exon 3 (nucleotide 3903-4025). The PSA-EHT021 isoform encodes a protein of 177 amino acids (PSA-EHT021prota/SEQ ID NO: 118). 162 amino acids can be cleaved to form the sequence PSA-EHT021 protb/SEQ ID NO: 119. The new junctions created around residues 69 and 76 represent new information compared to wild-type PSA and are thus likely to include one or more of the specific epitopes of this isoform. It can be seen that the nucleotide corresponding to Genbank (M27274) position 1966 in SEQ ID NO: 29 is an A, whereas the Genbank reference sequence indicates a G. This difference can be explained by the existence of a polymorphism at this position or by errors in the referenced sequence, although polymerase-induced mutations cannot be excluded. This change does not affect the sequence of the protein.

PSA-EHT022 (SEQ ID NO: 30):

This isoform presents a deletion in the 3′ part of exon 3 (nucleotide 3903-4025). This PSA-EHT022 isoform encodes a protein of 220 amino acids (PSA-EHT022prota/SEQ ID NO: 120). 205 amino acids can be cleaved to form the sequence PSA-EHT022protb/SEQ ID NO: 121. The new junction created around residue 119 represents new information compared to wild-type PSA and is thus likely to include one or more of the specific epitopes of this isoform. It can be seen that the nucleotide corresponding to Genbank (M27274) position 1966 in SEQ ID NO: 30 is A, whereas the Genbank reference sequence indicates a G. This difference can be explained by the existence of a polymorphism at this position or by errors in the referenced sequence, although polymerase-induced mutations cannot be excluded. This change does not affect the sequence of the protein.

PSA-EHT022 (SEQ ID NO: 30) corresponds to a PSA variant submitted to Genbank on 24^{th} October 2002 (accession number: AJ459782).

PSA-EHT023 (SEQ ID NO: 31):

This isoform has a deletion of a fragment of exon 2 (nucleotides 1990-2040), the use of a 3′ cryptic site in exon 3 at position 3885 (consensus site) and retention of a 5′ fragment from intron 3 (nucleotides 4043-4060) (consensus site). This isoform encodes a protein of 207 amino acids (PSA-EHT023prota/SEQ ID NO: 122).192 amino acids can be cleaved to form the sequence PSA-EHT023protb/SEQ ID NO: 123. The new junctions created around residues 27 and 53 and in region 105-111 represent new information compared to wild-type PSA and are thus likely to include one or more of the specific epitopes of this isoform. It can be seen that the nucleotides corresponding to Genbank (M27274) positions 2060 and 5731 in SEQ ID NO: 31 are G and G, whereas the Genbank reference sequence indicates T and T, respectively. These differences can be explained by the existence of polymorphisms at these positions or by errors in the referenced sequence, although polymerase-induced mutations cannot be excluded. The first change replaces a cysteine residue with a glycine residue. The second does not affect the sequence of the protein.

PSA-EHT025 (SEQ ID NO: 32):

This isoform is deleted for exon 3. This isoform encodes a protein of 85 amino acids (PSA-EHT025prota/SEQ ID NO: 124). 70 amino acids can be cleaved to form the sequence PSA-EHT025protb/SEQ ID NO: 125. The last 16 amino acids (PSA-EHT025protc/SEQ ID NO: 126) represent new information compared to wild-type PSA and are thus likely to include one or more of the specific epitopes of this isoform. It can be seen that the nucleotides corresponding to Genbank (M27274) positions 2118-4186 and 5791 in SEQ ID NO: 32 are G and G, whereas the Genbank reference sequence indicates AT and C respectively. These differences can be explained by the existence of polymorphisms at these positions or by errors in the referenced sequence, although polymerase-induced mutations cannot be excluded. The concordance of the 3′ site in exon 2 and the 5′ site in exon 4 suggests a mutation introduced by polymerase in this region. The last change does not affect the sequence of the protein.

PSA-EHT026 (SEQ ID NO: 33):

This isoform has a deletion of a fragment located in exon 3 (nucleotide 3781-4025). This PSA-EHT026 isoform encodes a protein of 78 amino acids (PSA-EHT026prota/SEQ ID NO: 127). 63 amino acids can be cleaved to form the sequence PSA-EHT026protb/SEQ ID NO: 128.

PSA-EHT027 (SEQ ID NO: 34)

This isoform uses a cryptic splice site located at the 5′ end of exon 3 at position 3780 and is deleted for exon 4. This PSA-EHT027 isoform encodes a protein of 144 amino acids (PSA-EHT027prota/SEQ ID NO: 129). 129 amino acids can be cleaved to form the sequence PSA-EHT027protb/SEQ ID NO: 130. The 67 last amino acids (PSA-EHT027protc/SEQ ID NO: 131) represent new information compared to wild-type PSA and are thus likely to include one or more of the specific epitopes of this isoform. It can be seen that the nucleotide corresponding to Genbank (M27274) position 1966 in SEQ ID NO: 34 is A, whereas the Genbank reference sequence indicates a G. This difference can be explained by the existence of a polymorphism at this position or by errors in the referenced sequence, although polymerase-induced mutations cannot be excluded. This change does not affect the sequence of the protein.

PSA-EHTa (SEQ ID NO: 35):

This isoform presents a deletion of 91 nucleotides in the 5′ part of intron 1, followed by a deletion of the next 152 nucleotides (then returning to intron 1). The PSA-EHTa isoform encodes a protein of 90 amino acids (PSA-EHTa1, SEQ ID NO: 132), the last 75 amino acids of which can be cleaved (PSA-EHTa2, SEQ ID NO: 133). It represents different information from PSA and the last 44 amino acids (PSA-EHTa3, SEQ ID NO: 134) represent new information compared to a complete retention of intron 1 that has already been described (David et al. (2002)). Q replaces P at position 26 of the 74 last amino acids. It can be seen that the nucleotides at position 90 and 234 of SEQ ID NO: 35 are A and C, whereas the Genbank reference sequence indicates C and T. The G and C nucleotides at position 243 and 293 also differ from the Genbank reference. However, these two nucleotides actually correspond to a published genomic sequence (Genbank accession number: NT_{—}011190). These differences can be explained by the existence of a polymorphism at these positions or by errors in the referenced sequence, although polymerase-induced mutations cannot be excluded. Thus, a glutamine residue has replaced a proline residue (mutation 90), and a threonine residue has replaced an isoleucine residue (mutation 234).

PSA-EHTd (SEQ ID NO: 36):

This isoform has a deletion of the last 9 nucleotides of exon 2 and the first 243 nucleotides of exon 3. This PSA-EHTd isoform encodes a protein with an 84 amino acid deletion (PSA-EHTd1/SEQ ID NO: 135). A new domain is formed between cysteine residue 66 and threonine residue 151.

PSA-EHTf (SEQ ID NO: 37):

This isoform exhibits retention of the deleted intron 3, of a length of 105 nucleotides (2420-2526). The PSA-EHTf isoform encodes a protein that is truncated after asparagine residue number 69, which is itself substituted by a lysine residue (PSA-EHTf1, SEQ ID NO: 136). It can be seen that the nucleotide at position 56 of SEQ ID NO: 37 is G, whereas the Genbank reference sequence indicates A. This difference can be explained by the existence of a polymorphism at this position or by errors in the referenced sequence, although a polymerase-induced mutation cannot be excluded. This mutation replaces a histidine residue with an arginine residue.

PSA-EHTh (SEQ ID NO: 38):

This isoform results from the use of a cryptic splice site within intron 4 (at position 5472). This PSA-EHTh isoform encodes a protein with one of the two reading frames corresponding to PSA-EHTh1 (SEQ ID NO: 137), or PSA-EHTh2 (SEQ ID NO: 138). It can be seen that the nucleotides at position 79, 199 and 258 of SEQ ID NO: 38 are C, C and G, whereas the Genbank reference sequence indicates T, T and A. These differences can be explained by the existence of a polymorphism at these positions or by errors in the referenced sequence, although a polymerase-induced mutation cannot be excluded.

PSA-EHTj (SEQ ID NO: 39):

This isoform results from the use of a cryptic splice site within intron 4 (at position 5257). This PSA-EHTj isoform encodes a protein with one of the three reading frames corresponding to PSA-EHTj1 (SEQ ID NO: 139), or PSA-EHTj2 (SEQ ID NO: 140) or PSA-EHTj3 (SEQ ID NO: 141).

PSA-EHTk (SEQ ID NO: 40):

This isoform exhibits retention of a 3′ part of intron 3, then retention of a truncated intron 4 (between positions 4337 and 5516). This isoform encodes a protein with one of the three reading frames corresponding to PSA-EHTk1 (SEQ ID NO: 142), PSA-EHTk2 (SEQ ID NO: 144) or PSA-EHTk3 (SEQ ID NO: 144).

PSA-EHTl (SEQ ID NO: 41):

This isoform uses a cryptic site in exon 4 at position 4274 and another cryptic site in intron 4 at position 4538. It can be seen that the nucleotide at position 79 of SEQ ID NO: 41 is C, whereas the Genbank reference sequence indicates a T. This difference can be explained by the existence of a polymorphism at this position or by errors in the referenced sequence, although a polymerase-induced mutation cannot be excluded. PSA-EHTl encodes a protein with one of the three reading frames corresponding to PSA-EHTl1 (SEQ ID NO: 145), PSA-EHTl2 (SEQ ID NO: 146) or PSA-EHTl3 (SEQ ID NO: 147). In PSA-EHTl3, this mutation replaces an isoleucine residue with a threonine residue.

PSA-EHTm (SEQ ID NO: 42):

This isoform exhibits retention of a truncated intron 1 (between 1214 and 1755). PSA-EHTm encodes a protein with one of the three reading frames corresponding to PSA-EHTm1 (SEQ ID NO: 148), PSA-EHTm2 (SEQ ID NO: 149) or PSA-EHTm3 (SEQ ID NO: 150).

PSA-EHTn (SEQ ID NO: 43):

This isoform exhibits retention of a truncated intron 1 (between 1366 and 1736). PSA-EHTm encodes a protein with one of the three reading frames corresponding to PSA-EHTn1 (SEQ ID NO: 151), PSA-EHTn2 (SEQ ID NO: 152) or PSA-EHTn3 (SEQ ID NO: 153).

PSA-EHTp (SEQ ID NO: 44):

This isoform results from the use of a cryptic splice site in intron 1 (at position 1240). PSA-EHTp can encode a protein with 27 additional amino acids beyond the isoleucine residue at position 15 (PSA-EHTp1, SEQ ID NO: 154). These 27 amino acids, representing the sequence PSA-EHTp2 (SEQ ID NO: 155), can be released after cleaving.

PSA-EHTq (SEQ ID NO: 45):

This isoform exhibits retention of a truncated intron 2 (between positions 2740 and 3167). KLK2-EHTk encodes a protein comprising one of the two reading frames corresponding to KLK2-EHTq1 (SEQ ID NO: 156), or KLK2-EHTq2 (SEQ ID NO: 157).

PSA-EHTr (SEQ ID NO: 46):

This isoform exhibits retention of a truncated intron 2 (between positions 2589 and 3199). PSA-EHTm encodes a protein comprising one of the three reading frames corresponding to PSA-EHTr1 (SEQ ID NO: 158), PSA-EHTr2 (SEQ ID NO: 159) or PSA-EHTr3 (SEQ ID NO: 160).

PSA-EHTs (SEQ ID NO: 47):

This isoform exhibits retention of a truncated intron 4 (between positions 4516 and 4889). It can be seen that the nucleotides at position 54, 93 and 201-208 of SEQ ID NO: 47 are C, A and TGCCGCTG, whereas the Genbank reference sequence indicates T, G and AG-GTGT. These differences can be explained by the existence of a polymorphism at these positions or by errors in the referenced sequence, although a polymerase-induced mutation cannot be excluded. This isoform encodes a protein with one of the two reading frames corresponding to PSA-EHTs1 (SEQ ID NO: 161), or PSA-EHTs2 (SEQ ID NO: 162). The mutation at position 54 in PSA-EHTs1 replaces a leucine residue with a proline residue.

PSA-EHTt (SEQ ID NO: 48):

This isoform exhibits retention of a truncated intron 4 (between positions 4727 and 5111). It can be seen that the nucleotides at position 137 and 239 of SEQ ID NO: 48 are G and A, whereas the Genbank reference sequence indicates A and G. These differences can be explained by the existence of a polymorphism at these positions or by errors in the referenced sequence, although a polymerase-induced mutation cannot be excluded. This isoform encodes a protein with one of the two reading frames corresponding to PSA-EHTt1 (SEQ ID NO: 163) or PSA-EHTt2 (SEQ ID NO: 164).

PSA-EHTu (SEQ ID NO: 49):

This isoform results from the use of a cryptic site in intron 4 (at position 5056). It can be seen that the nucleotide at position 48 de SEQ ID NO: 49 is T, whereas the Genbank reference sequence indicates C. This difference can be explained by the existence of a polymorphism at this position or by errors in the referenced sequence, although a polymerase-induced mutation cannot be excluded. PSA-EHTm encodes a protein with one of the three reading frames corresponding to PSA-EHTu1 (SEQ ID NO: 165), PSA-EHTu2 (SEQ ID NO: 166) or PSA-EHTu3 (SEQ ID NO: 167). Mutation 48 replaces the alanine residue with a valine residue in PSA-EHTu2.

### C—Validation of the Expression of the PSA and KLK2 Isoforms Using a Microarray of Junction Oligonucleotides

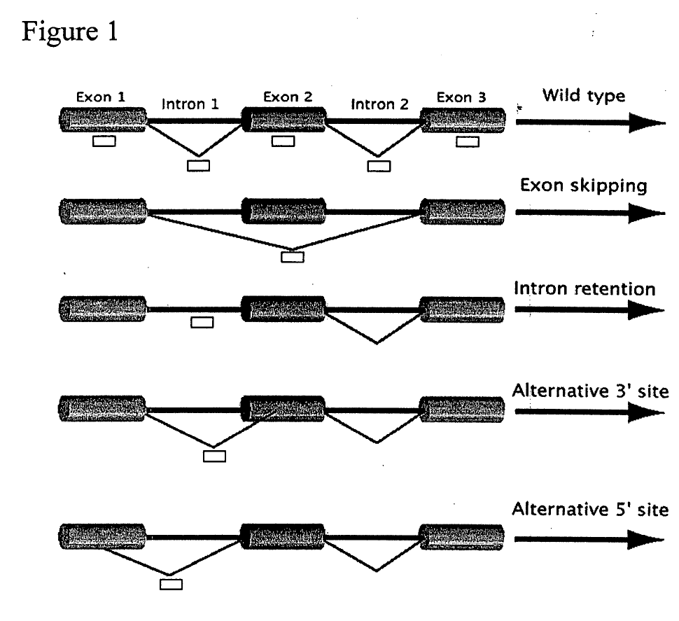

The expression of the PSA and KLK2 variants described in this invention was established using a microarray of oligonucleotides capable of hybridising specifically with these variants. Based on their sequences, the splice variants of PSA and klk2 arise from different types of events (FIG. 1).
“exon skipping”: the specific (e.g. discriminating) oligonucleotide is designed to be complementary to the sequence created by the exon1-exon3 junction
intron retention: The specific oligonucleotide is located in the intron sequence.
in cases where alternative 5′ or 3′ splice sites are used, the discriminating oligonucleotide is designed to be complementary to (i.e. is placed over) one of these new junctions.
oligonucleotides are also generated in the exons and on the junctions of wild-type forms of klk2 and PSA.
   C1—Description of the Microarray of Junction Oligonucleotides.

This study consisted of generating 149 oligonucleotides of 24- and 25-mers. Their sequences are shown in the appendix (Table 1). Additional “discriminating” oligonucleotides from the specific junctions created by the variants described are claimed (SEQ ID NO: 168 to SEQ ID NO: 220).

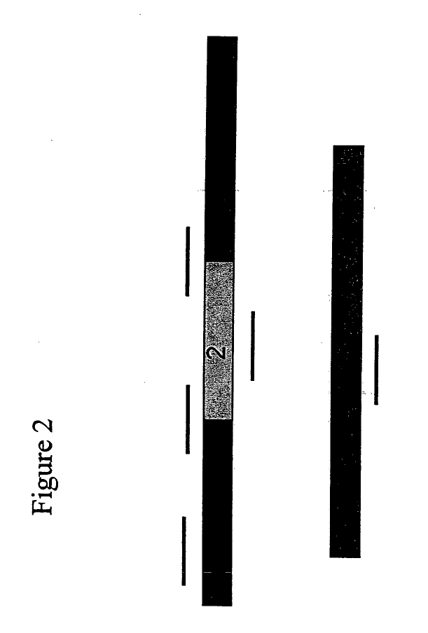

5 oligonucleotides were used to characterise each alternative splicing (see FIG. 2). One oligonucleotide is specific for the exon that is eliminated and enables quantification of the long form. A second oligonucleotide is specific for one of the flanking exons that is not involved in the splicing event and enables the long and short forms of the RNA to be quantified. Finally, three oligonucleotides are specific for the junctions; one of them is specific for the new sequence generated after splicing and enables the spliced form to be quantified. Of course, other combinations of oligonucleotide can be envisaged, notably the use of just one or two oligonucleotides.

Regarding the design of the oligonucleotides, given that the probes are shorter than the PCR product probes that are classically used, it is necessary to check that these probes do not hybridise in a non-specific manner to genes other than those for which they were designed. Furthermore, it is essential to make sure that the oligonucleotides have no secondary structure that could interfere with their ability to hybridise.

Generally, it is preferable for the chip if all the oligos generated have a uniform thermodynamic profile, namely in terms of Tm (65° C.) and length (24- or 25-mers). Furthermore, during their synthesis, the oligonucleotides can be modified by addition of a NH₂—C6 group to the 5′ end, promoting flexibility and enabling them to form a covalent bond with the polymer used to coat the glass slide.

Addressing the junction oligonucleotides more specifically, they should ideally be centred on the junctions, but we have also considered the possibility of oligonucleotides that are shifted with respect to the junction.

Primer Finder software was selected for designing the oligonucleotides. The criteria we selected are the following:
% GC: 40% to 60% for 24-mers and 30-mers, 30% to 60% for 40-mers.
Oligonucleotide concentrations: 50 nM
Salt concentration: 50 mM
Ignore oligonucleotides with a tendency to form “hairpin” secondary structures or homodimers.

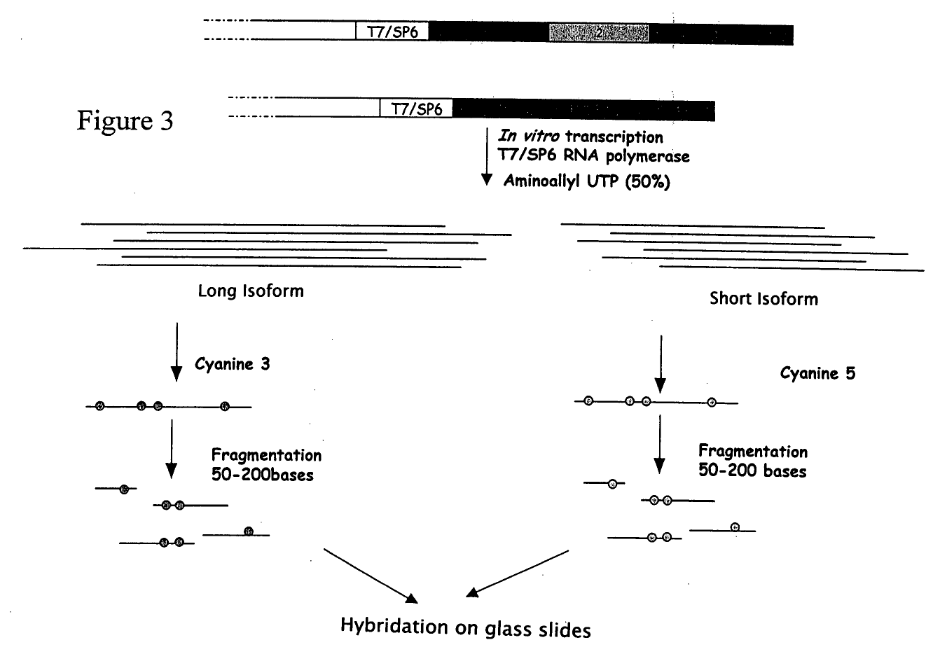

At first, we worked with cloned isoforms in order to validate our technology (see FIG. 3). Plasmids containing the long and short isoforms were linearised before in vitro transcription. The reaction medium contained aminoallyl-UTP, which forms a chemical interaction with the fluorochrome. We chose to label the long isoforms with Cy3 and the short isoforms with Cy5. As the RNA has a lot of secondary structure that could generate hybridisation, it was postulated that a chemical fragmentation step could be introduced into the protocol. After purification, the isoforms were mixed then hybridised on glass slides (3D Link, Motorola or Codelink, Amersham).

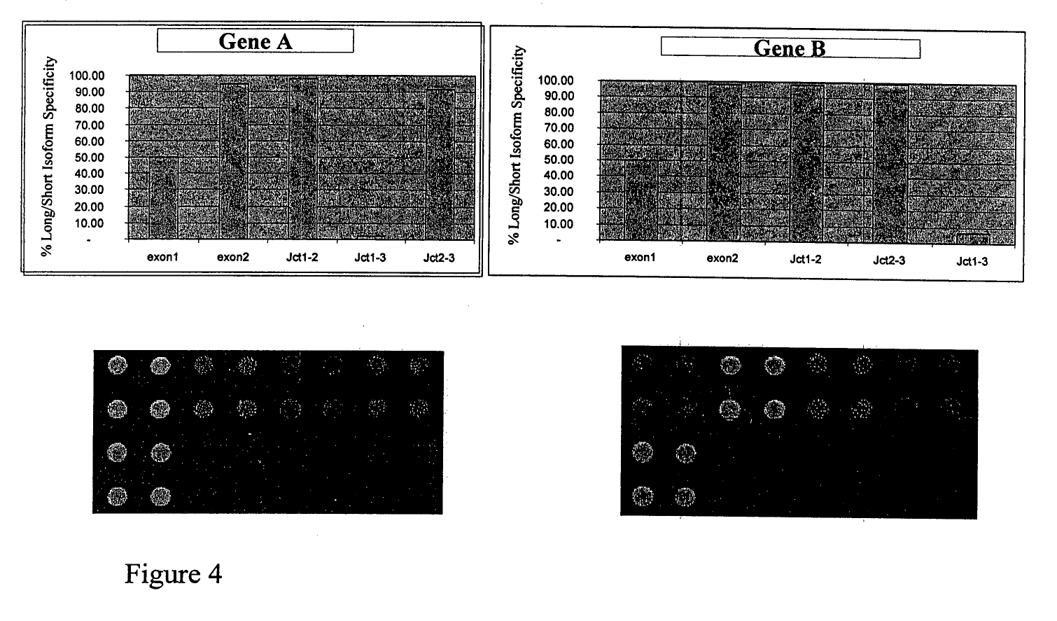

FIG. 4 shows the results obtained with two of the clones, corresponding to different genes. This experiment was performed in order to check the specificity of hybridisation of our oligonucleotides. We amplified drosophila RNA using in vitro transcription. An exogenous Arabidopsis thaliana control had been introduced into the RNA in order to calibrate the scanner for reading the slides. The labelled isoforms (5 ng per isoform) were then diluted in drosophila cRNA, which creates a complex environment, enabling us to check the specificity of hybridisation, given that drosophila RNA does not contain the sequence to which the target should hybridise (biocomputational analysis). After hybridisation, the slide was read in both the Cy3 and the Cy5 channels. The fluorescence intensity of each spot was measured and the values normalised by calculating the median.

Each oligonucleotide was spotted quadruplicate. The oligonucleotides corresponding to exon 2, to junctions 1-2 and 2-3 were designed only to hybridise to the long form, which is why they appear red on the image generated by QuantArray. The oligonucleotides specific for junction 1-3 are only supposed to hybridise to the short forms, and accordingly appear green. As an equimolar mixture of long and short isoforms was used, superimposition of both images shows orange spots. (Similar experiments have been performed to determine the sensitivity of our chip, by diluting the isoforms down to 26 pg).

This experiment shows that after normalisation, 50% of hybridisation was due to the long form if we consider the common exon. Between 90% and 100% of hybridisation was due to the long form if we consider exon 2, junctions 1-2 and 2-3. Less than 7% of the hybridisation was due to the long form for junction 1-3. These experiments validate the design of the oligos and the specificity of hybridisation. This high degree of specificity is crucial in order to be able to use this tool to quantify the isoforms.

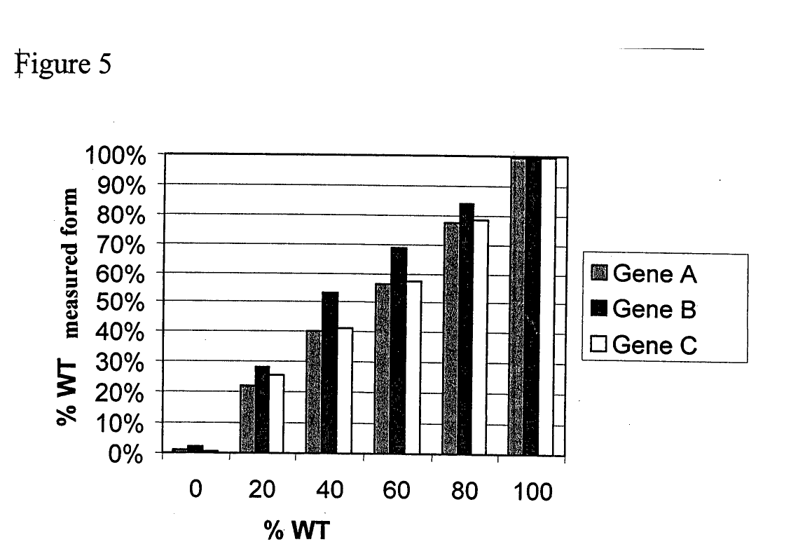

The previous results were obtained using an equimolar mixture of long and short isoforms. The aim of the next stage was therefore to show that the tool is quantitative (FIG. 5). In order to do this we varied the quantity of long forms in our samples from 0% to 100%, in increments of 20% (x-axis), the long and short forms being labelled with different fluorochromes.

After normalising the fluorescence intensities, we measured the % of long forms based on the values obtained on the common exon, which we plotted on the y-axis. As demonstrated in the graph, the measured values were very close to the theoretical values.

All these studies mean that we can expect to be able to use the oligoarray tool in order to define, both qualitatively and quantitatively, the expression of spliced exons or intron retention.

149 oligonucleotides (24- and 25-mers) were designed to make the microarray. These oligonucleotides were taken up at a concentration of 25 uM in 150 mM Sodium Phosphate buffer. The oligonucleotides were then loaded onto glass slides (Codelink, Amersham), and the slides were incubated in a humidified chamber in NaCl for 16 hours. Next, unused reactive sites were blocked using a solution of 50 mM ethanolamine, 0.1 M Tris, 0.1% SDS at pH 9. They were then washed in a solution of 4×SSC/0.1% SDS.

The targets were hybridised in a buffer of 5×SSC, 0.1% SDS, 0.1 mg/ml salmon sperm DNA, at a temperature of 50° C. for 16 hours. They were then washed using increasingly stringent washing conditions:
4×SSC to remove the cover slip
2×SSC/0.1% SDS during 5 minutes at 50° C.
0.2×SSC during 5 minutes at room temperature
0.1×SSC during 5 minutes at room temperature
   C2—Determination of the Hybridisation Capacity of the Oligonucleotides

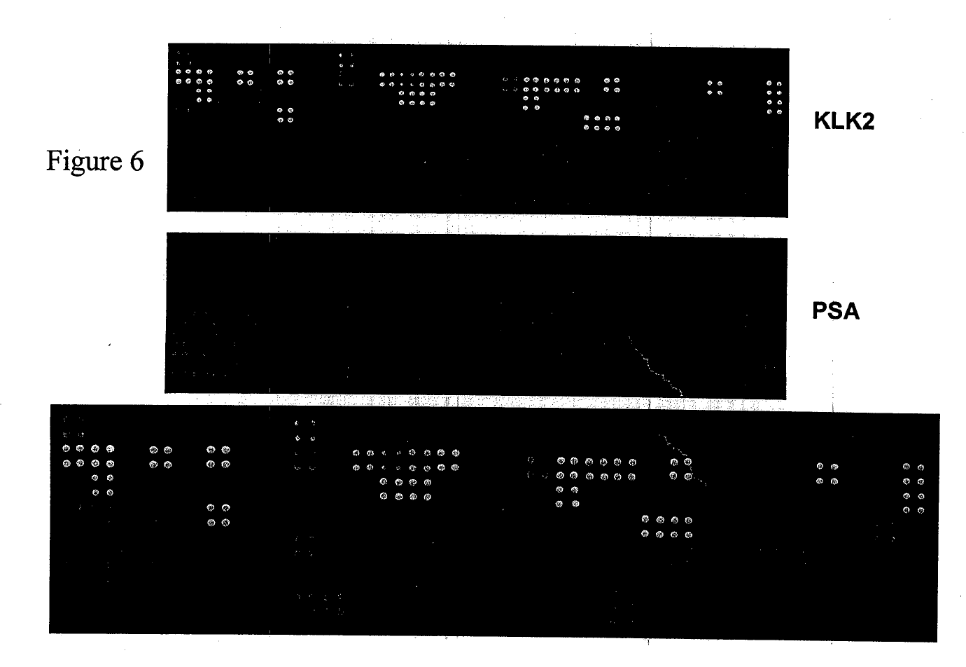

The hybridisation capacity and specificity of the oligonucleotides used to discriminate between PSA and klk2 were checked. In order to achieve this, we pooled several isoforms corresponding to klk2 that were labelled with cyanine 5 and several isoforms of PSA that were labelled with cyanine 3 (FIG. 6). The cRNAs were then cohybridised on a single slide that was read on 2 channels. When the 2 images were superimposed, it was revealed that there was no cross-hybridisation between the PSA and klk2 oligonucleotides, apart from one oligo that was applied in quadruplicate and subsequently redesigned.

C3—Studies on Neoplastic and Healthy Samples From Patients

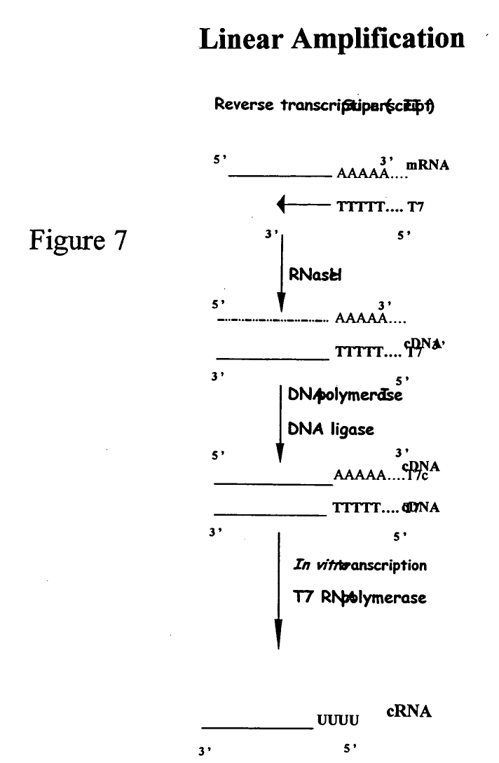

As there was usually insufficient biological material, we resorted to RNA amplification (FIG. 7). The first step consisted of reverse transcription of the mRNA in the presence of oligo-dT using Superscript II. The RNA that served as a template was degraded by Rnase H, leaving primers that can be used by DNA polymerase I for second strand cDNA synthesis. The synthesised fragments were assembled by DNA ligase. At the end of this step, a double-stranded DNA structure has been formed that is recognised by T7 DNA polymerase. This enzyme amplifies the strand corresponding to the sequence of the messenger and synthesises molecules of cRNA that hybridise to the probes of the complementary sequence (mRNA sense).

For each patient, 8 ug of target (corresponding to the neoplastic and healthy samples), labelled with 2 different fluorochromes were cohybridised on a single slide. The fluorescence intensities were measured in both channels and normalised using the global intensity method of the analysis software for reading the fluorescence of the glass slide (GeneTraffic).

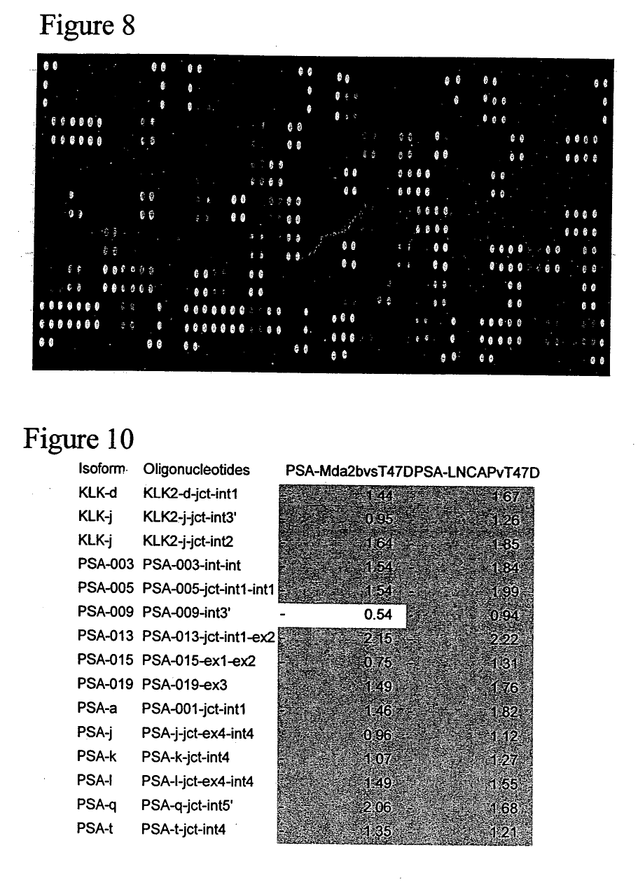

FIG. 8 shows two superimposed images obtained from one of the patients. Similar comments can be made for the three other patients analysed: the fluorescence signal is of high quality and the intensities are generally greater than 1,500.

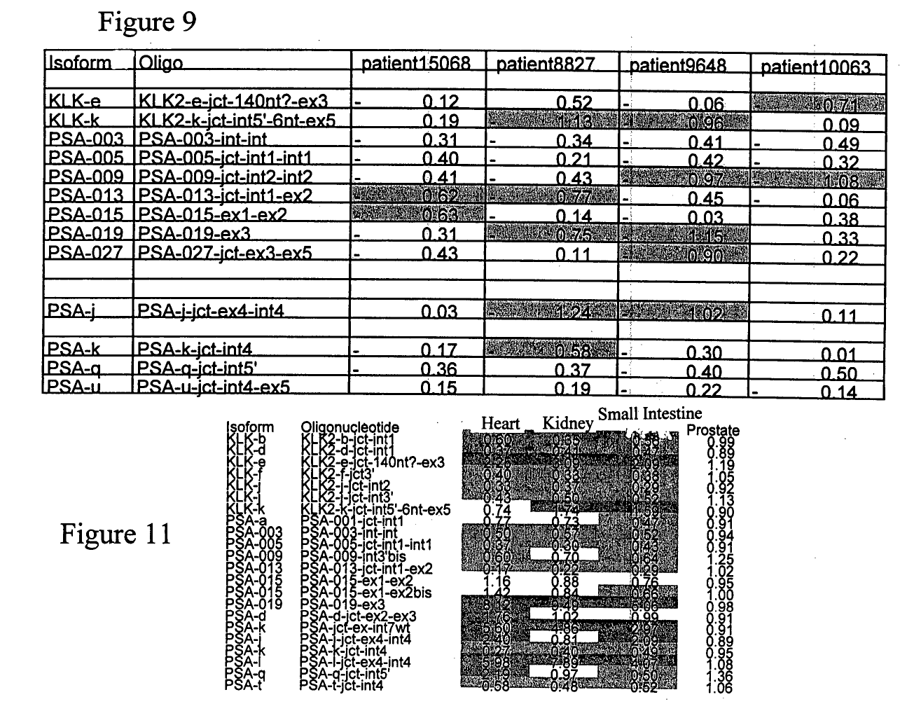

Analysis of the signals obtained from neoplastic and benign samples from the 4 patients demonstrated that some isoforms are expressed differentially in several patients (FIG. 9).

C4—Studies on Cell Lines Derived From Prostate Cancer and Breast Cancer

In some experiments, the expression profiles for PSA and KLK22 isoforms in prostate cancer and breast cancer cell lines were compared.

In order to do this, we amplified RNA from two prostate cancer cell lines (Mda2b and LnCAP) and 4 breast cancer cell lines (Mda231, T47D, Mcf7 and BT549). We then cohybridised each prostate cancer line with the various breast cancer lines, after having labelled the Mda2b and LnCAP lines with Cyanine 3 and the breast cancer lines with Cyanine 5.

The slides were read in both channels and the fluorescence intensities were normalised in GeneTraffic using the global intensity method. We divided the study into two hybridisation groups, one for each prostate cell line.

We then identified a list of discriminating oligonucleotides with deregulated expression in at least one hybridisation from a single hybridisation group. We selected oligos with a calculated ratio of less than 0.66 or greater than 1.5 (i.e. −0.58<Mean log2 ratio>0.58). We chose to present the results of the analyses of Mda2b versus T47D and LnCAP versus T47D, in which we observed the most marked differential expression involving the largest number of discriminating oligonucleotides.

Differential expression was observed for 15 isoforms, 3 of which (namely PSA-EHT019, PSA-EHTj and PSA-EHTl) were overexpressed in lines derived from prostate cancer compared to a breast cancer-derived line. The other 12 were underexpressed in prostate cancer (FIG. 10).

C5—Tissue Studies

These experiments consisted of checking the tissue-specific expression of PSA and KLK2 isoforms. In order to do this, we selected 4 tissues: the prostate, the heart, the kidney and the intestine. We amplified RNA from these 4 tissues and cohybridised Cyanine 3-labelled cRNA from the prostate with Cyanine 5-labelled cRNA from other tissues. We also cohybridised Cyanine 3-labelled cRNA from the prostate with Cyanine 5-labelled prostate cRNA.

The slides were read in both channels and the fluorescence intensities were normalised in GeneTraffic using the global intensity method.

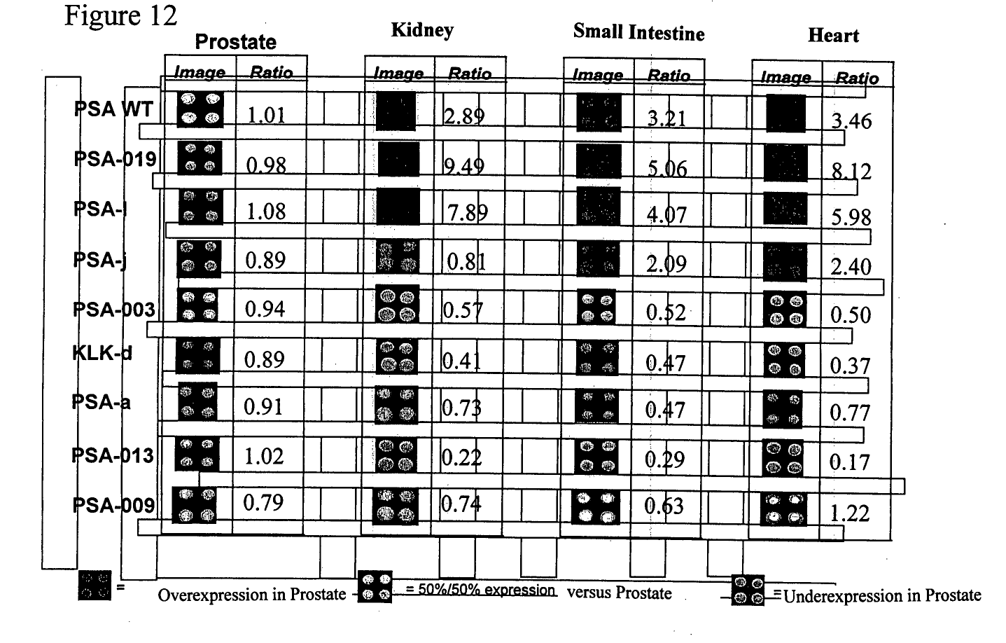

Next, we identified a list of discriminating oligonucleotides that had deregulated expression in at least one hybridisation within a hybridisation group of these 4 hybridisations. We selected oligonucleotides where the calculated ratio was less than 0.66 or greater than 1.5 (i.e. −0.58<Mean log2 ratio>0.58). We thereby showed deregulated expression for several PSA and KLK2 isoforms depending on the healthy tissue that was tested (prostate, heart, small intestine, kidney). These are: PSA-EHT003, PSA-EHT005, PSA-EHT013, klk2-EHTb, klk2-EHTd, klk2-EHTj klk2-EHTf and PSA-EHTl, PSA-EHT019 and klk2-EHTe (FIGS. 11 and 12).

C6—Summary of the Hybridisation Signals Obtained

Tables 3, 4 and 5 show the hybridisation signals obtained on the oligonucleotide microarray using healthy tissue (table 3), cell lines (table 4) and tissue from patients with prostate cancer (table 5). Values greater than twice the value of the background noise are indicated (representing significant hybridisation). Values of less than twice the background noise are represented by the abbreviation #NA. It appears that all discriminating oligonucleotides except the oligonucleotide SEQ ID NOs: 184, 215 and 220 produced significant signals in at least one of the systems studied. The expression of the isoforms described in this invention is therefore confirmed by this approach. It should be noted that the PSA-EHT 023 isoform that is associated with oligonucleotide SEQ ID NO: 184 was also detected using a more sensitive PCR approach (see section D, below). In conclusion, it appears that the majority of the isoforms described in the invention are actually expressed in one of the models studied. Tissue-specific and tumour-specific expression was also demonstrated.

### D—Validation of the Expression of PSA and KLK2 Isoforms by PCR

A PCR junction method was used to show the existence of some isoforms. The principle is based on specific amplification of isoforms using oligonucleotides specifically directed at the new junction resulting from the alternative splicing event already described. Amplification is performed using RNA from both benign and neoplastic areas from the prostate of each patient, and also using plasmid controls.

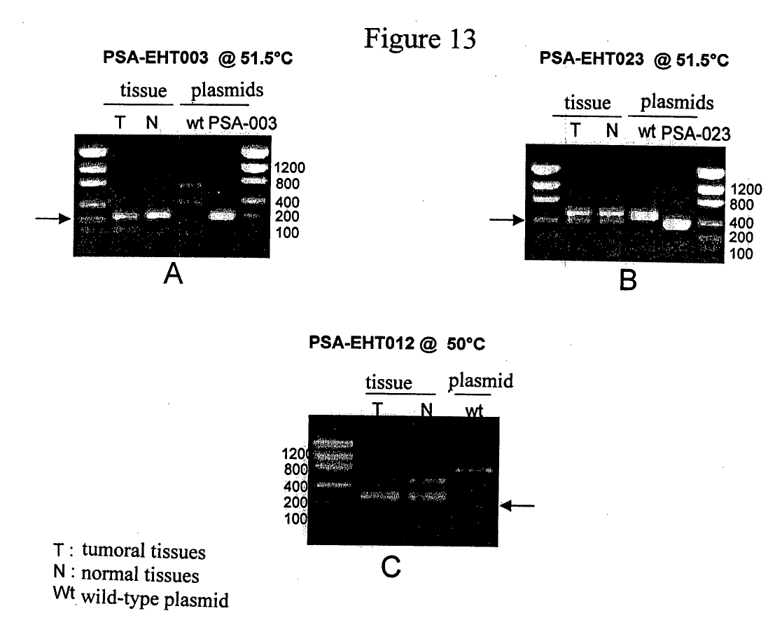

The PCR amplification results are shown in FIG. 13. The arrow indicates the band of the expected size. The desired result is specific amplification of the isoforms in the T (tumour) and N (normal) pools, with a negative wt control, i.e. no specific amplification of the size of the isoform when using the wild-type plasmid. The plasmid with the cloned isoform is used as a positive control for amplification.

| | Figure | |
|---|---|---|
| isoforms | # | conclusions |
| PSA-EHT003 | A | Amplicon of expected size and sequence using |
| | | the PSA-003 plasmid. |
| | | Non-specific amplification of wt plasmid that |
| | | does not correspond to the size of the isoform |
| | | expected. Positive amplification in both pools, |
| | | and only of the expected size for the isoform. |
| PSA-EHT023 | B | Amplicon of expected size and sequence using |
| | | the PSA-023 plasmid. |
| | | Non-specific amplification of wt plasmid that |
| | | does not correspond to the size of the isoform |
| | | expected. Positive amplification in both pools, |
| | | and only of the expected size for the isoform, |
| | | but also of the size obtained with the wt |
| | | plasmid. |
| PSA-EHT012 | C | Non-specific amplification of wt plasmid that |
| | | does not correspond to the size of the isoform |
| | | expected. Positive amplification in both pools, |
| | | of the expected size for the isoform. |

In conclusion, this method can also be used to demonstrate the presence of some isoforms in prostate tissue. PCR is more sensitive than the microarray technique, and it notably revealed the expression of PSA-EHT012.

### E—Antibody Production and Protein Expression

Polyclonal antibodies specific for some isoforms were produced in order to determine the existence of proteins encoded by some of the variants described in the invention. These antibodies were used in western blots to detect the expression of the corresponding protein.

Antibody Production and Protein Expression

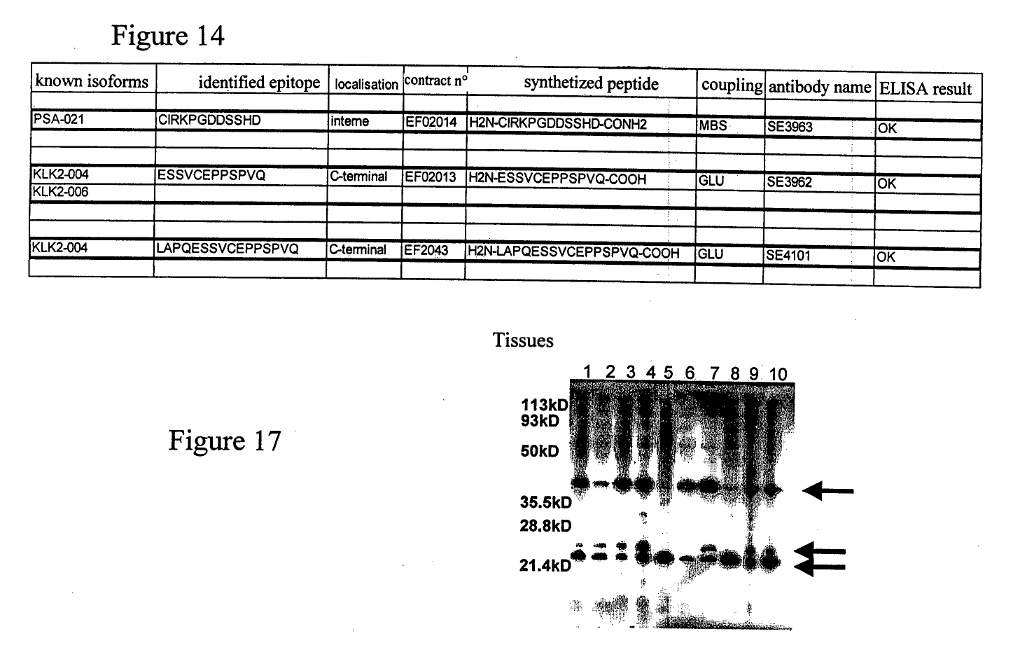

All the peptides and antibodies were produced by Eurogentec (Belgium). 20-30 milligrams of the peptides, corresponding to the sequences described in FIG. 14, were synthesised using Fmoc chemistry with a purity of over 70%. In order to induce an immune response, KLH was conjugated to 5 milligrams of each peptide using MBS (m-maleimidobenzoyl-N-hydroxysuccinimide ester) or glutaraldehyde.

Two rabbits (SPF New Zealand white rabbits) were immunised with 200 micrograms of conjugated peptide. The first injection was performed with Freund's complete adjuvant, whereas subsequent injections were performed in Freund's incomplete adjuvant. A standard protocol was used, comprising injections on days 0, 14, 28 and 56 and serum collection on days 0, 38 and 66. The final bleed took place on day 87.

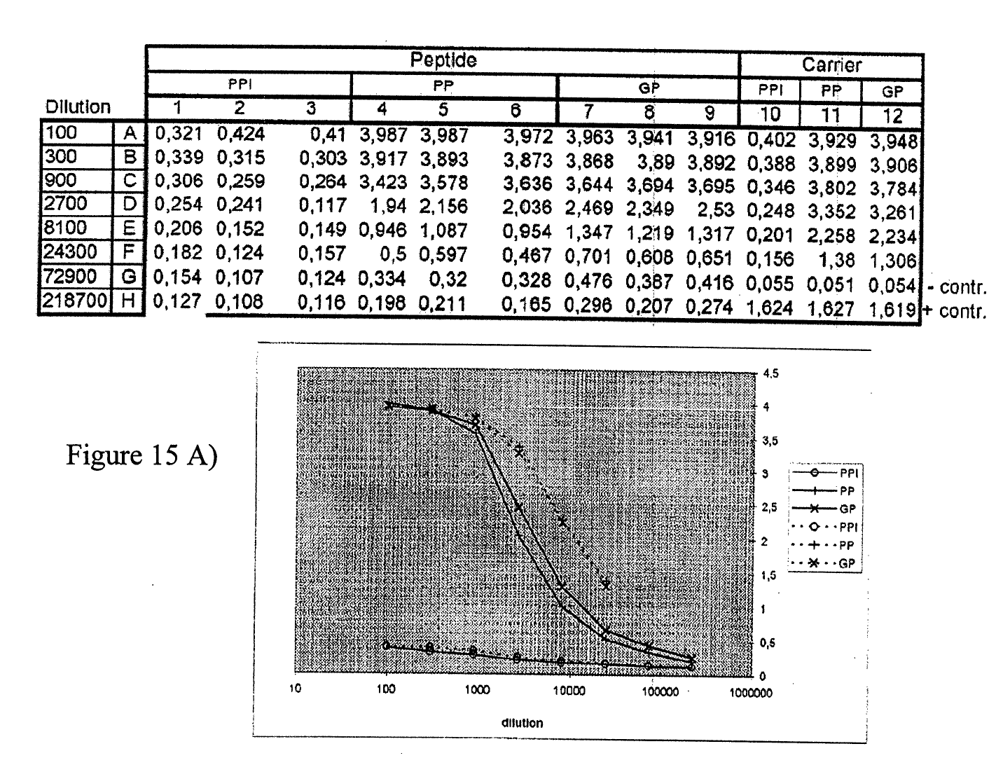

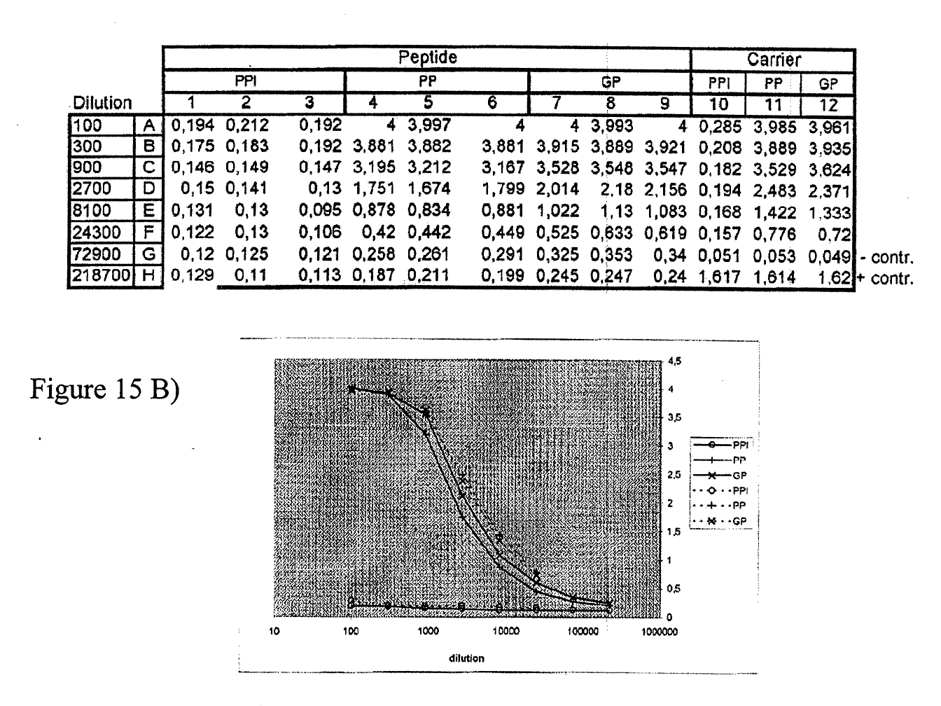

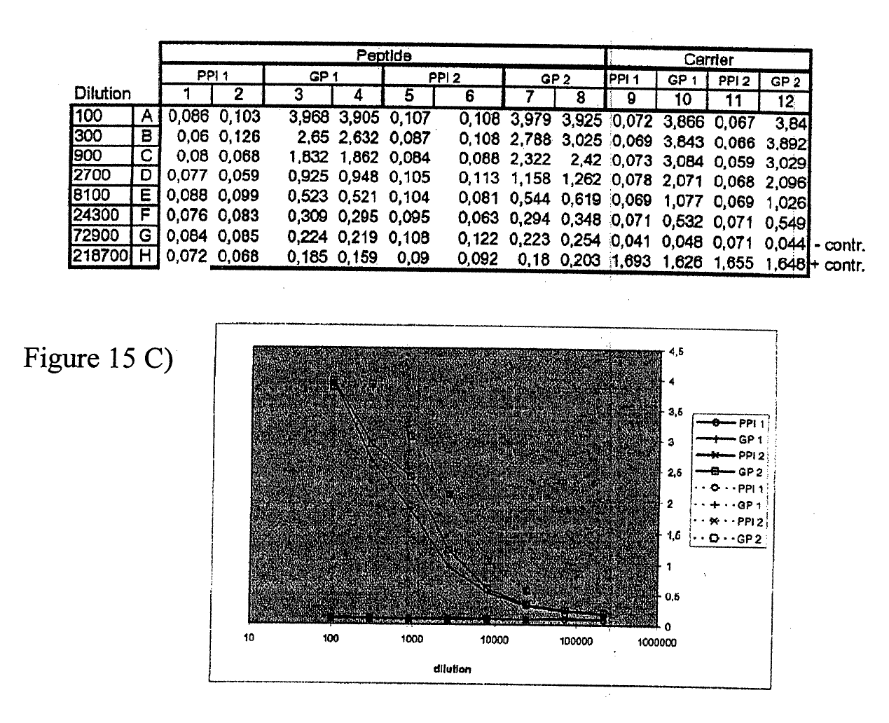

The antibody titre in the sera was measured by ELISA (FIG. 15). The antigens, synthetic peptides or KLH were loaded into the wells of ELISA plates (100 nanograms in PBS at 4° C. for 16 hours). After saturation (BSA 1 mg/ml at 25° C. for 2 hours), successive dilutions of the sera (preimmune: PPI, serum from the first harvest: PP and serum from the second harvest: GP) were incubated at 25° C. for 2 hours. The HRP/OPD system was used to show antibody binding, measuring the optical density at 492 nm. The titres obtained for the selected epitopes were satisfactory.

Western Blot Analysis

Protein extracts were prepared from tissues and cell lines using lysis buffer (50 mM Tris pH=7.5, 5 mM EGTA, 150 mM NaCl, 1 %, Triton 50 mM NaF, protease inhibitors (Roche)). Extracts were quantified using the Bradford method. When using tissue, 20 micrograms of extract were loaded onto a polyacrylamide-SDS gel. When using serum, 15 microlitres of a one-in-fifty dilution of non-purified serum or a one-in-eight dilution of purified serum (Aurum BioRad kit no 732-6701) were used.

After electrophoresis under denaturing conditions, the separated proteins were transferred onto a PVDF membrane. The PSA and KLK2 variants were then detected by incubation of the membrane with a specifically produced polyclonal antibody (see previous section). After washing, the membrane was incubated with a secondary anti-immunoglobulin antibody, labelled with peroxidase HRP (dilution 1/5000). The bands were then visualised using ECL detection (Amersham).

EHT- SE3962 Antibody

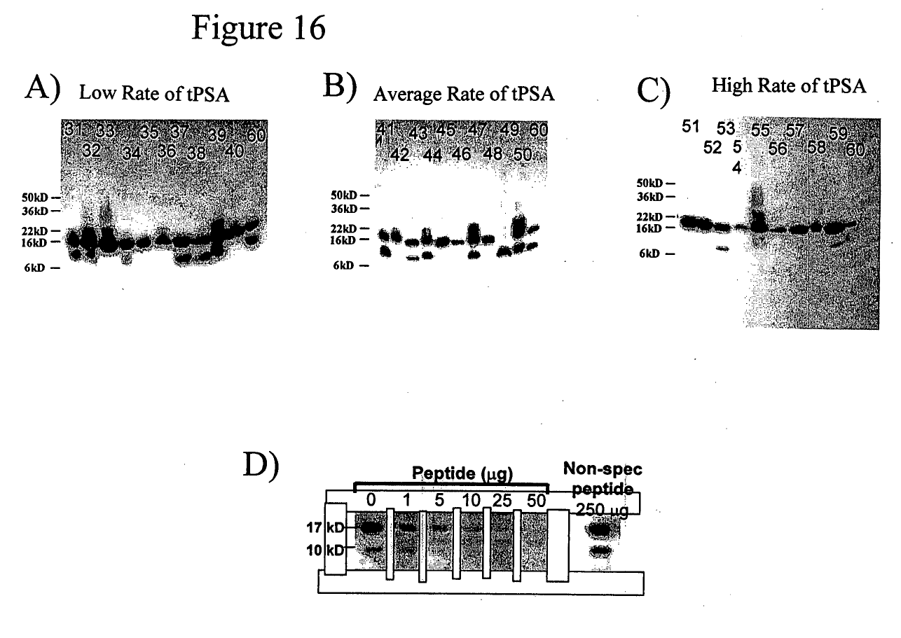

This antibody was generated from an epitope common to the KLK2-EHT004 and KLK2-EHT006 variants. The expected sizes for these two variants were 17 kD (KLK2-EHT006) and 10 kD (KLK2-EHT004). Two bands migrating at the expected sizes could be observed when using serum samples (FIG. 16). The antibody seems to recognise these bands specifically, because it was displaced by increasing doses of synthetic peptides corresponding to the chosen epitope (FIG. 16D). Heterogeneity was observed between the different serum samples. No obvious correlation was observed with the total PSA concentration (FIG. 16 A), B) and C))

EHT-SE3963 Antibody

This antibody was raised against a junction epitope corresponding to PSA-EHT021 (expected size: 20 kD). Three bands with approximate molecular weights of 22, 25 and 40 kD were observed using prostate tissue (FIG. 17). The band with the lowest molecular weight could correspond to PSA-EHT021. The 25 kD band could correspond to a variant that has already been described as having one of the two splicing events associated with PSA-EHT021 (Tanaka et al, 2000).

### References

David et al. (2002) J. Biol. Chem

Riegman et al (1988) Biochem. Biophys. Res. Commun. 155, 181-188.

Riegman et al (1991) Mol. Cell. Endicronol. 76,181-190.

Liu et al (1999) Biochem. Biophys. Res. Commun. 264, 833-839

Heuze et al. (1999) Cancer Res. 59, 2820-2824.

Heuzé-Vourc'h et al. (2001) Eur J Biochem. 268, 4408-4413.

Heuzé-Vourc'h et al. (2003) Eur J Biochem. 270, 706-714

Meng et al. (2002) Cancer Epidemiology, Biomarkers and Prevention 11, 305-309.

Tanaka et al (2000) Cancer Res. 60, 56-59.

Young et al. (1992) Biochemistry 31, 818-824.

TABLE 1

| | | SEQ |
|---|---|---|
| | | ID |
| Name: | Sequence (5′-3′) | NO: |
| PSA-exon1-wt | GTTGTCTTCCTCACCCTGTCCGTG | |
| PSA-exon2-wt | AGTGCGAGAAGCATTCCCAACCCT | |
| PSA-exon2bis-wt | AGGTGCTTGTGGCCTCTCGTGGCA | |
| PSA-exon3-wt | ACGATATGAGCCTCCTGAAGAATC | |
| PSA-exon4-wt | CTTGACCCCAAAGAAACTTCAGTG | |
| PSA-exon5-wt | AATGGTGTGCTTCAAGGTATCACG | |
| PSA-jctex1-2-wt | TGACGTGGATTGGCGCTGCGCCCC | |
| PSA-jctex2-3-wt | CTGCATCAGGAACAAAAGCGTGAT | |
| PSA-jctex3-4-wt | AACCAGAGGAGTTCTTGACCCCAA | |
| PSA-jctex4-5-wt | AGCACCTGCTCGGGTGATTCTGGG | |
| PSA-jct-ex-int1wt | TGACGTGGATTGGTGAGAGGGGCC | |
| PSA-jct-ex-int2wt | CCCCCTCTGCAGGCGCTGCGCCCC | |
| PSA-jct-ex-int3wt | CTGCATCAGGAAGTGAGTAGGGGC | |
| PSA-jct-ex-int4wt | CTTCCTCCCCAGCAAAAGCGTGAT | |
| PSA-jct-ex-int5wt | AACCAGAGGAGTGTACGCCTGGGC | |
| PSA-jct-ex-int6wt | CCTGGCCCGTAGTCTTGACCCCAA | |
| PSA-jct-ex-int7wt | AGCACCTGCTCGGTGAGTCATCCC | |
| PSA-jct-ex-int8wt | TTTTACCCTTAGGGTGATTCTGGG | |
| PSA-intron 1 | CTCTTTTCTGTCTCTCCCAGCCCC | |
| PSA-intron 2 | AGAGAGGGAAAGTTCTGGTTCAGG | |
| PSA-intron 2bis | GGGAGCGAAGTGGAGGATACAACC | |
| PSA-intron 4 | CCGTGTCTCATCTCATTCCCTCCT | |
| PSA-001-int-int | CCAGCACCCCAGCTCCCAGCTGCT | 168 |
| PSA-001-int3′ | CCAACCCTATCCCAGAGACCTTGA | |
| PSA-001-int3′bis | AGGATACCCAGATGCCAACCAGAC | |
| PSA-003-int-int | CCATACCCCCAGCCCCTCCCACTT | 169 |
| PSA-003-int3′ | GCCCCTCAATCCTATCACAGTCTA | |
| PSA-004-jctex1-int | GTGACGTGGATTGCTGTGAGTGTC | 170 |
| PSA-004intron1 | GACACCTCCTTCTTCCTAGCCAGG | |
| PSA-005-jct-int1-int1 | AGGCTCTTTCCCCCCAACCCTATC | 171 |
| PSA-008-jctex1-int | GTGACGTGGATTGGATACCCAGAT | 172 |
| PSA-009-jct-int2-int2 | TCCGCCTCTTATTCCATTCTTTCT | 173 |
| PSA-009-int3′ | GAGGCGCAGAGAAGGAGTGGTTCC | |
| PSA-009-int3′bis | GAGACACAGAGAAGGGCTGGTTCC | |
| PSA-010-jxt-ex1-ex2 | TGACGTGGATTGGTGCTGCACCCC | |
| PSA-0012-jctex2-int2 | GCATCAGGAATCTCCATATCCCCC | 174 |
| PSA-012-int3′ | TCACCTGTGCCTTCTCCCTACTGA | |
| PSA-013-jct-int1-ex1 | TGACGTGGATTGCACCCCCTCTGC | 175 |
| PSA-013-int3′ | GGCATTTTCCCCAGGATAACCTCT | |
| PSA-014-int3′ | GGACTGGGGGAGAGAGGGAAAGTT | |
| PSA-015-ex1-ex2 | GTCTTCCTCACCCTGAGCTTGTGG | 176 |
| PSA-015-ex1-ex2bis | CTTCCTCACCCTGAGCTTGTGGCC | 177 |
| PSA-016-ex1-ex2 | TGACGTGGATTGGGCAGTCTGCGG | 178 |
| PSA-018-jct-int2-int2 | GAGAAAAGAAAGGACCCTGGGGAG | 179 |
| PSA-018-jct-ex1-ex2 | TGACGTGGATTGGAGCTGCGCCCC | |
| PSA-018-int3′ | GAAGTGGAGGATACAACCTTGGGC | |
| PSA-019-ex3 | CAGTCTGTTTCATCCTGAAGACAC | |
| PSA-019-jct-ex4-5 | AGCACCTGCTGGGGTGATTCTGGG | |
| PSA-019-jct-ex3 | ATTTCAGGTCAGCCTGCCGAGATC | 180 |
| PSA-020-jct-ex3 | CTGCATCAGGAAGCCAGGTGATGA | |
| PSA-020-jxt-ex4-ex5 | AGCACCTGCTAGGGTGATTCTGGG | |
| PSA-020-ex3 | GTGATGACTCCAGCCACGACCTCA | |
| PSA-021-jct-ex3 | CTGCATCAGGAAGCCAGGTGATGA | 181 |
| PSA-021-jct-ex3-2 | GTGATGACTCCAGCATTGAACCAG | 182 |
| PSA-022-ex3 | TGATGACTCCAGCATTGAACCAGA | |
| PSA-023-jct-ex2 | GTCTCGGATTGTCTCTCGTGGCAG | 183 |
| PSA-023-jct-ex5 | AATGGGGTGCTTCAAGGTATCACG | |
| PSA-023-jct-in3-ex4 | CTGGGCCAGATGTCTTGACCCCAA | 184 |
| PSA-025-jct-ex2-ex4 | TGCATCAGGAATCTTGACCCCAAAG | 185 |
| PSA-026-jct-ex3 | TTGCTGGGTCAGCATTGAACCAGA | 186 |
| PSA-027-jct-ex3-ex5 | ATCTTGCTGGGTCGGGTGATTCTG | 187 |
| PSA-027-jct-ex3- | CTTGCTGGGTCGGGTGATTCTGGG | 188 |
| ex5bis | | |
| PSA-001-jct-int1 | CCAGCACCCCAGCTCCCTGCTCCC | |
| PSA-d-jct-ex2-ex3 | CTGCCCACTGCACCTGCTACGCCT | 189 |
| PSA-d-exon3 | GGGGCAGCATTGAACCAGAGGAGT | |
| PSA-f-jct-int5′ | TTGGTAACTGGCTTCGGTTGTGTC | |
| PSA-f-jct-int2 | CCCTCTCTTCTCTGTCTCACCTGTG | 190 |
| PSA-g-jct-ex2-int2 | CTGCATCAGGAATCTCCATATCTC | |
| PSA-g-jct-ex2-int2bis | GCATCAGGAATCTCCATATCTCCC | |
| PSA-h-jct-ex4-int4 | AGCACCTGCTCGGAGCTGGACCCT | 191 |
| PSA-h-jct3′ | GGAACTGCTATCTGTTATCTGCCTG | |
| PSA-h-exon5bis | TGTCTGTAATGGTGTGCTTCAAGG | |
| PSA-j-jct-ex4-int4 | AAGCACCTGCTCGTGGGTCATTCT | 192 |
| PSA-k-jct-ex4-int4 | CACCTGCTCGGTGAGTCATCCCTA | 193 |
| PSA-k-jct-int4 | GAGTCATCCCTACCCCTCTGTTGG | |
| PSA-l-jct-ex4-int4 | AGAAGGTGACCAAGTTCAGCACAC | 194 |
| PSA-l-jct-int3′ | AGGAACAGGGACCACAACACAGAA | |
| PSA-m-int1-5′ | GATGCTTGGCCTCCCAATCTTGCC | |
| PSA-m-jct-int1 | ACCCAGATGCCACCAGCCACCAAC | 195 |
| PSA-n-int1-5′ | GCCAACCAGACACCTCCTTCTTCC | |
| PSA-n-jct-int1 | CCTTAGGAAAAACATGAAGCCTCT | 196 |
| PSA-p-jct-ex1-int1 | GTGACGTGGATTGCCAGGCTATCT | 197 |
| PSA-q-jct-int5′ | CCAACTGGTGAAACCCCATCTCTA | |
| PSA-q-jct-int2 | AAAATTAGCCAGGCTACCTACCCA | 198 |
| PSA-r-jct-int2 | CCCTGAGAAAAGCCGCATCTACAG | 199 |
| PSA-r-jct-int3′ | CATCTACAGCTGAGCCACTCTGAG | |
| PSA-s-jct-int4 | GGTTATTCTTACAGCAGAGAGGAGG | 200 |
| PSA-s-jct-int3′ | GAGTCAGGAACTGTGGATGGTGCT | |
| PSA-t-jct-int5 | TGGGACATAGCAGTGAACAGACAG | |
| PSA-t-jct-int4 | GCTCTCAGGGAGGGCAGCAGGGAT | 201 |
| PSA-u-jct-int4-ex5 | GGCCTGGCTCAGGGTGATTCTGGG | 202 |
| KLK-2-exon1-wt | GTTCTCTCCATCGCCTTGTCTGTG | |
| KLK-2-exon2-wt | AGTGTGAGAAGCATTCCCAACCCT | |
| KLK-2-exon2bis-wt | GTACAGTCATGGATGGGCACACTG | |
| KLK-2-exon3-wt | CTGAAGCATCAAAGCCTTAGACCAG | |
| KLK-2-exon4-wt | CCAGGAGTCTTCAGTGTGTGAGCC | |
| KLK-2-exon5-wt | CACTTGTCTGTAATGGGGTGCTTC | |
| KLK2-jctex1-2-wt | TGGGGTGCACTGGTGCCGTGCCCC | |
| KLK2-jctex2-3-wt | ATTGCCTAAAGAAGAATAGCCAGG | |
| KLK2-jctex3-4-wt | AACCAGAGGAGTTCTTGCGCCCCA | |
| KLK2-jctex4-5-wt | AGACACTTGTGGGGGTGATTCTGG | |
| KLK2-intron1-wt | ACAGTTCAGCCCAGACAATGTGCC | |
| KLK2-intron2-wt | AGACACAGGGAGGGCTGGTTTCAG | |
| KLK2-intron3-wt | AGCCCAGTTTTTCTCTGACCCATA | |
| KLK2-intron4-wt | GGGAAGCAGCAGTGAACAGGTAGA | |
| KLK2-jct-ex-int1wt | TGGGGTGCACTGGTGAGATTGGGG | |
| KLK2-jct-ex-int2wt | CCCCCTCCGCAGGTGCCGTGCCCC | |
| KLK2-jct-ex-int3wt | TTGCCTAAAGAAGTAAGTAGGACC | |
| KLK2-jct-ex-int4wt | CTTCCTCCCCAGGAATAGCCAGGT | |
| KLK2-jct-ex-int6wt | TCTGACCCATAGTCTTGCGCCCCA | |
| KLK2-jct-ex-int7wt | GACACTTGTGGGGTGAGTCATCCC | |
| KLK2-jct-ex-int8wt | CTTTACCCTTAGGGTGATTCTGGG | |
| KLK2-002-jct-int2-ex3 | TCACTTCTCAGGAATAGCCAGGTC | 203 |
| KLK2-002-jct-ex3-ex4 | GATGTTGTGAAGGAGTCTTCAGTG | 204 |
| KLK2-002-ex4 | AGCCTCCATCTCCTGTCCAATGAC | |
| KLK2-003-exon5 | CACTTGTCTGTAATGGTGTGCTTC | |
| KLK2-003-jct-ex1-ex3 | TGGGGTGCACTGGAATAGCCAGGT | 205 |
| KLK2-003-jct-int4-ex5 | CTGGAGGGGAAAGGGTGATTCTGG | 206 |
| KLK2-004-jct-ex2-ex4 | TTGCCTAAAGAATCTTGCGCCCCA | 207 |
| KLK2-004-int4 | AACATCTGGAGGGGAAAAGTGAGT | |
| KLK2-005-int4 | AACATCTGGAGGGGAAAGGTGAGT | |
| KLK2-008-ex4 | ATCCTCCATCTCCTGTCCAATGAC | |
| KLK2-008-jct-ex3-ex4 | GAACCAGAGGAGTGAGTCTTCAGC | |
| KLK2-009-jct-ex3-ex4 | GAACCAGAGGAGTGAGTCTTCAGT | 208 |
| KLK2-009-jct-ex3 | TGAAGACTCCAGCATCGAACCAGA | 209 |
| KLK2-009-ex4 | CTTCAGTGTGTGAGCCTCCATCTC | |
| KLK2-011-jct-ex3-ex4 | AACCAGAGGAGTGGTAAAGACACT | 210 |
| KLK2-011-jct-ex4-int4 | AGACACTTGTGGGGTGAGTCATCC | |
| KLK2-a-exon3 | ATGAGCCTTCTGAAGCATCAAAGC | |
| KLK2-a-exon3bis | CCCACACCCGCTCTACAATATGAG | |
| KLK2-b-jct-int1 | CTGACTCTTCCCCCCGAGGCTATCT | 211 |
| KLK2-b-jct-int3′ | ACTCTTTGCCCCAGACCCGTCATT | |
| KLK2-c-jct-int1 | TGGGTGCACTGACCCGTCATTCA | 212 |
| KLK2-d-jct-int5′ | GCGGGTTCTGACTCTTATGCTGAA | |
| KLK2-d-jct-int1 | CAGCCTCGTCCCCCCAACCACAAC | 213 |
| KLK2-e-ex2 | CAGTCATGGATGGGCACACTGTGG | |
| KLK2-e-ex2-140nt? | TAGTGGAACCCTGCTATCTGCCGA | 214 |
| KLK2-e-jct-140nt?-ex3 | TTTTCTCAGGAATAGCCAGGTCTG | 215 |
| KLK2-f-jct-ex2-int2 | GATGGGCACACTCCTGTTTTCTAA | 216 |
| KLK2-f-jct3′ | CCTTTCCCCATTTTCTCTCTCCTC | |
| KLK2-g-ex5 | CACTTGTCTGTAATGGGTGCTTCA | |
| KLK2-g-int4 | AGTCATCCCTACTCCCAACATCTG | |
| KLK2-h-jct3′ | GAGTCTTCAGTGTGTGAGCCTCCA | |
| KLK2-h-jct3′bis | GTCCAATGACATGTGTGCTAGAGC | |
| KLK2-i-ex4 | ACAGGTGGTAAAGACACTTGTGGG | |
| KLK2-j-jct-int3′ | CTGCTACTCCACACTCCTCAGATG | |
| KLK2-j-jct-int2 | ACATCCCTCCACCCTCATGCCTCT | 217 |
| KLK2-k-jct-int5′ | AGTCTCTCCCCTCCACTCCATTCT | 218 |
| KLK2-k-jct-int5′-6nt- | CCTGCCGATGGCCCACTTGTCTGT | 219 |
| ex5 | | |
| KLK2-l-jct-int2-ex3 | CCCCAGCTGCAGGAATAGCCAGGT | 220 |

TABLE 2

| Isoforms | Pairs of oligonucleotides |
|---|---|
| KLK2-EHT002 | 249/166 |
| KLK2-EHT003 | 249/170 |
| KLK2-EHT004 | 249/174 |
| KLK2-EHT006 | 249/166 |
| KLK2-EHT007 | 249/174 |
| KLK2-EHT009 | 249/166 |
| KLK2-EHT011 | 249/174 |
| KLK2-EHTb | 163/213 |
| KLK2-EHTc | 163/213 |
| KLK2-EHTd | 163/213 |
| KLK2-EHTe | 167/172 |
| KLK2-EHTf | 167/172 |
| KLK2-EHTj | 214/215 |
| KLK2-EHTk | 218/170 |
| KLK2-EHTl | 221/172 |
| PSA-EHT001 | 247/203 |
| PSA-EHT003 | 247/203 |
| PSA-EHT004 | 247/203 |
| PSA-EHT005 | 247/203 |
| PSA-EHT007 | 247/203 |
| PSA-EHT008 | 247/203 |
| PSA-EHT009 | 247/207 |
| PSA-EHT012 | 247/205 |
| PSA-EHT013 | 247/176 |
| PSA-EHT015 | 247/176 |
| PSA-EHT016 | 247/176 |
| PSA-EHT018 | 247/207 |
| PSA-EHT019 | 247/248 |
| PSA-EHT021 | 247/248 |
| PSA-EHT022 | 247/182 |
| PSA-EHT023 | 247/182 |
| PSA-EHT025 | 247/248 |
| PSA-EHT026 | 247/248 |
| PSA-EHT027 | 247/182 |
| PSA-EHTa | 175/203 |
| PSA-EHTd | 179/178 |
| PSA-EHTf | 179/205 |
| PSA-EHTh | 181/182 |
| PSA-EHTj | 181/182 |
| PSA-EHTk | 181/182 |
| PSA-EHTl | 181/209 |
| PSA-EHTm | 200/201 |
| PSA-EHTn | 200/201 |
| PSA-EHTp | 202/203 |
| PSA-EHTq | 204/207 |
| PSA-EHTr | 204/207 |
| PSA-EHTs | 208/211 |
| PSA-EHTt | 208/211 |
| PSA-EHTu | 210/182 |

TABLE 3

| | | Prostate/Heart | | Prostate/Kidney | | Prostate/Prostate | | Prostate/small intestine | |
|---|---|---|---|---|---|---|---|---|---|
| | SEQ | LEX.E - | LEX.R | LEX.E - | LEX.R | LEX.E - | LEX.R | LEX.E - | LEX.R |
| Oligonucleotide | ID NO | BG | Norm. | BG | Norm. | BG | Norm. | BG | Norm. |
| PSA-exon1-wt | | 10136 | 9449 | 7214 | 7071 | 8719 | 7071 | 8437 | 9909 |
| PSA-exon2-wt | | 13504 | 2364 | 10925 | 1629 | 15466 | 1629 | 12303 | 4372 |
| PSA-exon2bis-wt | | 5494 | 2823 | 3518 | 3028 | 7242 | 3028 | 5181 | 4738 |
| PSA-exon3-wt | | 31201 | 3591 | 13479 | 2223 | 22239 | 2223 | 17496 | 4698 |
| PSA-exon4-wt | | #N/A | #N/A | 7992 | 804 | 9684 | 804 | 9986 | 2222 |
| PSA-exon5-wt | | 22907 | 1889 | 20521 | 1829 | 33673 | 1829 | 26242 | 5875 |
| PSA-jctex1-2-wt | | #N/A | #N/A | #N/A | #N/A | 1485 | #N/A | #N/A | #N/A |
| PSA-jctex2-3-wt | | #N/A | #N/A | #N/A | #N/A | 7965 | #N/A | #N/A | #N/A |
| PSA-jctex3-4-wt | | #N/A | #N/A | 19379 | 950 | 25253 | 950 | 23869 | 3585 |
| PSA-jctex4-5-wt | | 14547 | 4663 | 12590 | 3938 | 17187 | 3938 | 14546 | 7193 |
| PSA-jct-ex-int1wt | | 1027 | 1745 | 924 | 1398 | 807 | #N/A | #N/A | #N/A |
| PSA-jct-ex-int2wt | | #N/A | #N/A | 677 | 1804 | #N/A | #N/A | #N/A | #N/A |
| PSA-jct-ex-int3wt | | 1513 | 3753 | 1341 | 3075 | 1275 | 3075 | 1130 | 2026 |
| PSA-jct-ex-int4wt | | 1598 | 1817 | 1088 | 1573 | 842 | #N/A | #N/A | #N/A |
| PSA-jct-ex-int5wt | | #N/A | #N/A | #N/A | #N/A | 1087 | #N/A | #N/A | #N/A |
| PSA-jct-ex-int6wt | | #N/A | #N/A | 1010 | 786 | 943 | 786 | #N/A | #N/A |
| PSA-jct-ex-int7wt | | #N/A | #N/A | 3902 | 830 | 4030 | 830 | #N/A | #N/A |
| PSA-jct-ex-int8wt | | #N/A | #N/A | 1843 | 803 | 3146 | 803 | #N/A | #N/A |
| PSA-intron 1 | | 6634 | 15678 | 3658 | 6958 | 2399 | #N/A | 2476 | 4832 |
| PSA-intron 2 | | #N/A | #N/A | #N/A | #N/A | #N/A | #N/A | #N/A | #N/A |
| PSA-intron 2bis | | #N/A | #N/A | #N/A | #N/A | #N/A | #N/A | #N/A | #N/A |
| PSA-intron 4 | | #N/A | #N/A | 960 | 1214 | 786 | #N/A | #N/A | #N/A |
| PSA-001-int-int | 168 | 2092 | 1956 | 1747 | 2767 | 1212 | #N/A | 1221 | 1993 |
| PSA-001-int3′ | | #N/A | #N/A | #N/A | #N/A | 680 | #N/A | #N/A | #N/A |
| PSA-001-int3′bis | | 1337 | 1779 | 924 | 906 | 1060 | 906 | #N/A | #N/A |
| PSA-003-int-int | 169 | 18443 | 37922 | 13312 | 22526 | 9648 | #N/A | 9956 | 19286 |
| PSA-003-int3′ | | 2243 | 1764 | 1539 | 1457 | 1327 | 1457 | 1240 | 2643 |
| PSA-004-jctex1-int | 170 | #N/A | #N/A | 822 | 954 | 879 | #N/A | #N/A | #N/A |
| PSA-004-intron1 | | 6720 | 25324 | 3446 | 11026 | 2074 | 11026 | 2771 | 6330 |
| PSA-005-jct-int1-int1 | 171 | 7522 | 20800 | 4894 | 14936 | 2421 | #N/A | 2490 | 5722 |
| PSA-008-jctex1-int | 172 | #N/A | #N/A | #N/A | #N/A | 710 | #N/A | #N/A | #N/A |
| PSA-009-jct-int2-int2 | 173 | 17473 | 13828 | 10188 | 12716 | 9302 | #N/A | 12859 | 22063 |
| PSA-009-int3′ | | #N/A | #N/A | 680 | 899 | #N/A | 899 | #N/A | #N/A |
| PSA-009-int3′bis | | #N/A | #N/A | 863 | 1180 | 765 | #N/A | #N/A | #N/A |
| PSA-010-jxt-ex1-ex2 | | #N/A | #N/A | #N/A | #N/A | 2617 | #N/A | #N/A | #N/A |
| PSA-0012-jctex2-int2 | 174 | 791 | 2114 | 611 | 1908 | #N/A | 2189 | #N/A | #N/A |
| PSA-012-int3′ | | 47778 | 120575 | 17806 | 65227 | 18078 | 65227 | 22707 | 67915 |
| PSA-013-jct-int1-ex1 | 175 | 15653 | 95691 | 10657 | 48699 | 7936 | #N/A | 6554 | 22783 |
| PSA-013-int3′ | | #N/A | #N/A | #N/A | #N/A | 775 | #N/A | #N/A | #N/A |
| PSA-014-int3′ | | 1854 | 4464 | 1553 | 3710 | 1297 | 4652 | 1218 | 2355 |
| PSA-015-ex1-ex2 | 176 | 3217 | 2963 | 2402 | 2851 | 2569 | 2851 | 2184 | 2789 |
| PSA-015-ex1-ex2bis | 177 | 3252 | 2277 | 2463 | 3040 | 2085 | #N/A | 1657 | 2530 |
| PSA-016-ex1-ex2 | 178 | #N/A | #N/A | 882 | 727 | 1295 | 727 | #N/A | #N/A |
| PSA-018-jct-int2-int2 | 179 | #N/A | #N/A | 659 | 780 | #N/A | #N/A | #N/A | #N/A |
| PSA-018-jct-ex1-ex2 | | #N/A | #N/A | 1925 | 779 | 3457 | 779 | 2749 | 1876 |
| PSA-018-int3′ | | #N/A | #N/A | #N/A | #N/A | #N/A | #N/A | #N/A | #N/A |
| PSA-019-ex3 | | #N/A | #N/A | #N/A | #N/A | 6126 | #N/A | #N/A | #N/A |
| PSA-019-jct-ex4-5 | | 8830 | 3451 | 3594 | 2166 | 6349 | 2166 | 5830 | 2743 |
| PSA-019-jct-ex3 | 180 | #N/A | #N/A | 1443 | 726 | 1516 | 726 | #N/A | #N/A |
| PSA-020-jct-ex3 | | #N/A | #N/A | 1160 | 774 | 1187 | 774 | #N/A | #N/A |
| PSA-020-jxt-ex4-ex5 | | 5525 | 2045 | 4051 | 1696 | 6248 | 1696 | 5287 | 3130 |
| PSA-020-ex3 | | 15229 | 2166 | 8244 | 1921 | 11202 | 1921 | 9939 | 3147 |
| PSA-021-jct-ex3 | 181 | #N/A | #N/A | 1160 | 774 | 1187 | 774 | #N/A | #N/A |
| PSA-021-jct-ex3-2 | 182 | #N/A | #N/A | #N/A | #N/A | 1286 | #N/A | #N/A | #N/A |
| PSA-022-ex3 | | #N/A | #N/A | #N/A | #N/A | 965 | #N/A | #N/A | #N/A |
| PSA-023-jct-ex2 | 183 | #N/A | #N/A | 824 | 814 | 938 | 814 | #N/A | #N/A |
| PSA-023-jct-ex5 | | 22666 | 2959 | 5976 | 1541 | 17888 | 1541 | 13759 | 3341 |
| PSA-023-jct-in3-ex4 | 184 | #N/A | #N/A | #N/A | #N/A | 710 | #N/A | #N/A | #N/A |
| PSA-025-jct-ex2-ex4 | 185 | #N/A | #N/A | #N/A | #N/A | 1161 | #N/A | #N/A | #N/A |
| PSA-026-jct-ex3 | 186 | #N/A | #N/A | 1217 | 980 | 1218 | 980 | #N/A | #N/A |
| PSA-027-jct-ex3-ex5 | 187 | #N/A | #N/A | 2106 | 904 | 3105 | 904 | #N/A | #N/A |
| PSA-027-jct-ex3- | 188 | 3506 | 5270 | 2711 | 3814 | 4271 | 3814 | 3798 | 3896 |
| ex5bis | | | | | | | | | |
| PSA-001-jct-int1 | | 9313 | 12287 | 7218 | 9436 | 4266 | #N/A | 4167 | 9493 |
| PSA-d-jct-ex2-ex3 | 189 | 12726 | 7316 | 8521 | 8517 | 6923 | 8517 | 5764 | 5523 |
| PSA-d-exon3 | | 22046 | 2136 | 6998 | 1321 | 17960 | 1321 | 17581 | 3395 |
| PSA-f-jct-int5′ | | #N/A | #N/A | #N/A | #N/A | 585 | #N/A | #N/A | #N/A |
| PSA-f-jct-int2 | 190 | 2322 | 1741 | 1677 | 2099 | 1292 | #N/A | 1406 | 3352 |
| PSA-g-jct-ex2-int2 | | #N/A | #N/A | #N/A | #N/A | #N/A | #N/A | #N/A | #N/A |
| PSA-g-jct-ex2-int2bis | | #N/A | #N/A | #N/A | #N/A | #N/A | #N/A | #N/A | #N/A |
| PSA-h-jct-ex4-int4 | 191 | #N/A | #N/A | #N/A | #N/A | 1412 | #N/A | #N/A | #N/A |
| PSA-h-jct3′ | | #N/A | #N/A | 2564 | 674 | 2855 | 674 | #N/A | #N/A |
| PSA-h-exon5bis | | 20025 | 1950 | 12838 | 1304 | 21421 | 1304 | 12938 | 2485 |
| PSA-j-jct-ex4-int4 | 192 | 10828 | 4375 | 3320 | 4091 | 5871 | #N/A | 5376 | 2563 |
| PSA-k-jct-ex4-int4 | 193 | #N/A | #N/A | #N/A | #N/A | 830 | #N/A | #N/A | #N/A |
| PSA-k-jct-int4 | | 7596 | 28023 | 5453 | 14512 | 3723 | #N/A | 3299 | 6639 |
| PSA-l-jct-ex4-int4 | 194 | #N/A | #N/A | 4924 | 700 | 5261 | 700 | #N/A | #N/A |
| PSA-l-jct-int3′ | | #N/A | #N/A | 770 | 787 | 726 | #N/A | #N/A | #N/A |
| PSA-m-int1-5′ | | 15735 | 17573 | 6677 | 9630 | 9745 | 9630 | 14172 | 29222 |
| PSA-m-jct-int1 | 195 | 2687 | 2869 | 2018 | 2678 | 1242 | #N/A | 1130 | 1896 |
| PSA-n-int1-5′ | | 5205 | 23386 | 3569 | 9307 | 2014 | #N/A | 2150 | 4770 |
| PSA-n-jct-int1 | 196 | #N/A | #N/A | #N/A | #N/A | #N/A | #N/A | #N/A | #N/A |
| PSA-p-jct-ex1-int1 | 197 | N/A | #N/A | #N/A | #N/A | #N/A | #N/A | #N/A | #N/A |
| PSA-q-jct-int5′ | | 54860 | 24878 | 56601 | 58160 | 53021 | #N/A | 53040 | 107366 |
| PSA-q-jct-int2 | 198 | #N/A | #N/A | #N/A | #N/A | #N/A | #N/A | #N/A | #N/A |
| PSA-r-jct-int2 | 199 | #N/A | #N/A | #N/A | #N/A | #N/A | #N/A | #N/A | #N/A |
| PSA-r-jct-int3′ | | #N/A | #N/A | #N/A | #N/A | #N/A | #N/A | #N/A | #N/A |
| PSA-s-jct-int4 | 200 | 1297 | 2262 | 1276 | 2275 | 1138 | #N/A | 1026 | 1905 |
| PSA-s-jct-int3′ | | #N/A | #N/A | 895 | 825 | 1021 | 825 | #N/A | #N/A |
| PSA-t-jct-int5′ | | #N/A | #N/A | 681 | 643 | 688 | 643 | #N/A | #N/A |
| PSA-t-jct-int4 | 201 | 1750 | 3107 | 1497 | 2929 | 1155 | 2929 | 1403 | 2632 |
| PSA-u-jct-int4-ex5 | 202 | 2534 | 1898 | 2314 | 1804 | 4007 | 1804 | 2913 | 2837 |
| KLK-2-exon1-wt | | 3774 | 1867 | 2501 | 1499 | 3298 | 1499 | 2533 | 2440 |
| KLK-2-exon2-wt | | 20150 | 3566 | 9082 | 1632 | 14005 | 1632 | 11966 | 4616 |
| KLK-2-exon2bis-wt | | #N/A | #N/A | 2062 | 850 | 3789 | 850 | 3381 | 1878 |
| KLK-2-exon3-wt | | #N/A | #N/A | 9886 | 722 | 10309 | 722 | 9742 | 1784 |
| KLK-2-exon4-wt | | #N/A | #N/A | 2337 | 755 | 3191 | 755 | #N/A | #N/A |
| KLK-2-exon5-wt | | 5472 | 1665 | 2236 | 857 | 7166 | 857 | 6254 | 1931 |
| KLK2-jctex1-2-wt | | #N/A | #N/A | #N/A | #N/A | 1142 | #N/A | #N/A | #N/A |
| KLK2-jctex2-3-wt | | #N/A | #N/A | #N/A | #N/A | 4569 | #N/A | #N/A | #N/A |
| KLK2-jctex3-4-wt | | #N/A | #N/A | 14928 | 1179 | 18564 | 1179 | 13927 | 2078 |
| KLK2-jctex4-5-wt | | 4945 | 1842 | 1999 | 927 | 5223 | 927 | 3967 | 2031 |
| KLK2-intron1-wt | | #N/A | #N/A | #N/A | #N/A | #N/A | #N/A | #N/A | #N/A |
| KLK2-intron2-wt | | 1684 | 3126 | 1199 | 1912 | 1017 | #N/A | 1181 | 2105 |
| KLK2-intron3-wt | | #N/A | #N/A | #N/A | #N/A | 682 | #N/A | #N/A | #N/A |
| KLK2-intron4-wt | | 1321 | 1875 | 1058 | 1536 | 1121 | #N/A | #N/A | #N/A |
| KLK2-jct-ex-int1wt | | 1120 | 2973 | 874 | 1844 | 1165 | 1844 | 997 | 1748 |
| KLK2-jct-ex-int2wt | | 2065 | 2380 | 1441 | 2309 | 1399 | #N/A | 1157 | 1741 |
| KLK2-jct-ex-int3wt | | #N/A | #N/A | #N/A | #N/A | #N/A | #N/A | #N/A | #N/A |
| KLK2-jct-ex-int4wt | | 1403 | 1869 | 796 | 1194 | 771 | #N/A | #N/A | #N/A |
| KLK2-jct-ex-int6wt | | #N/A | #N/A | 842 | 758 | 996 | 758 | #N/A | #N/A |
| KLK2-jct-ex-int7wt | | #N/A | #N/A | #N/A | #N/A | 1530 | #N/A | #N/A | #N/A |
| KLK2-jct-ex-int8wt | | #N/A | #N/A | 2712 | 760 | 3004 | 760 | #N/A | #N/A |
| KLK2-002-jct-int2-ex3 | 203 | #N/A | #N/A | #N/A | #N/A | #N/A | #N/A | #N/A | #N/A |
| KLK2-002-jct-ex3-ex4 | 204 | #N/A | #N/A | 614 | 1601 | #N/A | 1601 | #N/A | #N/A |
| KLK2-002-ex4 | | 10470 | 9254 | 6981 | 8205 | 7849 | #N/A | 7018 | 6964 |
| KLK2-003-exon5 | | 16595 | 1783 | 8520 | 1108 | 14895 | 1108 | 10729 | 2464 |
| KLK2-003-jct-ex1-ex3 | 205 | 1160 | 1850 | 920 | 1888 | 896 | #N/A | #N/A | #N/A |
| KLK2-003-jct-int4-ex5 | 206 | 1820 | 3246 | 1692 | 2851 | 1820 | 2851 | 1738 | 1865 |
| KLK2-004-jct-ex2-ex4 | 207 | #N/A | #N/A | #N/A | #N/A | 729 | #N/A | #N/A | #N/A |
| KLK2-004-int4 | | #N/A | #N/A | 734 | 819 | 767 | #N/A | #N/A | #N/A |
| KLK2-005-int4 | | 1386 | 2059 | 861 | 942 | 1051 | #N/A | #N/A | #N/A |
| KLK2-008-ex4 | | 4897 | 3279 | 3015 | 2005 | 4547 | 2005 | 5027 | 2829 |
| KLK2-008-jct-ex3-ex4 | | #N/A | #N/A | #N/A | #N/A | #N/A | #N/A | #N/A | #N/A |
| KLK2-009-jct-ex3-ex4 | 208 | #N/A | #N/A | #N/A | #N/A | #N/A | #N/A | #N/A | #N/A |
| KLK2-009-jct-ex3 | 209 | #N/A | #N/A | #N/A | #N/A | 1229 | #N/A | #N/A | #N/A |
| KLK2-009-ex4 | | #N/A | #N/A | #N/A | #N/A | 1330 | #N/A | #N/A | #N/A |
| KLK2-011-jct-ex3-ex4 | 210 | #N/A | #N/A | #N/A | #N/A | #N/A | #N/A | #N/A | #N/A |
| KLK2-011-jct-ex4-int4 | | #N/A | #N/A | 1503 | 713 | 2217 | 713 | #N/A | #N/A |
| KLK2-a-exon3 | | #N/A | #N/A | #N/A | #N/A | 6128 | #N/A | #N/A | #N/A |
| KLK2-a-exon3bis | | 17813 | 16492 | 13034 | 18648 | 21470 | 18648 | 22191 | 7763 |
| KLK2-b-jct-int1 | 211 | 3082 | 4931 | 2105 | 6012 | 1217 | 6012 | 1127 | 2007 |
| KLK2-b-jct-int3′ | | 2371 | 7491 | 1480 | 2886 | 1059 | #N/A | 1003 | 1717 |
| KLK2-c-jct-int1 | 212 | #N/A | #N/A | 620 | 644 | #N/A | #N/A | #N/A | #N/A |
| KLK2-d-jct-int5′ | | #N/A | #N/A | #N/A | #N/A | #N/A | #N/A | #N/A | #N/A |
| KLK2-d-jct-int1 | 213 | 8093 | 21205 | 5472 | 13180 | 3552 | 13180 | 3782 | 8081 |
| KLK2-e-ex2 | | 3721 | 1826 | 2689 | 1113 | 4220 | 1113 | 3118 | 1757 |
| KLK2-e-ex2-140nt? | 214 | #N/A | #N/A | 754 | 730 | 713 | 730 | #N/A | #N/A |
| KLK2-e-jct-140nt?- | 215 | #N/A | #N/A | #N/A | #N/A | 1804 | #N/A | #N/A | #N/A |
| ex3 | | | | | | | | | |
| KLK2-f-jct-ex2-int2 | 216 | #N/A | #N/A | 891 | 856 | 777 | 856 | #N/A | #N/A |
| KLK2-f-jct3′ | | 2008 | 4747 | 1081 | 3068 | 798 | #N/A | 882 | 2508 |
| KLK2-g-ex5 | | 12379 | 2989 | 3942 | 1765 | 11122 | 1765 | 11106 | 3415 |
| KLK2-g-int4 | | 3853 | 3369 | 2138 | 2068 | 2778 | 2068 | 2573 | 1832 |
| KLK2-h-jct3′ | | #N/A | #N/A | 5396 | 1209 | 7870 | 1209 | 6679 | 1692 |
| KLK2-h-jct3′bis | | #N/A | #N/A | #N/A | #N/A | 8277 | #N/A | #N/A | #N/A |
| KLK2-i-ex4 | | #N/A | #N/A | #N/A | #N/A | 3971 | #N/A | #N/A | #N/A |
| KLK2-j-jct-int3′ | | 1630 | 3787 | 841 | 1623 | 807 | 1623 | #N/A | #N/A |
| KLK2-j-jct-int2 | 217 | 10127 | 31847 | 5790 | 16169 | 3866 | 16169 | 3556 | 12265 |
| KLK2-k-jct-int5′ | 218 | 7637 | 9184 | 5059 | 8653 | 3328 | #N/A | 3431 | 6262 |
| KLK2-k-jct-int5′-6nt- | 219 | 6410 | 8657 | 2982 | 1814 | 7068 | 1814 | 4115 | 2751 |
| ex5 | | | | | | | | | |
| KLK2-l-jct-int2-ex3 | 220 | #N/A | #N/A | #N/A | #N/A | #N/A | #N/A | #N/A | #N/A |

TABLE 4

| | | PSA-Mda2bvBT549 | | PSA-Mda2bvMcf7 | | PSAMda2bvMda231 | | PSA-Mda2bvT47D | |
|---|---|---|---|---|---|---|---|---|---|
| | SEQ | LEX.E - | LEX.R | LEX.E - | LEX.R | LEX.E - | LEX.R | LEX.E - | LEX.R |
| Oligonucleotide | ID NO | BG | Norm. | BG | Norm. | BG | Norm. | BG | Norm. |
| PSA-exon1-wt | | 2320 | 4154 | 6065 | 7928 | 7080 | 9202 | 12711 | 19864 |
| PSA-exon2-wt | | 3143 | 1508 | 10772 | 4176 | 11062 | 5691 | 13314 | 6169 |
| PSA-exon2bis-wt | | 1429 | 2836 | 5771 | 7314 | 6065 | 8228 | 7725 | 9444 |
| PSA-exon3-wt | | 5517 | 2617 | 20290 | 7155 | 26160 | 8914 | 36486 | 13025 |
| PSA-exon4-wt | | 3726 | 1282 | 10419 | 2975 | 10145 | 2911 | 15418 | 5159 |
| PSA-exon5-wt | | 7179 | 2515 | 27572 | 7422 | 25909 | 8475 | 29812 | 9715 |
| PSA-jctex1-2-wt | | #N/A | #N/A | 1098 | 1069 | 2732 | 1148 | 1187 | 1203 |
| PSA-jctex2-3-wt | | 2439 | 1116 | 9486 | 3247 | 9972 | 4105 | 12558 | 4693 |
| PSA-jctex3-4-wt | | 6978 | 1454 | 18534 | 3273 | 20632 | 4338 | 25548 | 5645 |
| PSA-jctex4-5-wt | | 3772 | 2871 | 13605 | 8888 | 13291 | 8859 | 16245 | 12156 |
| PSA-jct-ex-int1wt | | #N/A | #N/A | 899 | 1362 | 2812 | 2264 | 1273 | 2551 |
| PSA-jct-ex-int2wt | | 1493 | 903 | #N/A | #N/A | #N/A | #N/A | #N/A | #N/A |
| PSA-jct-ex-int3wt | | #N/A | #N/A | #N/A | #N/A | #N/A | #N/A | #N/A | #N/A |
| PSA-jct-ex-int4wt | | #N/A | #N/A | 925 | 1173 | #N/A | #N/A | #N/A | #N/A |
| PSA-jct-ex-int5wt | | #N/A | #N/A | 1350 | 1078 | 1913 | 1383 | 1581 | 1447 |
| PSA-jct-ex-int6wt | | 1851 | 1126 | 1739 | 1525 | #N/A | #N/A | 3752 | 1933 |
| PSA-jct-ex-int7wt | | #N/A | #N/A | 1197 | 1049 | 2280 | 886 | 1578 | 1109 |
| PSA-jct-ex-int8wt | | #N/A | #N/A | 1923 | 1811 | 2003 | 1801 | 2396 | 2287 |
| PSA-intron 1 | | #N/A | #N/A | 958 | 2411 | 2531 | 4361 | 3067 | 1730 |
| PSA-intron 2 | | 956 | 1175 | 3578 | 1385 | #N/A | #N/A | 1354 | 1186 |
| PSA-intron 2bis | | #N/A | #N/A | #N/A | #N/A | #N/A | #N/A | #N/A | #N/A |
| PSA-intron 4 | | #N/A | #N/A | 828 | 1001 | #N/A | #N/A | #N/A | #N/A |
| PSA-001-int-int | 168 | #N/A | #N/A | 3201 | 1988 | #N/A | #N/A | #N/A | #N/A |
| PSA-001-int3′ | | #N/A | #N/A | #N/A | #N/A | #N/A | #N/A | 2679 | 2594 |
| PSA-001-int3′bis | | #N/A | #N/A | 1046 | 1053 | #N/A | #N/A | 1214 | 1374 |
| PSA-003-int-int | 169 | 975 | 4292 | 2609 | 8642 | 3883 | 10966 | 4673 | 14015 |
| PSA-003-int3′ | | #N/A | #N/A | 1088 | 5502 | 4148 | 3522 | #N/A | #N/A |
| PSA-004-jctex1-int | 170 | 2719 | 835 | 1700 | 1597 | #N/A | #N/A | #N/A | #N/A |
| PSA-004intron1 | | #N/A | #N/A | 938 | 1444 | #N/A | #N/A | #N/A | #N/A |
| PSA-005-jct-int1-int1 | 171 | 783 | 1797 | 1277 | 3597 | 1658 | 4080 | 1755 | 4762 |
| PSA-008-jctex1-int | 172 | #N/A | #N/A | #N/A | #N/A | 6670 | 1193 | #N/A | #N/A |
| PSA-009-jct-int2-int2 | 173 | 820 | 3116 | 2969 | 11450 | 3424 | 10694 | 3652 | 14112 |
| PSA-009-int3′ | | #N/A | #N/A | 966 | 1082 | #N/A | #N/A | #N/A | #N/A |
| PSA-009-int3′bis | | #N/A | #N/A | #N/A | #N/A | #N/A | #N/A | #N/A | #N/A |
| PSA-010-jxt-ex1-ex2 | | 1562 | 1415 | 3570 | 2493 | 6301 | 5459 | 7767 | 6232 |
| PSA-0012-jctex2-int2 | 174 | 2983 | 1050 | 2417 | 2332 | #N/A | #N/A | #N/A | #N/A |
| PSA-012-int3′ | | 1112 | 4129 | 1830 | 12147 | 2939 | 13399 | 2138 | 5740 |
| PSA-013-jct-int1-ex1 | 175 | 1213 | 4364 | 4278 | 14015 | 4904 | 17808 | 6876 | 31734 |
| PSA-013-int3′ | | #N/A | #N/A | #N/A | #N/A | #N/A | #N/A | 1093 | 1223 |
| PSA-014-int3′ | | 1956 | 1166 | 2420 | 2975 | #N/A | #N/A | 1539 | 3350 |
| PSA-015-ex1-ex2 | 176 | 955 | 2213 | 1408 | 2623 | 2483 | 4279 | 3154 | 6132 |
| PSA-015-ex1-ex2bis | 177 | 803 | 1250 | 1031 | 2020 | 1982 | 3736 | #N/A | #N/A |
| PSA-016-ex1-ex2 | 178 | #N/A | #N/A | 1075 | 1554 | 2189 | 1973 | 1314 | 1752 |
| PSA-018-jct-int2-int2 | 179 | 1842 | 868 | 3080 | 1807 | #N/A | #N/A | 1289 | 1570 |
| PSA-018-jct-ex1-ex2 | | 1164 | 1491 | 3238 | 3452 | 4262 | 4256 | 3139 | 3083 |
| PSA-018-int3′ | | #N/A | #N/A | #N/A | #N/A | 3336 | 1678 | #N/A | #N/A |
| PSA-019-ex3 | | 1609 | 867 | 7076 | 3002 | 6867 | 2373 | 8564 | 2978 |
| PSA-019-jct-ex4-5 | | 1509 | 1603 | 9695 | 7108 | 6934 | 5015 | 7853 | 6549 |
| PSA-019-jct-ex3 | 180 | #N/A | #N/A | 933 | 995 | #N/A | #N/A | #N/A | #N/A |
| PSA-020-jct-ex3 | | #N/A | #N/A | 1186 | 1576 | #N/A | #N/A | #N/A | #N/A |
| PSA-020-jxt-ex4-ex5 | | 1077 | 1030 | 3925 | 2953 | 4470 | 3366 | 5471 | 4014 |
| PSA-020-ex3 | | 2310 | 1699 | 7329 | 3559 | 10682 | 4571 | 15616 | 7008 |
| PSA-021-jct-ex3 | 181 | #N/A | #N/A | 1186 | 1576 | #N/A | #N/A | #N/A | #N/A |
| PSA-021-jct-ex3-2 | 182 | #N/A | #N/A | 894 | 975 | #N/A | #N/A | #N/A | #N/A |
| PSA-022-ex3 | | #N/A | #N/A | #N/A | #N/A | #N/A | #N/A | #N/A | #N/A |
| PSA-023-jct-ex2 | 183 | #N/A | #N/A | #N/A | #N/A | #N/A | #N/A | #N/A | #N/A |
| PSA-023-jct-ex5 | | 4620 | 1896 | [symbol] | [symbol] 92 | 20734 | 7239 | 26868 | 9448 |
| PSA-023-jct-in3-ex4 | 184 | 1444 | 1454 | 970 | 3835 | #N/A | #N/A | #N/A | #N/A |
| PSA-025-jct-ex2-ex4 | 185 | #N/A | #N/A | #N/A | #N/A | 4913 | 2868 | #N/A | #N/A |
| PSA-026-jct-ex3 | 186 | #N/A | #N/A | #N/A | #N/A | 2703 | 1801 | #N/A | #N/A |
| PSA-027-jct-ex3-ex5 | 187 | 1827 | 1596 | 2605 | 2260 | 2824 | 2702 | 4001 | 3913 |
| PSA-027-jct-ex3- | 188 | 1066 | 1430 | 1511 | 2501 | 2414 | 3802 | 2260 | 3981 |
| ex5bis | | | | | | | | | |
| PSA-001-jct-int1 | | #N/A | #N/A | 1658 | 4896 | 2802 | 8297 | 2736 | 7872 |
| PSA-d-jct-ex2-ex3 | 189 | 899 | 1198 | 2381 | 3067 | 3075 | 3513 | 3981 | 5169 |
| PSA-d-exon3 | | 4358 | 1731 | 19202 | 5982 | 14850 | 4686 | 22661 | 8002 |
| PSA-f-jct-int5′ | | #N/A | #N/A | #N/A | #N/A | #N/A | #N/A | #N/A | #N/A |
| PSA-f-jct-int2 | 190 | #N/A | #N/A | 963 | 1433 | 2810 | 1619 | #N/A | #N/A |
| PSA-g-jct-ex2-int2 | | 1028 | 1125 | 4946 | 2001 | #N/A | #N/A | #N/A | #N/A |
| PSA-g-jct-ex2-int2bis | | 1165 | 781 | #N/A | #N/A | #N/A | #N/A | #N/A | #N/A |
| PSA-h-jct-ex4-int4 | 191 | #N/A | #N/A | 973 | 1048 | #N/A | #N/A | #N/A | #N/A |
| PSA-h-jct3′ | | 941 | 739 | 2428 | 1270 | 2441 | 1288 | 2991 | 1551 |
| PSA-h-exon5bis | | 4717 | 1773 | 18338 | 7197 | 21552 | 8091 | 23252 | 9544 |
| PSA-j-jct-ex4-int4 | 192 | 1140 | 1063 | 5712 | 3235 | 3511 | 1443 | 5541 | 3161 |
| PSA-k-jct-ex4-int4 | 193 | #N/A | #N/A | 985 | 967 | #N/A | #N/A | #N/A | #N/A |
| PSA-k-jct-int4 | | 900 | 1365 | 1672 | 3283 | 2098 | 3747 | 2135 | 4933 |
| PSA-l-jct-ex4-int4 | 194 | 916 | 727 | 2584 | 1125 | 2846 | 1154 | 3852 | 1395 |
| PSA-l-jct-int3′ | | 1034 | 1107 | 1835 | 1436 | #N/A | #N/A | 1643 | 1859 |
| PSA-m-int1-5′ | | #N/A | #N/A | 1212 | 2706 | 2626 | 6088 | 1920 | 5380 |
| PSA-m-jct-int1 | 195 | #N/A | #N/A | #N/A | #N/A | 3216 | 1858 | #N/A | #N/A |
| PSA-n-int1-5′ | | #N/A | #N/A | 945 | 1174 | 1752 | 1284 | 1180 | 1923 |
| PSA-n-jct-int1 | 196 | 1519 | 868 | 3073 | 1209 | #N/A | #N/A | 3994 | 3765 |
| PSA-p-jct-ex1-int1 | 197 | 2449 | 1048 | #N/A | #N/A | #N/A | #N/A | #N/A | #N/A |
| PSA-q-jct-int5′ | | 3989 | 29327 | 13464 | 70837 | 14810 | 73656 | 20726 | 92817 |
| PSA-q-jct-int2 | 198 | #N/A | #N/A | #N/A | #N/A | 2067 | 1333 | 1514 | 1283 |
| PSA-r-jct-int2 | 199 | 3628 | 1631 | 1555 | 1144 | #N/A | #N/A | 2579 | 1504 |
| PSA-r-jct-int3′ | | #N/A | #N/A | [symbol] | #N/A | #N/A | #N/A | #N/A | #N/A |
| PSA-s-jct-int4 | 200 | 1189 | 914 | [symbol] | [symbol] 749 | #N/A | #N/A | 1440 | 2759 |
| PSA-s-jct-int3′ | | #N/A | #N/A | 864 | 1117 | #N/A | #N/A | 1340 | 1477 |
| PSA-t-jct-int5′ | | 1312 | 903 | 1764 | 1423 | #N/A | #N/A | 1421 | 1902 |
| PSA-t-jct-int4 | 201 | #N/A | #N/A | #N/A | #N/A | 2307 | 2412 | #N/A | #N/A |
| PSA-u-jct-int4-ex5 | 202 | #N/A | #N/A | 1617 | 2126 | 1747 | 2236 | 2340 | 3612 |
| KLK-2-exon1-wt | | 1208 | 1007 | 1687 | 1940 | 2300 | 2467 | 3081 | 3507 |
| KLK-2-exon2-wt | | 3102 | 1846 | 13579 | 6702 | 11306 | 6382 | 23052 | 12016 |
| KLK-2-exon2bis-wt | | 1023 | 780 | 1958 | 1291 | 2070 | 1365 | 2280 | 1695 |
| KLK-2-exon3-wt | | 2024 | 938 | 6616 | 1562 | 5854 | 1441 | 8572 | 2373 |
| KLK-2-exon4-wt | | 994 | 802 | 3740 | 2928 | 2444 | 1620 | 3301 | 2352 |
| KLK-2-exon5-wt | | 1907 | 1271 | 10323 | 4125 | 8435 | 4120 | 17038 | 8033 |
| KLK2-jctex1-2-wt | | #N/A | #N/A | 869 | 1152 | #N/A | #N/A | #N/A | #N/A |
| KLK2-jctex2-3-wt | | #N/A | #N/A | 1578 | 1095 | 2408 | 1313 | 2051 | 1155 |
| KLK2-jctex3-4-wt | | 2467 | 909 | 7977 | 1862 | 9261 | 2689 | 13732 | 3386 |
| KLK2-jctex4-5-wt | | 1049 | 1003 | 1888 | 1792 | 2483 | 1942 | 3326 | 2934 |
| KLK2-intron1-wt | | #N/A | #N/A | #N/A | #N/A | #N/A | #N/A | #N/A | #N/A |
| KLK2-intron2-wt | | #N/A | #N/A | 1013 | 2126 | 1608 | 2987 | #N/A | #N/A |
| KLK2-intron3-wt | | #N/A | #N/A | 1051 | 1004 | #N/A | #N/A | 1265 | 1362 |
| KLK2-intron4-wt | | 1501 | 925 | 1169 | 1378 | #N/A | #N/A | 1382 | 2993 |
| KLK2-jct-ex-int1wt | | #N/A | #N/A | #N/A | #N/A | #N/A | #N/A | #N/A | #N/A |
| KLK2-jct-ex-int2wt | | #N/A | #N/A | #N/A | #N/A | 1716 | 2095 | #N/A | #N/A |
| KLK2-jct-ex-int3wt | | #N/A | #N/A | #N/A | #N/A | #N/A | #N/A | #N/A | #N/A |
| KLK2-jct-ex-int4wt | | 3300 | 885 | 1776 | 1336 | #N/A | #N/A | #N/A | #N/A |
| KLK2-jct-ex-int6wt | | #N/A | #N/A | #N/A | #N/A | #N/A | #N/A | #N/A | #N/A |
| KLK2-jct-ex-int7wt | | #N/A | #N/A | 1186 | 1025 | #N/A | #N/A | 1560 | 1258 |
| KLK2-jct-ex-int8wt | | 832 | 900 | 1365 | 1265 | 1859 | 1291 | 2627 | 2202 |
| KLK2-002-jct-int2-ex3 | 203 | 2785 | 876 | 2131 | 1985 | 3769 | 1473 | #N/A | #N/A |
| KLK2-002-jct-ex3-ex4 | 204 | #N/A | #N/A | [symbol] | #N/A | #N/A | #N/A | #N/A | #N/A |
| KLK2-002-ex4 | | 1046 | 1106 | [symbol] | [symbol] 608 | 3232 | 3010 | 4571 | 4215 |
| KLK2-003-exon5 | | 3263 | 1622 | [symbol] | [symbol] 870 | 11784 | 4099 | 19228 | 8133 |
| KLK2-003-jct-ex1-ex3 | 205 | 1238 | 961 | 1945 | 1581 | 1768 | 1374 | 2228 | 2377 |
| KLK2-003-jct-int4-ex5 | 206 | #N/A | #N/A | 1023 | 1622 | 1492 | 1665 | 1352 | 2662 |
| KLK2-004-jct-ex2-ex4 | 207 | #N/A | #N/A | 8414 | 6017 | #N/A | #N/A | #N/A | #N/A |
| KLK2-004-int4 | | #N/A | #N/A | 1281 | 2549 | #N/A | #N/A | #N/A | #N/A |
| KLK2-005-int4 | | #N/A | #N/A | 1761 | 2260 | #N/A | #N/A | 2210 | 3978 |
| KLK2-008-ex4 | | 1296 | 906 | 2407 | 1407 | 2597 | 1464 | 1980 | 1377 |
| KLK2-008-jct-ex3-ex4 | | #N/A | #N/A | 889 | 962 | 2353 | 1090 | #N/A | #N/A |
| KLK2-009-jct-ex3-ex4 | 208 | #N/A | #N/A | 978 | 925 | #N/A | #N/A | 1189 | 1316 |
| KLK2-009-jct-ex3 | 209 | #N/A | #N/A | 3131 | 1397 | #N/A | #N/A | #N/A | #N/A |
| KLK2-009-ex4 | | #N/A | #N/A | #N/A | #N/A | #N/A | #N/A | #N/A | #N/A |
| KLK2-011-jct-ex3-ex4 | 210 | #N/A | #N/A | #N/A | #N/A | #N/A | #N/A | #N/A | #N/A |
| KLK2-011-jct-ex4-int4 | | 759 | 766 | 1324 | 1107 | 1622 | 1173 | 1739 | 1374 |
| KLK2-a-exon3 | | 1433 | 898 | 4830 | 1479 | 5675 | 1478 | 10361 | 2627 |
| KLK2-a-exon3bis | | 7899 | 2845 | 13296 | 2765 | 17883 | 4913 | 19143 | 5485 |
| KLK2-b-jct-int1 | 211 | #N/A | #N/A | #N/A | #N/A | #N/A | #N/A | #N/A | #N/A |
| KLK2-b-jct-int3′ | | #N/A | #N/A | 2806 | 2120 | 1840 | 1319 | 1239 | 2666 |
| KLK2-c-jct-int1 | 212 | #N/A | #N/A | 1934 | 1262 | #N/A | #N/A | #N/A | #N/A |
| KLK2-d-jct-int5′ | | #N/A | #N/A | #N/A | #N/A | #N/A | #N/A | #N/A | #N/A |
| KLK2-d-jct-int1 | 213 | 1147 | 1370 | 948 | 2135 | #N/A | #N/A | #N/A | #N/A |
| KLK2-e-ex2 | | 844 | 843 | 1709 | 1516 | 2332 | 1838 | 2333 | 1720 |
| KLK2-e-ex2-140nt? | 214 | 3609 | 1344 | 2066 | 1262 | #N/A | #N/A | 1439 | 2081 |
| KLK2-e-jct-140nt?- | 215 | #N/A | #N/A | #N/A | #N/A | #N/A | #N/A | #N/A | #N/A |
| ex3 | | | | | | | | | |
| KLK2-f-jct-ex2-int2 | 216 | #N/A | #N/A | #N/A | #N/A | #N/A | #N/A | 3710 | 2735 |
| KLK2-f-jct3′ | | #N/A | #N/A | 2013 | 2496 | #N/A | #N/A | 1465 | 2138 |
| KLK2-g-ex5 | | 2661 | 2345 | 11404 | 5898 | 10871 | 6663 | 25958 | 14694 |
| KLK2-g-int4 | | 980 | 741 | 1535 | 1156 | 1920 | 1148 | 2033 | 1422 |
| KLK2-h-jct3′ | | 1352 | 969 | 4132 | 1806 | 4947 | 2295 | 5766 | 2551 |
| KLK2-h-jct3′bis | | 1726 | 858 | [symbol] | [symbol] 14 | 7094 | 1922 | 8441 | 2234 |
| KLK2-i-ex4 | | #N/A | #N/A | [symbol] | [symbol] 48 | 2575 | 1397 | 4752 | 2614 |
| KLK2-j-jct-int3′ | | #N/A | #N/A | [symbol] | [symbol] 52 | #N/A | #N/A | #N/A | #N/A |
| KLK2-j-jct-int2 | 217 | 1914 | 1847 | 1048 | 2856 | 1631 | 4416 | 1424 | 4402 |
| KLK2-k-jct-int5′ | 218 | #N/A | #N/A | 1297 | 3250 | 1809 | 4193 | 2320 | 6264 |
| KLK2-k-jct-int5′-6nt- | 219 | 1373 | 1350 | 3168 | 2224 | 4908 | 3072 | 8481 | 5989 |
| ex5 | | | | | | | | | |
| KLK2-l-jct-int2-ex3 | 220 | #N/A | #N/A | #N/A | #N/A | #N/A | #N/A | #N/A | #N/A |

TABLE 5

| | SEQ | patient15068 | | patient9648 | | patient8827 | | patient10063 | |
|---|---|---|---|---|---|---|---|---|---|
| | ID | LEX.benin - | LEX.Tumor | LEX.benin - | LEX.Tumor | LEX.benin - | LEX.Tumor | LEX.benign - | LEX.Tumor |
| Oligonucleotide | NO | BG | Norm. | BG | Norm. | BG | Norm. | BG | Norm. |
| PSA-exon1-wt | | 4768 | 6273 | 3826 | 5600 | 2601 | 3458 | 10451 | 9383 |
| PSA-exon2-wt | | 5779 | 7258 | 2278 | 5641 | 1104 | 2267 | 10212 | 10367 |
| PSA-exon2bis-wt | | 2462 | 3383 | 1296 | 2122 | 807 | 961 | 4392 | 5049 |
| PSA-exon3-wt | | 14805 | 17594 | 4881 | 12028 | 4606 | 10985 | 15573 | 14987 |
| PSA-exon4-wt | | 7297 | 6707 | 2817 | 4635 | 1446 | 3054 | 12933 | 9720 |
| PSA-exon5-wt | | 34320 | 26749 | 7839 | 15212 | 2364 | 5386 | 34033 | 27809 |
| PSA-jctex1-2-wt | | #N/A | #N/A | #N/A | #N/A | #N/A | #N/A | #N/A | #N/A |
| PSA-jctex2-3-wt | | 3245 | 3928 | 1106 | 2456 | 1209 | 2049 | 3145 | 2529 |
| PSA-jctex3-4-wt | | 15637 | 16190 | 5606 | 13648 | 2961 | 7663 | 18472 | 14904 |
| PSA-jctex4-5-wt | | 13083 | 12337 | 3532 | 6998 | 1549 | 3640 | 10098 | 8699 |
| PSA-jct-ex-int1wt | | #N/A | #N/A | #N/A | #N/A | #N/A | #N/A | #N/A | #N/A |
| PSA-jct-ex-int2wt | | 1293 | 1758 | #N/A | #N/A | 1047 | 1077 | #N/A | #N/A |
| PSA-jct-ex-int3wt | | 1137 | 1290 | 1614 | 1458 | 1914 | 1944 | #N/A | #N/A |
| PSA-jct-ex-int4wt | | #N/A | #N/A | 793 | 1022 | #N/A | #N/A | 562 | 1142 |
| PSA-jct-ex-int5wt | | #N/A | #N/A | #N/A | #N/A | #N/A | #N/A | #N/A | #N/A |
| PSA-jct-ex-int6wt | | 1353 | 1368 | #N/A | #N/A | #N/A | #N/A | 1815 | 2154 |
| PSA-jct-ex-int7wt | | 3232 | 3485 | 1916 | 3544 | 1128 | 2706 | #N/A | #N/A |
| PSA-jct-ex-int8wt | | 2336 | 2241 | 1005 | 1244 | #N/A | #N/A | 2289 | 2175 |
| PSA-intron 1 | | 2093 | 3008 | 1790 | 2578 | 1314 | 1329 | 1254 | 1620 |
| PSA-intron 2 | | #N/A | #N/A | #N/A | #N/A | #N/A | #N/A | #N/A | #N/A |
| PSA-intron 2bis | | #N/A | #N/A | #N/A | #N/A | #N/A | #N/A | #N/A | #N/A |
| PSA-intron 4 | | #N/A | #N/A | #N/A | #N/A | #N/A | #N/A | #N/A | #N/A |
| PSA-001-int-int | 168 | 1101 | 1565 | 957 | 1185 | 917 | 806 | #N/A | #N/A |
| PSA-001-int-3′ | | 2606 | 2964 | 3185 | 4599 | 1066 | 2151 | 1140 | 1709 |
| PSA-001-int3′bis | | 2406 | 2510 | 1525 | 1969 | 824 | 2140 | 1611 | 1254 |
| PSA-003-int-int | 169 | 7299 | 9980 | 8214 | 12116 | 4605 | 5931 | 5547 | 8579 |
| PSA-003-int3′ | | 1448 | 1584 | #N/A | #N/A | #N/A | #N/A | #N/A | #N/A |
| PSA-004-jctex1-int | 170 | #N/A | #N/A | #N/A | #N/A | #N/A | #N/A | #N/A | #N/A |
| PSA-004intron1 | | 2485 | 2732 | 1858 | 1980 | 1184 | 1694 | 1776 | 1863 |
| PSA-005-jct-int1-int1 | 171 | 1969 | 2501 | 1674 | 2629 | 1102 | 1342 | 860 | 1118 |
| PSA-008-jctex1-int | 172 | #N/A | #N/A | #N/A | #N/A | #N/A | #N/A | #N/A | #N/A |
| PSA-009-jct-int2-int2 | 173 | 2703 | 3739 | 3540 | 7540 | 1972 | 2622 | 2346 | 4551 |
| PSA-009-int3′ | | #N/A | #N/A | #N/A | #N/A | #N/A | #N/A | #N/A | #N/A |
| PSA-009-int3′bis | | #N/A | #N/A | #N/A | #N/A | #N/A | #N/A | #N/A | #N/A |
| PSA-010-jxt-ex1-ex2 | | #N/A | #N/A | #N/A | #N/A | #N/A | #N/A | 1448 | 1392 |
| PSA-0012-jctex2-int2 | 174 | #N/A | #N/A | #N/A | #N/A | #N/A | #N/A | #N/A | #N/A |
| PSA-012-int3′ | | 5033 | 6197 | 4007 | 5195 | 2817 | 3384 | 5965 | 6450 |
| PSA-013-jct-int1-ex1 | 175 | 5955 | 9357 | 4274 | 6547 | 2891 | 4815 | 5663 | 6240 |
| PSA-013-int3′ | | #N/A | #N/A | #N/A | #N/A | #N/A | #N/A | #N/A | #N/A |
| PSA-014-int3′ | | #N/A | #N/A | #N/A | #N/A | 1284 | 1086 | #N/A | #N/A |
| PSA-015-ex1-ex2 | 176 | 1153 | 1851 | 1165 | 1220 | 729 | 752 | 2051 | 1840 |
| PSA-015-ex1-ex2bis | 177 | #N/A | #N/A | 689 | 935 | #N/A | #N/A | #N/A | #N/A |
| PSA-016-ex1-ex2 | 178 | #N/A | #N/A | #N/A | #N/A | #N/A | #N/A | 553 | 3140 |
| PSA-018-jct-int2-int2 | 179 | #N/A | #N/A | #N/A | #N/A | #N/A | #N/A | 482 | 3913 |
| PSA-018-jct-ex1-ex2 | | #N/A | #N/A | #N/A | #N/A | #N/A | #N/A | #N/A | #N/A |
| PSA-018-int3′ | | #N/A | #N/A | #N/A | #N/A | #N/A | #N/A | #N/A | #N/A |
| PSA-019-ex3 | | 3930 | 5179 | 911 | 2385 | 736 | 1375 | 4566 | 4306 |
| PSA-019-jct-ex4-5 | | 7053 | 6396 | 2245 | 4645 | 2078 | 3915 | 11208 | 11231 |
| PSA-019-jct-ex3 | 180 | 2291 | 3229 | #N/A | #N/A | #N/A | #N/A | 1217 | 1471 |
| PSA-020-jct-ex3 | | #N/A | #N/A | #N/A | #N/A | #N/A | #N/A | #N/A | #N/A |
| PSA-020-jxt-ex4-ex5 | | 3801 | 3806 | 984 | 2127 | #N/A | #N/A | 3946 | 3640 |
| PSA-020-ex3 | | 8961 | 10722 | 3077 | 7482 | 2526 | 5790 | 7596 | 6781 |
| PSA-021-jct-ex3 | 181 | #N/A | #N/A | #N/A | #N/A | #N/A | #N/A | #N/A | #N/A |
| PSA-021-jct-ex3-2 | 182 | #N/A | #N/A | #N/A | #N/A | #N/A | #N/A | #N/A | #N/A |
| PSA-022-ex3 | | #N/A | #N/A | #N/A | #N/A | #N/A | #N/A | #N/A | #N/A |
| PSA-023-jct-ex2 | 183 | #N/A | #N/A | #N/A | #N/A | #N/A | #N/A | 754 | 1084 |
| PSA-023-jct-ex5 | | 29235 | 21359 | 6201 | 11358 | 4424 | 8194 | 15565 | 15432 |
| PSA-023-jct-in3-ex4 | 184 | #N/A | #N/A | #N/A | #N/A | #N/A | #N/A | #N/A | #N/A |
| PSA-025-jct-ex2-ex4 | 185 | #N/A | #N/A | #N/A | #N/A | #N/A | #N/A | #N/A | #N/A |
| PSA-026-jct-ex3 | 186 | #N/A | #N/A | #N/A | #N/A | #N/A | #N/A | #N/A | #N/A |
| PSA-027-jct-ex3-ex5 | 187 | 1062 | 1406 | #N/A | #N/A | #N/A | #N/A | 2158 | 1916 |
| PSA-027-jct-ex3-ex5bis | 188 | 1637 | 1688 | 1914 | 2855 | 838 | 910 | #N/A | #N/A |
| PSA-001-jct-int1 | | 2950 | 3775 | 2478 | 3653 | 1836 | 1948 | 1200 | 1797 |
| PSA-d-jct-ex2-ex3 | 189 | 3281 | 3736 | 1504 | 2473 | 901 | 1337 | 1576 | 1486 |
| PSA-d-exon3 | | 11672 | 11510 | 3719 | 8979 | 2936 | 5949 | 7376 | 5820 |
| PSA-f-jct-int5′ | | #N/A | #N/A | 1089 | 1866 | 707 | 2180 | #N/A | #N/A |
| PSA-f-jct-int2 | 190 | 1060 | 1300 | 737 | 930 | #N/A | #N/A | #N/A | #N/A |
| PSA-g-jct-ex2-int2 | | #N/A | #N/A | #N/A | #N/A | #N/A | #N/A | #N/A | #N/A |
| PSA-g-jct-ex2-int2bis | | #N/A | #N/A | #N/A | #N/A | #N/A | #N/A | #N/A | #N/A |
| PSA-h-jct-ex4-int4 | 191 | #N/A | #N/A | #N/A | #N/A | #N/A | #N/A | #N/A | #N/A |
| PSA-h-jct3′ | | 2374 | 2325 | 761 | 1241 | #N/A | #N/A | 1567 | 1403 |
| PSA-h-exon5bis | | 27696 | 22674 | 4796 | 10122 | 2877 | 6389 | 21565 | 21790 |
| PSA-j-jct-ex4-int4 | 192 | 4464 | 4488 | 2068 | 4240 | 1643 | 3835 | 1882 | 2072 |
| PSA-k-jct-ex4-int4 | 193 | #N/A | #N/A | #N/A | #N/A | #N/A | #N/A | #N/A | #N/A |
| PSA-k-jct-int4 | | 2255 | 2526 | 1601 | 2084 | 814 | 1165 | 1066 | 1093 |
| PSA-l-jct-ex4-int4 | 194 | 2985 | 3043 | 1146 | 2238 | #N/A | #N/A | 1238 | 1964 |
| PSA-l-jct-int3′ | | #N/A | #N/A | #N/A | #N/A | 1512 | 916 | #N/A | #N/A |
| PSA-m-int1-5′ | | 2643 | 3101 | 2228 | 3216 | 1649 | 2071 | 5836 | 7488 |
| PSA-m-jct-int1 | 195 | #N/A | #N/A | #N/A | #N/A | #N/A | #N/A | #N/A | #N/A |
| PSA-n-int1-5′ | | 3017 | 3029 | 722 | 1541 | #N/A | #N/A | #N/A | #N/A |
| PSA-n-jct-int1 | 196 | #N/A | #N/A | #N/A | #N/A | #N/A | #N/A | #N/A | #N/A |
| PSA-p-jct-ex1-int1 | 197 | #N/A | #N/A | #N/A | #N/A | #N/A | #N/A | #N/A | #N/A |
| PSA-q-jct-int5′ | | 31152 | 39836 | 23996 | 32879 | 17391 | 13278 | 25338 | 18161 |
| PSA-q-jct-int2 | 198 | #N/A | #N/A | [symbol] | [symbol] | #N/A | #N/A | #N/A | #N/A |
| PSA-r-jct-int2 | 199 | #N/A | #N/A | #N/A | #N/A | #N/A | #N/A | #N/A | #N/A |
| PSA-r-jct-int3′ | | #N/A | #N/A | #N/A | #N/A | #N/A | #N/A | #N/A | #N/A |
| PSA-s-jct-int4 | 200 | #N/A | #N/A | 1094 | 930 | #N/A | #N/A | #N/A | #N/A |
| PSA-s-jct-int3′ | | #N/A | #N/A | 667 | 1119 | #N/A | #N/A | #N/A | #N/A |
| PSA-t-jct-int5′ | | #N/A | #N/A | #N/A | #N/A | #N/A | #N/A | #N/A | #N/A |
| PSA-t-jct-int4 | 201 | #N/A | #N/A | #N/A | #N/A | #N/A | #N/A | #N/A | #N/A |
| PSA-u-jct-int4-ex5 | 202 | 2179 | 1882 | 815 | 1065 | #N/A | #N/A | #N/A | #N/A |
| KLK-2-exon1-wt | | 1861 | 2437 | 828 | 1364 | #N/A | #N/A | 1446 | 1454 |
| KLK-2-exon2-wt | | 8270 | 9547 | 2838 | 6743 | 2317 | 5476 | 6196 | 6621 |
| KLK-2-exon2bis-wt | | 2195 | 2785 | 955 | 1499 | #N/A | #N/A | 3263 | 3312 |
| KLK-2-exon3-wt | | 3983 | 4106 | 1267 | 2294 | #N/A | #N/A | 3322 | 2543 |
| KLK-2-exon4-wt | | 3179 | 2612 | 741 | 1286 | 6013 | 5298 | 3254 | 2845 |
| KLK-2-exon5-wt | | 8872 | 6800 | 1838 | 2984 | 1982 | 3589 | 5857 | 5979 |
| KLK2-jctex1-2-wt | | #N/A | #N/A | #N/A | #N/A | #N/A | #N/A | 541 | 5408 |
| KLK2-jctex2-3-wt | | #N/A | #N/A | #N/A | #N/A | #N/A | #N/A | #N/A | #N/A |
| KLK2-jctex3-4-wt | | 11355 | 11376 | 2546 | 5873 | 996 | 2171 | 7370 | 5776 |
| KLK2-jctex4-5-wt | | 6355 | 4585 | 2044 | 3592 | 1014 | 1652 | 4262 | 4167 |
| KLK2-intron1-wt | | 1238 | 2322 | #N/A | #N/A | #N/A | #N/A | #N/A | #N/A |
| KLK2-intron2-wt | | #N/A | #N/A | #N/A | #N/A | #N/A | #N/A | #N/A | #N/A |
| KLK2-intron3-wt | | #N/A | #N/A | #N/A | #N/A | #N/A | #N/A | #N/A | #N/A |
| KLK2-intron4-wt | | #N/A | #N/A | #N/A | #N/A | #N/A | #N/A | #N/A | #N/A |
| KLK2-jct-ex-int1wt | | #N/A | #N/A | #N/A | #N/A | 1235 | 1136 | #N/A | #N/A |
| KLK2-jct-ex-int2wt | | #N/A | #N/A | #N/A | #N/A | #N/A | #N/A | #N/A | #N/A |
| KLK2-jct-ex-int3wt | | #N/A | #N/A | #N/A | #N/A | #N/A | #N/A | #N/A | #N/A |
| KLK2-jct-ex-int4wt | | #N/A | #N/A | #N/A | #N/A | #N/A | #N/A | #N/A | #N/A |
| KLK2-jct-ex-int6wt | | #N/A | #N/A | #N/A | #N/A | #N/A | #N/A | #N/A | #N/A |
| KLK2-jct-ex-int7wt | | 1453 | 1441 | #N/A | #N/A | #N/A | #N/A | 968 | 956 |
| KLK2-jct-ex-int8wt | | 3273 | 3241 | [symbol] | [symbol] | 739 | 827 | 1896 | 1893 |
| KLK2-002-jct-int2-ex3 | 203 | #N/A | #N/A | [symbol] | [symbol] | 4290 | 17851 | #N/A | #N/A |
| KLK2-002-jct-ex3-ex4 | 204 | #N/A | #N/A | #N/A | #N/A | #N/A | #N/A | #N/A | #N/A |
| KLK2-002-ex4 | | 4957 | 5034 | 1849 | 3506 | 858 | 1317 | 2874 | 2736 |
| KLK2-003-exon5 | | 22655 | 18828 | 3405 | 6002 | 1056 | 2377 | 10645 | 9294 |
| KLK2-003-jct-ex1-ex3 | 205 | #N/A | #N/A | #N/A | #N/A | 907 | 757 | 448 | 1913 |
| KLK2-003-jct-int4-ex5 | 206 | #N/A | #N/A | #N/A | #N/A | #N/A | #N/A | #N/A | #N/A |
| KLK2-004-jct-ex2-ex4 | 207 | #N/A | #N/A | #N/A | #N/A | #N/A | #N/A | #N/A | #N/A |
| KLK2-004-int4 | | #N/A | #N/A | #N/A | #N/A | #N/A | #N/A | #N/A | #N/A |
| KLK2-005-int4 | | #N/A | #N/A | #N/A | #N/A | #N/A | #N/A | 551 | 3065 |
| KLK2-008-ex4 | | 5448 | 4354 | 2593 | 3290 | 1079 | 1557 | 3778 | 3253 |
| KLK2-008-jct-ex3-ex4 | | #N/A | #N/A | 1804 | 1416 | 1056 | 1081 | #N/A | #N/A |
| KLK2-009-jct-ex3-ex4 | 208 | 1016 | 1849 | #N/A | #N/A | #N/A | #N/A | #N/A | #N/A |
| KLK2-009-jct-ex3 | 209 | #N/A | #N/A | #N/A | #N/A | #N/A | #N/A | 420 | 4034 |
| KLK2-009-ex4 | | 6561 | 6779 | 3494 | 5753 | 2088 | 2806 | 10429 | 8752 |
| KLK2-011-jct-ex3-ex4 | 210 | 1883 | 4970 | 864 | 1915 | #N/A | #N/A | #N/A | #N/A |
| KLK2-011-jct-ex4-int4 | | 2072 | 1988 | 681 | 1071 | 1138 | 802 | 1490 | 1520 |
| KLK2-a-exon3 | | 7550 | 8300 | 1727 | 3408 | 1109 | 1854 | 5606 | 4894 |
| KLK2-a-exon3bis | | 12547 | 13954 | 4272 | 10033 | 2134 | 5350 | 12484 | 10192 |
| KLK2-b-jct-int1 | 211 | #N/A | #N/A | #N/A | #N/A-{}- | #N/A | #N/A | 543 | 974 |
| KLK2-b-jct-int3′ | | 1048 | 1450 | #N/A | #N/A | #N/A | #N/A | #N/A | #N/A |
| KLK2-c-jct-int1 | 212 | #N/A | #N/A | #N/A | #N/A | #N/A | #N/A | #N/A | #N/A |
| KLK2-d-jct-int5′ | | #N/A | #N/A | #N/A | #N/A | #N/A | #N/A | #N/A | #N/A |
| KLK2-d-jct-int1 | 213 | 1220 | 1971 | 1075 | 1462 | 654 | 752 | 534 | 895 |
| KLK2-e-ex2 | | 2903 | 3915 | 1033 | 1872 | 1356 | 2054 | 1997 | 1649 |
| KLK2-e-ex2-140nt? | 214 | #N/A | #N/A | 1313 | 33056 | 1312 | 4078 | #N/A | #N/A |
| KLK2-e-jct-140nt?-ex3 | 215 | #N/A | #N/A | #N/A | #N/A | #N/A | #N/A | #N/A | #N/A |
| KLK2-f-jct-ex2-int2 | 216 | #N/A | #N/A | #N/A | #N/A | #N/A | #N/A | #N/A | #N/A |
| KLK2-f-jct3′ | | #N/A | #N/A | #N/A | #N/A | #N/A | #N/A | #N/A | #N/A |
| KLK2-g-ex5 | | 19156 | 13670 | 3516 | 6837 | 1513 | 2972 | 10239 | 10753 |
| KLK2-g-int4 | | 2577 | 2656 | [symbol] | [symbol] | #N/A | #N/A | 2216 | 2696 |
| KLK2-h-jct3′ | | 4731 | 4807 | [symbol] | [symbol] | #N/A | #N/A | 4442 | 3777 |
| KLK2-h-jct3′bis | | 9863 | 7729 | [symbol] | [symbol] | 858 | 1466 | 5392 | 4410 |
| KLK2-i-ex4 | | 5300 | 3761 | 1543 | 2050 | 840 | 1135 | 3321 | 3378 |
| KLK2-j-jct-int3′ | | #N/A | #N/A | #N/A | #N/A | #N/A | #N/A | #N/A | #N/A |
| KLK2-j-jct-int2 | 217 | 1593 | 2397 | 1197 | 1746 | 772 | 928 | #N/A | #N/A |
| KLK2-k-jct-int5′ | 218 | 2326 | 3311 | 1532 | 2403 | 874 | 1102 | 1032 | 1465 |
| KLK2-k-jct-int5′-6nt-ex5 | 219 | 8177 | 7327 | 2056 | 4363 | 1320 | 2722 | 4063 | 4249 |
| KLK2-l-jct-int2-ex3 | 220 | #N/A | #N/A | #N/A | #N/A | #N/A | #N/A | #N/A | #N/A |

1-24. (canceled)

25. A nucleic acid comprising a sequence chosen from among:
a) sequences SEQ ID NO: 1 to 49,
b) a variant of sequences SEQ ID NO: 1 to 49 resulting from the degeneracy of the genetic code,
c) the complementary strand of sequences SEQ ID NO: 1 to 49, and
d) a specific fragment of sequences a) to c).

26. A nucleic acid of claim 25, wherein the nucleic acid is DNA or RNA.

27. A polypeptide encoded by a nucleic acid of claim 25.

28. A polypeptide of claim 27, chosen from a polypeptide comprising all or a specific part of a sequence chosen from SEQ ID Nos: 50 to 167.

29. A polypeptide of claim 27, wherein said polypeptide is a protein chosen from variants KLK2-EHT002 to KLK2-EHT011 and PSA-EHT001 to PSA-EHT027 or KLK2-EHTb to KLK2-EHTl and PSA-EHTa to PSA-EHTu of sequence SEQ ID Nos: 50 to 167, respectively.

30. A nucleic acid probe wherein the probe allows the detection by selective hybridisation of a nucleic acid of claim 25.

31. Probe of claim 30, wherein the probe comprises a sequence of said nucleic acid.

32. Probe according to claim 31, wherein the probe comprises from 20 to 1000 nucleotides, preferably from 50 to 800.

33. A primer, wherein the primer allows the selective amplification of a nucleic acid of claim 25.

34. A primer according to claim 33, wherein the primer is composed of 3 to 50 bases.

35. A primer of claim 33, wherein the primer is complementary to at least one region of the gene encoding the specific antigen of PSA, or of that encoding KLK2, containing a mutation involved in a cancer.

36. A primer according to claim 35, wherein the primer is composed of a single-stranded nucleic acid comprising from 3 to 50 nucleotides complementary to at least part of a sequence selected from SEQ ID NO: 1 to 49 or their complementary strand.

37. A primer pair comprising a sense sequence and a reverse sequence, wherein the primers of said pair hybridise to a region of a nucleic acid according to claim 25 and allow amplification of at least a portion of said nucleic acid.

38. An antibody, wherein the antibody is specific for a protein or a polypeptide of claim 28.

39. An antibody of claim 38, wherein the antibody is polyclonal, monoclonal or a derivative thereof.

40. A method for detecting a disease or predisposition to a disease in a subject, comprising determining the presence, in a sample from said subject, of a nucleic acid of claim 25 or of a polypeptide encoded by said nucleic acid.

41. The method of claim 40, wherein the determination is performed by sequencing, selective hybridisation or amplification.

42. A method of claim 41, wherein the amplification is performed by using a primer pair comprising a sense sequence and a reverse sequence wherein the primers of said pair hybridize to a region of said nucleic acid and allow amplification of at least a portion of said nucleic acid.

43. A kit comprising
i. a primer pair of claim 37 or a probe which allows the detection by selective hybridization to said nucleic acid or an antibody specific for a polypeptide comprising all or a specific part of a sequence selected from SEQ ID NOS. 50 to 167, and
ii. the reagents necessary for an amplification, a hybridisation or an immunological reaction.

44. A method for selecting or identifying active compounds, comprising contacting a test compound in vitro or ex vivo with a cell expressing a polypeptide comprising a sequence as defined in claim 27, and selecting or identifying compounds that modulate the expression or activity of said polypeptide.

45. A method of claim 44, wherein the method comprises selecting compounds that bind to said polypeptide.

46. A method of claim 44, wherein the method comprises selecting compounds that modulate the expression of said polypeptide.

47. A vector containing a nucleic acid of claim 25.

48. A recombinant cell containing a vector of claim 47.

49. A product comprising a nucleic acid of claim 25, a vector containing said nucleic acid, a polypeptide encoded by said nucleic acid or an antibody specific for a polypeptide comprising all or a specific part of a sequence selected from SEQ ID NOS. 50 to 167 immobilised on a matrix.


SEQUENCE LISTING (220 sequences)

SEQ ID NO: 1 - DNA, 550 bp, Homo sapiens
cctgtgtcagcatgtgggacctggttctctccatcgccttgtctgtggggtgcactggtgccgtgcccctcatccagtctcggattgtgggaggctgggagtgcgagaagcattcccaaccctggcaggtggctgtgtacagtcatggatgggcacactgtgggggtgtcctggtgcacccccagtgggtgctcacagctgcccattgcctaaagaagtaagtaggaccctgggatctggggagggaatggctgtgtcccacaggaataacagcgggatgcttcccccagggtcacttctcaggaatagccaggtctggctgggtcggcacaacctgtttgagcctgaagacacaggccaaagggtccctgtcagccacagcttcccacacccgctctacaatatgagccttctgaagcatcaaagccttagaccagatgaagactccagccatgacctcatgctgcttcgcctgtcagagcctgccaagatcacagatgttgtgaaggagtcttcagtgtgtgagcctccatctcctgtccaatgacat

SEQ ID NO: 2 - DNA, 688 bp, Homo sapiens
cctgtgtcagcatgtgggacctggttctctccatcgccttgtctgtggggtgcactggaatagccaggtctggctgggtcgccacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgccacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaagggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtccca

SEQ ID NO: 3 - DNA, 436 bp, Homo sapiens
cctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaatcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaaagtgagtgaagaccctaattctgggctgcaatctgaaagctaacca

SEQ ID NO: 4 - DNA, 476 bp, Homo sapiens
cctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgcttcgcctgtcagagcctgccaagatcacagatgttgtgaaggagtcttcagtgtgtgagcctccatctcctgtccaatgacatgtgtgctagagc

SEQ ID NO: 5 - DNA, 723 bp, Homo sapiens
cctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtggggtgagtcatccctactcccaacatctggaggggaaaggtgagtgaagaccctaattctgggctgcaatctgaaagctaacca

SEQ ID NO: 6 - DNA, 409 bp, Homo sapiens
cctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacagccgctctacaatatgagccttctgaagcatcaaagccttagaccagatgaagactccagcatcgaaccagaggagtgagtcttcagtgtgtgagcctccatctc

SEQ ID NO: 7 - DNA, 595 bp, Homo sapiens
gcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagtggtaaagacacttgtggggtgagtcatccctactcccaacatctggaggggaaaggtgagtgaagaccctaattctgggctgcaatctgaaagctaacca

SEQ ID NO: 8 - DNA, 350 bp, Homo sapiens
ggttctctccatcgccttgtctgtggggtgcactggtgagattggggggataaaggaaggggggcgggttctgactcttatgctgaagcccttttcgtcccacccagtgccccagcctcgtcccttcagcccacagttcagcccagacaatgtgcccctgactcttccccccgaggctatctggcctgagacaacaaatgctgcctcccaccccgagtctggcactgggactttcagaactcctcctttcctgactctttgccccagacccgtcattcaatggctagctttttccatgggaaaaacacgagcacccccaaccacaacggccagttctctgattccctaaa

SEQ ID NO: 9 - DNA, 118 bp, Homo sapiens
ggttctctccatcgccttgtctgtggggtgcactgacccgtcattcaatggctagctttttccatgggaaaaacacgagcacccccaaccacaacggccagttctctgattccctaaa

SEQ ID NO: 10 - DNA, 159 bp, Homo sapiens
ggttctctccatcgccttgtctgtggggtgcactggtgagattggggggataaaggaaggggggcgggttctgactcttatgctgaagcccttttcctcccacccagtgccccagcctcgtccccccaaccacaacggccagttctctgattccctaaa

SEQ ID NO: 11 - DNA, 293 bp, Homo sapiens
cagtcatggatgggcacactgtgggggtgctttggagcccccccagcggggccgtacacaccatgcaccctggccgtaccgaaactagtggaaccctgctatctgccgagctagaggcatagctcgactattctatagtggcaactatacccccaggtttttttctcaggaatagccaggtctggctgggtcggcacaacctgtttgagcctgaagacacaggccagagggtccctgtcagccacagcttcccacacccgctctacaatatgagccttctgaagcatcaaagc

SEQ ID NO: 12 - DNA, 313 bp, Homo sapiens
cagtcatggatgggcacactcctgttttctaactcactgtctgtatttcaccacgactatatctccccgacccctgtgcttttctcactgtttctttttcttccctttggagtctcccttatcctcccctgccccatctacctttccccattttctctctcctcatgcatccacccccttcctccccaggaatagccaggtctggctgggtcggcacaacctgtttgagcctgaagacacaggccagagggtccctgtcagccacagcctcccacacccgctctacaatatgagccttctgaagcatcaaagc

SEQ ID NO: 13 - DNA, 211 bp, Homo sapiens
agctcaatgtgtgtgcatgtgagggggtgcctgtcattgcacagcactctctgcaggacatccctccaccctcatgcctctcctcctccccctgctactccacactcctcagatgcccccgtggcctccctcctttttctctcccacactgtatcacccctggcttcctctctgctgtttctccttctctctctgacttcccgcatccttt

SEQ ID NO: 14 - DNA, 216 bp, Homo sapiens
ggagtgacgatgaggatgacctgggggtggctccaggcattgtccccacctgggccctcacccagcctccctcacaggctcctggccctcagtctctcccctccactccattctccacctacccacagtgggtcattctgatcactgaactgaccatgccagccctgccgatggcccacttgtctgtaatggggtgcttcaaggtatcacatcatg

SEQ ID NO: 15 - DNA, 159 bp, Homo sapiens
ccactacagagccctcactccagccccagctgcaggaatagccaggtctggctgggtcggcacaacctgtttgagcctgaagacacaggccagagggtccctgtcagccacagcttcccacacccgctctacaatatgagccttctgaagcatcaaagc

SEQ ID NO: 16 - DNA, 455 bp, Homo sapiens
ggagagctgtgtcaccatgtgggtcccggttgtcttcctcaccctgtccgtgacgtggattggtgagaggggccatggtgggggggatgcaggagagggagccatccctgactgtcaagctgaggctctttgccccccaacccagcaccccagctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtgagtgtccaaccctatcccagagaccttgatgcttggcctcccaatcttgccctaggatacccagatgccaaccagacacctccttcttcctagccaggctatctggcctgagacaacaaatgggtc

SEQ ID NO: 17 - DNA, 444 bp, Homo sapiens
ggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccactccaagcccatacccccagcccctcccacttaccccagaactttctccccattgcccagccagctccctgctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtga

SEQ ID NO: 18 - DNA, 193 bp, Homo sapiens
ggagagctgtgtcaccatgtgggtcccggttgtcttcctcaccctgtccgtgacgtggattgctgtgagtgtccaaccctatcccagagaccttgatgcttggcctcccaatcttgccctaggatacccagatgccaaccagacacctccttcttcctagccaggctatctggcctgagacaacaaatgggtc

SEQ ID NO: 19 - DNA, 256 bp, Homo sapiens
ggagagctgtgtcaccatgtgggtcccggttgtcttcctcaccctgtccgtgacgtggattggtgagaggggccatggttggggggatgcaggagagggagccagccctgactgtcaagctgaggctctttccccccaaccctatcccagagaccttgatgcttggcctcccaatcttgccctaggatacccagatgccaaccagacacctccttcttcctagccaggctatctggcctgagacaacaaatgggtc

SEQ ID NO: 20 - DNA, 159 bp, Homo sapiens
ggagagctgtgtcaccatgtgggtcccggttgtcttccccaaccctatcccagagaccttgatgcttggcctcccaatcttgccctaggatacccagatgccaaccagacacctccttcttcctagccaggctatctggcctgagacaacaaatgggtc

SEQ ID NO: 21 - DNA, 133 bp, Homo sapiens
ggagagctgtgtcaccatgttggtcccggttgtcttcctcaccctgtccgtgacgtggattggatacccagatgccaaccagacacctccttcttcctagccaggctatctggcctgagacaacaaatgggtc

SEQ ID NO: 22 - DNA, 767 bp, Homo sapiens
ggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagagaaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtcttttcgtgggactattatgttgttttttttcctctcttctctgtcttcagtatccatatctccgcctcttattccattctttcttcaggacatccctcctctcttgggaggcgcagagaaggagtggttccagctggagctgggaggggcaattgagggaggaggaaggagaagggggaaggaaaacagggtatgggggaaaggaccctggggagggaagtggaggatgcatccttgggcctgcaggccaggctgcctacccacttggaaacccacgccaaagccgca

SEQ ID NO: 23 - DNA, 394 bp, Homo sapiens
ggagatgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaatctccatatccccccctctctctgtccttctctagtccctctctagccagtgtgtctcaccctgtatctctctgccaggctctgtctctcggtctctgtctcacctgtgccttctccctactgagcacacgcatgggatgggcctggggggaccctgagaaaaggaagggcttt

SEQ ID NO: 24 - DNA, 328 bp, Homo sapiens
ggagagctgtgtcaccatgtgggtcccggttgtcttcctcaccctgtccgtgacgtggattgcaccccctctgcaggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagc

SEQ ID NO: 25 - DNA, 226 bp, Homo sapiens
ggagagctgtgtcaccatgtgggtcccggttgtcttcctcaccctgagcttgtggcctctcgtggcagggcagtctgcggcggtgttctggtgcacccccagtgggtccccacagctgcccactgcatcaggaagtgagtaggggcctggggtctggggagcaggtgtctgtgtcccagaggaataacagctgggcattttccccaggataacctctaaggccagc

SEQ ID NO: 26 - DNA, 220 bp, Homo sapiens
ggagagctgtgtcaccatgtgggtcccggttgtcttcctcaccctgtccgtgacgtggattgggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagc

SEQ ID NO: 27 - DNA, 805 bp, Homo sapiens
ggagagctgtgtcaccatgtgggtcccggttgtcttcctcaccctgtccgtgacgtggattggagctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctcccttctgaacacacgcacgggatgggcctggggggaccctgagaaaagaaaggaccctggggagcgaagtggaggatacaaccttgggcctgcaggccaggctacctacccacttggaaacccacgccaaagccgcatctacagctgagccactctgagg

SEQ ID NO: 28 - DNA, 684 bp, Homo sapiens
ggagagctgtgtcaccatgtgggtcccggttgtcttcctcaccctgtccgtgacgtggattggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagtctgtttcatcctgaagacacaggccaggtatttcaggtcagcctgccgagatcacggatgctgtgaaggtcatggacctgcccacccaggagccagcactggggaccacctgctacgcctcaggctggggcagcattgaaccagaggagttcttgatgccaaagaaacttcagcgtgtggatctccatgttatttgcaatgacgtgtgtgcgcaagttcagcctcagaaggtgatcaagttcatacggtgtgctggacgctggacagcgggcaaaagcacctgctggggtgattttgggggagcgcttgtctgtaatggtgtggttcaaggtatcatgtcatggtgcagtgatccatctatcctagccgaaaggagttgcctgtccaccaaggtggtgcattaccggaagtggatcaaggacaggcatcgt

SEQ ID NO: 29 - DNA, 560 bp, Homo sapiens
ggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagccaggtgatgactccagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattttgggggcccacttgtctgtaatggtgtgcttcaaggtatcacgtcatggggcagtgaaccatctatcctgcccgaaaggccttccctgtacaccaaggtggtgcattaccggaagtggatcaaggacaccatcgtggccaacccctgagcacccatat

SEQ ID NO: 30 - DNA, 541 bp, Homo sapiens
ggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggccca

SEQ ID NO: 31 - DNA, 525 bp, Homo sapiens
tcctgtgtcagcatgtgggtcccggttgtcttcctcaccctgtccgtgacgtggattggcgctgcgcccctcatcctgtctcggattgtctctcgtggcagggcagtcggcggcggtgttctggtgcacccccagtgggtcctcacagctgcccactgcatcaggaagccaggtgatgactccagccacgacctcatgctgctccgcctgtcagagcctgccgagatcacggatgctgtgaaggtcatggacctgcccacccaggagccagcactggggaccacctgctacgcctcaggctggggcagcattgaaccagaggagtgtacgcctgggccagatgtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggggtgcttcaaggt

SEQ ID NO: 32 - DNA, 524 bp, Homo sapiens
ggagagctgtgtcaccatgtgggtcccggttgtcttcctcaccctgtccgtgacgtggattggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggagcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctggggtgattctgggggcccacttgtctgtaatggtgtgcttcaaggtatcacgtcatggggcagtgaaccatgtgccctgcccgaaaggccttcgctgtacaccaaggtggtgcattaccggaagtggatcaaggacaccatcgtggccaacccctgagcacccctat

SEQ ID NO: 33 - DNA, 430 bp, Homo sapiens
ggagagctgtgtcaccatgtgggtcccggttgtcttcctcaccctgtccgtgacgtggattggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctggggtgattctgggggcccacctgtctgtaa

SEQ ID NO: 34 - DNA, 290 bp, Homo sapiens
ggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcgggtgattctgggggcccacttgtctgtaatggtgtgcttcaagg

SEQ ID NO: 35 - DNA, 414 bp, Homo sapiens
cgtgacgtggattggtgagaggggccatggttggggggatgcaggagagggagccagccctgactgtcaagctgaggctctttccccccaaacccagcaccccagctccctgctcccagctgctttactaaaggggaagttcctgggcatctccgtgtttctctttgtggggctcaaaacctccaaggacctctctcaatgccattggttccttggaccgtatcactggtccacctcctgaggccctcaatcctatcacagtctactgacttttcccattcagctgtgagtgcccaaccctatcccagagaccttgatgcttggcctcccaatcttgccctaggatacccagatgccaaccagacacctccttcttcctagccaggctatctggcctgagacaacaaatgggtc

SEQ ID NO: 36 - DNA, 201 bp, Homo sapiens
tggcaggtgcttgtggcctctcgtggcagggcagtctgcggcggtgttctggtgcacccccagtgggtcctcacagctgcccactgcacctgctacgcctcaggctggggcagcattgaaccagaggagttcttgaccccaaagaaacttcagtgtgtggacctccatgttatttccaatgacgtgtgtgcgcaagttcac

SEQ ID NO: 37 - DNA, 475 bp, Homo sapiens
tggcaggtgcttgtggcctctctcgtggcagggcagtctgcggcggtgttctggtgcgcccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtctcacctgtgccttctctcttctgaacacacgcacgggatgggcctggggggaccctgagaaaaggaagggctttgg

SEQ ID NO: 38 - DNA, 444 bp, Homo sapiens
gtccagcccacaacagtgtttttgcctggcccgtagtcttgaccccaaagaaacttcagtgtgtggacctccatgttacttccaatgacgtgtgtgcgcaagttcaccctcagaaggtgaccaagttcatgctgtgtgctggacgctggacagggggcaaaagcacctgctcggagctggaccctgaagtcccttccccaccggccaggactggagcccctacccctctgttggaatccctgcccaccttcttctgggagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaagg

SEQ ID NO: 39 - DNA, 659 bp, Homo sapiens
gtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgtactgaccatgccagccctgccgatggtcctccatggctccctagtgccctggagaggaggtgtctagtcaaagagtactcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaagg

SEQ ID NO: 40 - DNA, 409 bp, Homo sapiens
gtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaagg

SEQ ID NO: 41 - DNA, 378 bp, Homo sapiens
gtccagcccacaacagtgtttttgcctggcccgtagtcttgaccccaaagaaacttcagtgtgtggacctccatgttacttccaatgacgtgtgtgcgcaagttcaccctcagaaggtgaccaagttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggtgggagtgagcaaaca

SEQ ID NO: 42 - DNA, 76 bp, Homo sapiens
tcccagagaccttgatgcttggcctcccaatcttgccctaggatacccagatgccaccagccaccaacctgcaaac

SEQ ID NO: 43 - DNA, 247 bp, Homo sapiens
tcccagagaccttgatgcttggcctcccaatcttgccctaggatacccagatgccaaccagacacctccttcttcctagccaggctatctggcctgagacaacaaatgggtccctcagtctggcaatgggactctgagaactcctcattccctgactcttagccccagactcttcattcagtggcccacattttccttaggaaaaacatgaagcctctccaccagcaccagccaccaacctgcaaac

SEQ ID NO: 44 - DNA, 73 bp, Homo sapiens
gtcccggttgtcttcctcaccctgtccgtgacgtggattgccaggctatctggcctgagacaacaaatgggtc

SEQ ID NO: 45 - DNA, 255 bp, Homo sapiens
ctgaacacacgcacgggatgggcctggggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggctacctacccacttggaaacccacgccaaagccgcatctacagctgagccactctgagg

SEQ ID NO: 46 - DNA, 70 bp, Homo sapiens
gaacacacgcacgggatgggcctggggggaccctgagaaaagccgcatctacagctgagccactctgagg

SEQ ID NO: 47 - DNA, 286 bp, Homo sapiens
tgactccctcaaggcaataggttattcttacagcagagaggaggttgcctggccggagtgaaggatcggggcagggtgcgagagggaagaaaagacccctcctgcagggcctcacctgggccacaggaggacactgcttttcctctgaggagtcaggaactgtggatggtgctggacagaagcaggacagggcctggctctgccgctgccagaggctgccgctggcctccctatgggatcagactgcagggagggagggcagcagggatgtggagggagtgatgat

SEQ ID NO: 48 - DNA, 273 bp, Homo sapiens
tgactccctcaaggcaataggttattcttacagcacaactcatctgttcctgcgttcagcacacggttactaggcacctgctatgcacccagcactgccctagagcctgggacatagcagtgaacagacagagagcggcccctcccttctgtagcccccaagccagtgaggggcacaggcaggaacagggaccacaacacagaaaagctggagggtgtcaggaggtgatcaggctctcagggagggcagcagggatgtggagggagtgatgat

SEQ ID NO: 49 - DNA, 77 bp, Homo sapiens
tgctggacagaagcaggacagggcctggctcagggtgattctgggggtccacttgtctgtaatggtgtgcttcaagg

SEQ ID NO: 50 - protein, 69 aa, Homo sapiens
MWDLVLSIALSVGCTGAVPLIQSRIVGGWECEKHSQPWQVAVYSHGWAHCGGVLVHPQWVLTAAHCLKK

SEQ ID NO: 51 - protein, 54 aa, Homo sapiens
GAVPLIQSRIVGGWECEKHSQPWQVAVYSHGWAHCGGVLVHPQWVLTAAHCLKK

SEQ ID NO: 52 - protein, 49 aa, Homo sapiens
MWDLVLSIALSVGCTGIARSGWVGTTCLSLKTQARGSLSATASHTRSTI

SEQ ID NO: 53 - protein, 34 aa, Homo sapiens
GIARSGWVGTTCLSLKTQARGSLSATASHTRSTI

SEQ ID NO: 54 - protein, 85 aa, Homo sapiens [sequence omitted]

SEQ ID NO: 55 - protein, 70 aa, Homo sapiens
GAVPLIQSRIVGGWECEKHSQPWQVAVYSHGWAHCGGVLVHPQWVLTAAHCLKNLAPQESSVCEPPSPVQ

SEQ ID NO: 56 - protein, 16 aa, Homo sapiens
LAPQESSVCEPPSPVQ

SEQ ID NO: 57 - protein, 149 aa, Homo sapiens
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKESSVCEPPSPVQ

SEQ ID NO: 58 - protein, 134 aa, Homo sapiens
GAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKESSVCEPPSPVQ

SEQ ID NO: 59 - protein, 12 aa, Homo sapiens
ESSVCEPPSPVQ

SEQ ID NO: 60 - protein, 224 aa, Homo sapiens [sequence omitted]

SEQ ID NO: 61 - protein, 209 aa, Homo sapiens
GAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGVSHPYSQHLEGKGE

SEQ ID NO: 62 - protein, 14 aa, Homo sapiens
VSHPYSQHLEGKGE

SEQ ID NO: 63 - protein, 123 aa, Homo sapiens
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPQPLYNMSLLKHQSLRPDEDSSIEPEE

SEQ ID NO: 64 - protein, 108 aa, Homo sapiens
GAVPLIQSRIVGGWECEKHSQPWQVAVYSHGWAHCGGVLVHPQWVLTAAHCLKKNSQVWLGRHNLFEPEDTGQRVPVSHSFPQPLYNMSLLKHQSLRPDEDSSIEPEE

SEQ ID NO: 65 - protein, 5 aa, Homo sapiens
IEPEE

SEQ ID NO: 66 - protein, 165 aa, Homo sapiens
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEW

SEQ ID NO: 67 - protein, 150 aa, Homo sapiens
GAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEW

SEQ ID NO: 68 - protein, 119 aa, Homo sapiens
MWDLVLSIALSVGCTGEIGGIKEGGRVLTLMLKPFSSHPVPQPRPFSPQFSPDNVPLTLPPEAIWQKTTNAASHPESGTGTFRTPPFLTLCPRPVIQWLAFSMGKTRAPPTTTASSLIP

SEQ ID NO: 69 - protein, 104 aa, Homo sapiens
GEIGGIKEGGRVLTLMLKPFSSHPVPQPRPFSPQFSPDNVPLTLPPEAIWQKTTNAASHPESGTGTFRTPPFLTLCPRPVIQWLAFSMGKTRAPPTTTASSLIP

SEQ ID NO: 70 - protein, 59 aa, Homo sapiens
PEAIWQKTTNAASHPESGTGTFRTPPFLTLCPRPVIQWLAFSMGKTRAPPTTTASSLIP

SEQ ID NO: 71 - protein, 17 aa, Homo sapiens
RRTTSTPNHNGQFSDSL

SEQ ID NO: 72 - protein, 21 aa, Homo sapiens
MWDLVLSIALSVGCTDPSFNG

SEQ ID NO: 73 - protein, 6 aa, Homo sapiens
DPSFNG

SEQ ID NO: 74 - protein, 56 aa, Homo sapiens
MWDLVLSIALSVGCTGEIGGIKEGGRVLTLMLKPFSSHPVPQPRPPNHNGQFSDSL

SEQ ID NO: 75 - protein, 41 aa, Homo sapiens
GEIGGIKEGGRVLTLMLKPFSSHPVPQPRPPNHNGQFSDSL

SEQ ID NO: 76 - protein, 11 aa, Homo sapiens
PNHNGQFSDSL

SEQ ID NO: 77 - protein, 83 aa, Homo sapiens [sequence omitted]

SEQ ID NO: 78 - protein, 71 aa, Homo sapiens [sequence omitted]

SEQ ID NO: 79 - protein, 19 aa, Homo sapiens
ALEPPQRGRTHHAPWPYRN

SEQ ID NO: 80 - protein, 298 aa, Homo sapiens
MWDLVLSIALSVGCTGAVPLIQSRIVGGWECEKHSQPWQVAVYSHGWAHSCFLTHCLYFTTTISPRPLCFSHCFFFFPLESPLSSPAPSTFPHFLSPHASTPFLPRNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 81 - protein, 57 aa, Homo sapiens
SCFLTHCLYFTTTISPRPLCFSHCFFFFPLESPLSSPAPSTFPHFLSPHASTPFLPR

SEQ ID NO: 82 - protein, 70 aa, Homo sapiens
SSMCVHVRGCLSLHSTLCRTSLHPHASPPPPATPHSSDAPVASLLFLSHTVSPLASSLLFLLLSLTSRIL

SEQ ID NO: 83 - protein, 69 aa, Homo sapiens
LNVCACEGVPVIAQHSLQDIPPPSCLSSSPCYSTLLRCPRGLPPFSLPHCITPGFLSAVSPSLSDFPHP

SEQ ID NO: 84 - protein, 46 aa, Homo sapiens [sequence omitted]

SEQ ID NO: 85 - protein, 71 aa, Homo sapiens
SDDEDDLGVAPGIVPTWALTQPPSQAPGPQSLPSTPFSTYPQWVILITELTMPALPMAHLSVMGCFKVSHH

SEQ ID NO: 86 - protein, 53 aa, Homo sapiens
PLQSPHSSPSCRNSQVWLGRHNLFEPEDTGQRVPVSHSFPHPLYNMSLLKHQS

SEQ ID NO: 87 - protein, 45 aa, Homo sapiens
HYRALTPAPAAGIARSGWVGTTCLSLKTQARGSLSATASHTRSTI

SEQ ID NO: 88 - protein, 12 aa, Homo sapiens
TTEPSLQPQLQE

SEQ ID NO: 89 - protein, 51 aa, Homo sapiens
MWVPVVFLTLSVTWIGERGHGGGDAGEGAIPDCQAEALCPPTQHPSSQLLY

SEQ ID NO: 90 - protein, 36 aa, Homo sapiens
GERGHGGGDAGEGAIPDCQAEALCPPTQHPSSQLLY

SEQ ID NO: 91 - protein, 89 aa, Homo sapiens
MWVPVVFLTLSVTWIGERGHGWGDAGEGASPDCQAEALSPPTQHPSPDRELGSFLSLPAPLQAHTPSPSHLPQNFLPIAQPAPCSQLLY

SEQ ID NO: 92 - protein, 74 aa, Homo sapiens
GERGHGWGDAGEGASPDCQAEALSPPTQHPSPDRELGSFLSLPAPLQAHTPSPSHLPQNFLPIAQPAPCSQLLY

SEQ ID NO: 93 - protein, 20 aa, Homo sapiens
HLPQNFLPIAQPAPCSQLLY

SEQ ID NO: 94 - protein, 47 aa, homo sapiens
MWVPVVFLTLSVTWIAVSVQPYPRDLDAWPPNLALGYPDANQTPPSS

SEQ ID NO: 95 - protein, 32 aa, Homo sapiens
AVSVQPYPRDLDAWPPNLALGYPDANQTPPSS

SEQ ID NO: 96 - protein, 68 aa, Homo sapiens
MWVPVVFLTLSVTWIGERGHGWGDAGEGASPDCQAEALSPQPYPRDLDAWPPNLALGYPDANQTPPSS

SEQ ID NO: 97 - protein, 53 aa, Homo sapiens
GERGHGWGDAGEGASPDCQAEALSPQPYPRDLDAWPPNLALGYPDANQTPPSS

SEQ ID NO: 98 - protein, 28 aa, Homo sapiens
QPYPRDLDAWPPNLALGYPDANQTPPSS

SEQ ID NO: 99 - protein, 23 aa, Homo sapiens
MWVPVVFPNPIPETLMLGLPILP

SEQ ID NO: 100 - protein, 27 aa, Homo sapiens
MLVPVVFLTLSVTWIGYPDANQTPPSS

SEQ ID NO: 101 - protein, 12 aa, Homo sapiens
GYPDANQTPPSS

SEQ ID NO: 102 - protein, 69 aa, Homo sapiens
MWVPVVFLTLSVTWIGAAPLILSRIVGGWECEKHSQPWQVLVASRGRAVCGGVLVHPQWVLTAAHCIRK

SEQ ID NO: 103 - protein, 54 aa, Homo sapiens
GAAPLILSRIVGGWECEKHSQPWQVLVASRGRAVCGGVLVHPQWVLTAAHCIRK

SEQ ID NO: 104 - protein, 83 aa, Homo sapiens [sequence omitted]

SEQ ID NO: 105 - protein, 68 aa, Homo sapiens
GAAPLILSRIVGGWECEKHSQPWQVLVASRGRAVCGGVLVHPQWVLTAAHCIRNLHIPPSLCPSLVPL

SEQ ID NO: 106 - protein, 14 aa, Homo sapiens
LHIPPSLCPSLVPL

SEQ ID NO: 107 - protein, 75 aa, Homo sapiens
MWVPVVFLTLSVTWIAPPLQALRPSSCLGLWEAGSARSIPNPGRCLWPLVAGQSAAVFWCTPSGSSQLPTASGSE

SEQ ID NO: 108 - protein, 60 aa, Homo sapiens
APPLQALRPSSCLGLWEAGSARSIPNPGRCLWPLVAGQSAAVFWCTPSGSSQLPTASGSE

SEQ ID NO: 109 - protein, 41 aa, Homo sapiens
MWVPVVFLTLSLWPLVAGQSAAVFWCTPSGSPQLPTASGSE

SEQ ID NO: 110 - protein, 30 aa, Homo sapiens
LWPLVAGQSAAVFWCTPSGSPQLPTASGSE

SEQ ID NO: 111 - protein, 39 aa, Homo sapiens
MWVPVVFLTLSVTWIGQSAAVFWCTPSGSSQLPTASGSE

SEQ ID NO: 112 - protein, 24 aa, Homo sapiens
GQSAAVFWCTPSGSSQLPTASGSE

SEQ ID NO: 113 - protein, 69 aa, Homo sapiens; identical to SEQ ID NO: 102

SEQ ID NO: 114 - protein, 54 aa, Homo sapiens; identical to SEQ ID NO: 103

SEQ ID NO: 115 - protein, 100 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSLPRSRML

SEQ ID NO: 116 - protein, 85 aa, Homo sapiens
GAAPLILSRIVGGWECEKHSQPWQVLVASRGRAVCGGVLVHPQWVLTAAHCIRNKSVILLGRHSLFHPEDTGQVFQVSLPRSRML

SEQ ID NO: 117 - protein, 6 aa, Homo sapiens
PRSRML

SEQ ID NO: 118 - protein, 177 aa, Homo sapiens
MWVPVVFLTLSVTWIGAAPLILSRIVGGWECEKHSQPWQVLVASRGRAVCGGVLVHPQWVLTAAHCIRKPGDDSSIEPEEFLTPKKLQCVDLHVISNDVCAQVHPQKVTKFMLCAGRWTGGKSTCSGDSGGPLVCNGVLQGITSWGSEPSILPERPSLYTKVVHYRKWIKDTIVANP

SEQ ID NO: 119 - protein, 162 aa, Homo sapiens
GAAPLILSRIVGGWECEKHSQPWQVLVASRGRAVCGGVLVHPQWVLTAAHCIRKPGDDSSIEPEEFLTPKKLQCVDLHVISNDVCAQVHPQKVTKFMLCAGRWTGGKSTCSGDSGGPLVCNGVLQGITSWGSEPSILPERPSLYTKVVHYRKWIKDTIVANP

SEQ ID NO: 120 - protein, 220 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPSILPERPSLYTKVVHYRKWIKDTIVANP

SEQ ID NO: 121 - protein, 205 aa, Homo sapiens
GAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSIEPEEFLTPKKLQCVDLHVISNDVCAQVHPQKVTKFMLCAGRWTGGKSTCSGDSGGPLVCNGVLQGITSWGSEPSILPERPSLYTKVVHYRKWIKDTIVANP

SEQ ID NO: 122 - protein, 207 aa, Homo sapiens
MWVPVVFLTLSVTWIGAAPLILSRIVSRGRAVGGGVLVHPQWVLTAAHCIRKPGDDSSHDLMLLRLSEPAEITDAVKVMDLPTQEPALGTTCYASGWGSIEPEECTPGPDVLTPKKLQCVDLHVISNDVCAQVHPQKVTKFMLCAGRWTGGKSTCSGDSGGPLVCNGVLQGITSWGSEPSILPERPSLYTKVVHYRKWIKDTIVANP

SEQ ID NO: 123 - protein, 192 aa, Homo sapiens
GAAPLILSRIVSRGRAVGGGVLVHPQWVLTAAHCIRKPGDDSSHDLMLLRLSEPAEITDAVKVMDLPTQEPALGTTCYASGWGSIEPEECTPGPDVLTPKKLQCVDLHVISNDVCAQVHPQKVTKFMLCAGRWTGGKSTCSGDSGGPLVCNGVLQGITSWGSEPSILPERPSLYTKVVHYRKWIKDTIVANP

SEQ ID NO: 124 - protein, 85 aa, Homo sapiens
MWVPVVFLTLSVTWIGAAPLILSRIVGGWECEKHSQPWQVLVASRGRAVCGGVLVHPQWVLTAAHCIRNLDPKETSVCGPPCYFQ

SEQ ID NO: 125 - protein, 70 aa, Homo sapiens
GAAPLILSRIVGGWECEKHSQPWQVLVASRGRAVCGGVLVHPQWVLTAAHCIRNLDPKETSVCGPPCYFQ

SEQ ID NO: 126 - protein, 16 aa, Homo sapiens
LDPKETSVCGPPCYFQ

SEQ ID NO: 127 - protein, 78 aa, Homo sapiens [sequence omitted]

SEQ ID NO: 128 - protein, 63 aa, Homo sapiens
GAAPLILSRIVGGWECEKHSQPWQVLVASRGRAVCGGVLVHPQWVLTAAHCIRNKSVILLGQH

SEQ ID NO: 129 - protein, 144 aa, Homo sapiens
MWVPVVFLTLSVTWIGAAPLILSRIVGGWECEKHSQPWQVLVASRGRAVCGGVLVHPQWVLTAAHCIRNKSVILLGRVILGAHLSVMVCFKVSRHGAVNHVPCPKGLPCTPRCCITGSGSRTPSWPTPEHPYQLPIVVNLEPWK

SEQ ID NO: 130 - protein, 129 aa, Homo sapiens
GAAPLILSRIVGGWECEKHSQPWQVLVASRGRAVCGGVLVHPQWVLTAAHCIRNKSVILLGRVILGAHLSVMVCFKVSRHGAVNHVPCPKGLPCTPRCCITGSGSRTPSWPTPEHPYQLPIVVNLEPWK

SEQ ID NO: 131 - protein, 67 aa, Homo sapiens
VILGAHLSVMVCFKVSRHGAVNHVPCPKGLPCTPRCCITGSGSRTPSWPTPEHPYQLPIVVNLEPWK

SEQ ID NO: 132 - protein, 90 aa, Homo sapiens
MWVPVVFLTLSVTWIGERGHGWGDAGEGASPDCQAEALSPQTQHPSSLLPAALLKGKFLGISVFLFVGLKTSKDLSQCHWFLGPYHWSTS

SEQ ID NO: 133 - protein, 75 aa, Homo sapiens
GERGHGWGDAGEGASPDCQAEALSPQTQHPSSLLPAALLKGKFLGISVFLFVGLKTSKDLSQCHWFLGPYHWSTS

SEQ ID NO: 134 - protein, 44 aa, Homo sapiens
SLLPAALLKGKFLGISVFLFVGLKTSKDLSQCHWFLGPYHWSTS

SEQ ID NO: 135 - protein, 177 aa, Homo sapiens
MWVPVVFLTLSVTWIGAAPLILSRIVGGWECEKHSQPWQVLVASRGRAVCGGVLVHPQWVLTAAHCTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 136 - protein, 69 aa, Homo sapiens; identical to SEQ ID NO: 102

SEQ ID NO: 137 - protein, 61 aa, Homo sapiens [sequence omitted]

SEQ ID NO: 138 - protein, 147 aa, Homo sapiens [sequence omitted]

SEQ ID NO: 139 - protein, 11 aa, Homo sapiens
VQPTTVFLPGP

SEQ ID NO: 140 - protein, 28 aa, Homo sapiens
SSPQQCFCLARSLDPKETSVCGPPCYFQ

SEQ ID NO: 141 - protein, 85 aa, Homo sapiens
PAHNSVFAWPVVLTPKKLQCVDLHVISNDVCAQVHPQKVTKFMLCAGRWTGGKSTCSWVILITELTMPALPMVLHGSLVPWRGGV

SEQ ID NO: 142 - protein, 11 aa, Homo sapiens
VQPTTVFLPGP

SEQ ID NO: 143 - protein, 28 aa, Homo sapiens
SSPQQCFCLARSLDPKETSVCGPPCYFQ

SEQ ID NO: 144 - protein, 100 aa, Homo sapiens
PAHNSVFAWPVVLTPKKLQCVDLHVISNDVCAQVHPQKVTKFMLCAGRWTGGKSTCSVSHPYPSVGIPAHLLLEVGSGDISLFFQSWELLSVICLSRSER

SEQ ID NO: 145 - protein, 11 aa, Homo sapiens
VQPTTVFLPGP

SEQ ID NO: 146 - protein, 28 aa, Homo sapiens
SSPQQCFCLARSLDPKETSVCGPPCYFQ

SEQ ID NO: 147 - protein, 61 aa, Homo sapiens
PAHNSVFAWPVVLTPKKLQCVDLHVTSNDVCAQVHPQKVTKFSTRLLGTCYAPSTALEPGT

SEQ ID NO: 148 - protein, 4 aa, Homo sapiens
SQRP

SEQ ID NO: 149 - protein, 25 aa, Homo sapiens
PRDLDAWPPNLALGYPDATSHQPAN

SEQ ID NO: 150 - protein, 12 aa, Homo sapiens
PETLMLGLPILP

SEQ ID NO: 151 - protein, 4 aa, Homo sapiens
SQRP

SEQ ID NO: 152 - protein, 25 aa, Homo sapiens
PRDLDAWPPNLALGYPDANQTPPSS

SEQ ID NO: 153 - protein, 12 aa, Homo sapiens
PETLMLGLPILP

SEQ ID NO: 154 - protein, 26 aa, Homo sapiens
MWVPVVFLTLSVTWIARLSGLRQQMG

SEQ ID NO: 155 - protein, 11 aa, Homo sapiens
ARLSGLRQQMG

SEQ ID NO: 156 - protein, 80 aa, Homo sapiens [sequence omitted]

SEQ ID NO: 157 - protein, 11 aa, Homo sapiens
EHTHGMGLGGP

SEQ ID NO: 158 - protein, 11 aa, Homo sapiens
EHTHGMGLGGP

SEQ ID NO: 159 - protein, 18 aa, Homo sapiens
NTRTGWAWGDPEKSRIYS

SEQ ID NO: 160 - protein, 21 aa, Homo sapiens
THARDGPGGTLRKAASTAEPL

SEQ ID NO: 161 - protein, 19 aa, Homo sapiens
DSLKAIGYSYSREEVAWPE

SEQ ID NO: 162 - protein, 5 aa, Homo sapiens
TPSRQ

SEQ ID NO: 163 - protein, 23 aa, Homo sapiens
DSLKAIGYSYSTTHLFLRSAHGY

SEQ ID NO: 164 - protein, 5 aa, Homo sapiens
TPSRQ

SEQ ID NO: 165 - protein, 25 aa, Homo sapiens
CWTEAGQGLAQGDSGGPLVCNGVLQ

SEQ ID NO: 166 - protein, 25 aa, Homo sapiens
AGQKQDRAWLRVILGVHLSVMVCFK

SEQ ID NO: 167 - protein, 11 aa, Homo sapiens
LDRSRTGPGSG

SEQ ID NO: 168 - DNA, 24 bp, Homo sapiens
ccagcaccccagctcccagctgct

SEQ ID NO: 169 - DNA, 24 bp, Homo sapiens
ccatacccccagcccctcccactt

SEQ ID NO: 170 - DNA, 24 bp, Homo sapiens
gtgacgtggattgctgtgagtgtc

SEQ ID NO: 171 - DNA, 24 bp, Homo sapiens
aggctctttccccccaaccctatc

SEQ ID NO: 172 - DNA, 24 bp, Homo sapiens
gtgacgtggattggatacccagat

SEQ ID NO: 173 - DNA, 24 bp, Homo sapiens
tccgcctcttattccattctttct

SEQ ID NO: 174 - DNA, 24 bp, Homo sapiens
gcatcaggaatctccatatccccc

SEQ ID NO: 175 - DNA, 24 bp, Homo sapiens
tgacgtggattgcaccccctctgc

SEQ ID NO: 176 - DNA, 24 bp, Homo sapiens
gtcttcctcaccctgagcttgtgg

SEQ ID NO: 177 - DNA, 24 bp, Homo sapiens
cttcctcaccctgagcttgtggcc

SEQ ID NO: 178 - DNA, 24 bp, Homo sapiens
tgacgtggattgggcagtctgcgg

SEQ ID NO: 179 - DNA, 24 bp, Homo sapiens
gagaaaagaaaggaccctggggag

SEQ ID NO: 180 - DNA, 24 bp, Homo sapiens
atttcaggtcagcctgccgagatc

SEQ ID NO: 181 - DNA, 24 bp, Homo sapiens
ctgcatcaggaagccaggtgatga

SEQ ID NO: 182 - DNA, 24 bp, Homo sapiens
gtgatgactccagcattgaaccag

SEQ ID NO: 183 - DNA, 24 bp, Homo sapiens
gtctcggattgtctctcgtggcag

SEQ ID NO: 184 - DNA, 24 bp, Homo sapiens
ctgggccagatgtcttgaccccaa

SEQ ID NO: 185 - DNA, 25 bp, Homo sapiens
tgcatcaggaatcttgaccccaaag

SEQ ID NO: 186 - DNA, 24 bp, Homo sapiens
ttgctgggtcagcattgaaccaga

SEQ ID NO: 187 - DNA, 24 bp, Homo sapiens
atcttgctgggtcgggtgattctg

SEQ ID NO: 188 - DNA, 24 bp, Homo sapiens
cttgctgggtcgggtgattctggg

SEQ ID NO: 189 - DNA, 24 bp, Homo sapiens
ctgcccactgcacctgctacgcct

SEQ ID NO: 190 - DNA, 25 bp, Homo sapiens
ccctctcttctctgtctcacctgtg

SEQ ID NO: 191 - DNA, 24 bp, Homo sapiens
agcacctgctcggagctggaccct

SEQ ID NO: 192 - DNA, 24 bp, Homo sapiens
aagcacctgctcgtgggtcattct

SEQ ID NO: 193 - DNA, 24 bp, Homo sapiens
cacctgctcggtgagtcatcccta

SEQ ID NO: 194 - DNA, 24 bp, Homo sapiens
agaaggtgaccaagttcagcacac

SEQ ID NO: 195 - DNA, 24 bp, Homo sapiens
acccagatgccaccagccaccaac

SEQ ID NO: 196 - DNA, 24 bp, Homo sapiens
ccttaggaaaaacatgaagcctct

SEQ ID NO: 197 - DNA, 24 bp, Homo sapiens
gtgacgtggattgccaggctatct

SEQ ID NO: 198 - DNA, 24 bp, Homo sapiens
aaaattagccaggctacctaccca

SEQ ID NO: 199 - DNA, 24 bp, Homo sapiens
ccctgagaaaagccgcatctacag

SEQ ID NO: 200 - DNA, 25 bp, Homo sapiens
ggttattcttacagcagagaggagg

SEQ ID NO: 201 - DNA, 24 bp, Homo sapiens
gctctcagggagggcagcagggat

SEQ ID NO: 202 - DNA, 24 bp, Homo sapiens
ggcctggctcagggtgattctggg

SEQ ID NO: 203 - DNA, 24 bp, Homo sapiens
tcacttctcaggaatagccaggtc

SEQ ID NO: 204 - DNA, 24 bp, Homo sapiens
gatgttgtgaaggagtcttcagtg

SEQ ID NO: 205 - DNA, 24 bp, Homo sapiens
tggggtgcactggaatagccaggt

SEQ ID NO: 206 - DNA, 24 bp, Homo sapiens
ctggaggggaaagggtgattctgg

SEQ ID NO: 207 - DNA, 24 bp, Homo sapiens
ttgcctaaagaatcttgcgcccca

SEQ ID NO: 208 - DNA, 24 bp, Homo sapiens
gaaccagaggagtgagtcttcagt

SEQ ID NO: 209 - DNA, 24 bp, Homo sapiens
tgaagactccagcatcgaaccaga

SEQ ID NO: 210 - DNA, 24 bp, Homo sapiens
aaccagaggagtggtaaagacact

SEQ ID NO: 211 - DNA, 25 bp, Homo sapiens
ctgactcttccccccgaggctatct

SEQ ID NO: 212 - DNA, 24 bp, Homo sapiens
tggggtgcactgacccgtcattca

SEQ ID NO: 213 - DNA, 24 bp, Homo sapiens
cagcctcgtccccccaaccacaac

SEQ ID NO: 214 - DNA, 24 bp, Homo sapiens
tagtggaaccctgctatctgccga

SEQ ID NO: 215 - DNA, 24 bp, Homo sapiens
ttttctcaggaatagccaggtctg

SEQ ID NO: 216 - DNA, 24 bp, Homo sapiens
gatgggcacactcctgttttctaa

SEQ ID NO: 217 - DNA, 24 bp, Homo sapiens
acatccctccaccctcatgcctct

SEQ ID NO: 218 - DNA, 24 bp, Homo sapiens
agtctctcccctccactccattct

SEQ ID NO: 219 - DNA, 24 bp, Homo sapiens
cctgccgatggcccacttgtctgt

SEQ ID NO: 220 - DNA, 24 bp, Homo sapiens
ccccagctgcaggaatagccaggt